(19) United States
(12) Patent Application Publication
(10) Pub. No.: US 2024/0165271 A1
(43) Pub. Date: May 23, 2024

(54) NUCLEOTIDE EDITING TO REFRAME DMD TRANSCRIPTS BY BASE EDITING AND PRIME EDITING

(71) Applicant: The Board of Regents of The University of Texas System, Austin, TX (US)

(72) Inventors: Eric N. OLSON, Dallas, TX (US); Francesco CHEMELLO, Dallas, TX (US); Rhonda BASSEL-DUBY, Dallas, TX (US)

(73) Assignee: The Board of Regents of The University of Texas System, Austin, TX (US)

(21) Appl. No.: 18/552,262

(22) PCT Filed: Mar. 25, 2022
(86) PCT No.: PCT/US2022/021879

§ 371 Date: Sep. 25, 2023

Related U.S. Application Data
(60) Provisional application No. 63/166,654, filed on Mar. 26, 2021.

(51) Int. Cl. A61K 48/00; A61K 31/7088; A61K 38/46; A61P 21/00; C07K 14/47; C12N 9/22; C12N 15/11; C12N 15/86; C12N 15/90

(52) U.S. Cl. CPC A61K 48/0066; A61K 31/7088; A61K 38/465; A61K 48/0033; A61P 21/00; C07K 14/4716; C12N 9/22; C12N 15/11; C12N 15/86; C12N 15/907; C12N 2310/20; C12N 2750/14143; C12N 2800/80

(57) ABSTRACT

Duchenne muscular dystrophy (DMD) is a fatal muscle disease caused by the lack of dystrophin, which maintains muscle membrane integrity. Provided herein are methods of using adenine base editor (ABE) to modify splice sites of the dystrophin gene, causing skipping or refraining of common DMD exon deletion mutations, restoring dystrophin expression. Also provided herein are methods of using prime editing to reframe the dystrophin open reading frame and restore dystrophin expression.

### PRIORITY CLAIM

This application claims benefit of priority to U.S. Provisional Application Ser. No. 63/166,654, filed Mar. 26, 2021, the entire contents of which are hereby incorporated by reference.

### STATEMENT REGARDING FEDERALLY SPONSORED RESEARCH

This invention was made with government support under Grant No. HD087351 awarded by the National Institutes of Health. The government has certain rights in the invention.

### BACKGROUND

### 1. Field

The present disclosure relates generally to the fields of molecular biology, medicine, and genetics. More particularly, it concerns compositions and uses thereof for genome editing to correct mutations in vivo using a nucleotide editing approach.

### 2. Description of Related Art

Duchenne Muscular Dystrophy (DMD) is a fatal X-linked recessive disorder of progressive neuromuscular weakness and wasting, caused by mutations in the DMD gene that encodes the dystrophin protein (Hoffman et al., 1987). While there are thousands of documented clinical mutations, the majority of DMD-causing mutations occur in a ‘hot spot’ region encompassing exons 45-55 of the DMD gene which encodes the central rod domain of the protein (Flanigan et al., 2009). Mutations in the DMD gene most commonly involve single- or multi-exon deletions that disrupt the open reading frame (ORF) and introduce a premature stop codon that results in production of a non-functional truncated dystrophin protein and causes a severe muscle degeneration phenotype (Muntoni et al., 2003).

The use of myoediting, defined as the CRISPR-Cas9 genome editing in muscle, to permanently correct DMD mutations has been previously demonstrated. Myoediting restores the production of a truncated but functional dystrophin protein in human induced pluripotent stem cell (iPSC)-derived cardiomyocytes, mouse models, and large animal models with DMD mutations (Amoasii et al., 2017; Kyrychenko et al., 2017; Amoasii et al., 2018; Long et al., 2018; Min et al., 2019a; Min et al., 2020; Moretti et al., 2020). These myoediting strategies aimed to ‘reframe’ the correct ORF of the dystrophin transcript by introducing small insertions and deletions (INDELs) via non-homologous end joining (NHEJ) of double-stranded DNA breaks (DSBs) generated by CRISPR-Cas9. Restoration of the ORF was also accomplished via exon skipping by using a single guide RNA (sgRNA) that introduces large INDELs by a ‘single-cut’ at a splice acceptor site (SAS) or splice donor site (SDS), or by using two sgRNAs to introduce a ‘double-cut’ and remove one or more exons (Min et al., 2019b). However, both double- and single-cut myoediting rely on the generation of DSBs in the genome and the NHEJ repair pathway to introduce random INDELs at the cutting site. Methods of correcting DMD mutations without introducing DSBs in the genome are needed.

### SUMMARY

Provided herein are nucleotide gene editing correction strategies to restore dystrophin expression in mice and human cardiomyocytes harboring exon deletion of the DMD gene. In one embodiment, an optimized adenine base editor, ABE (ABEmax; Koblan et al., 2018) fused to SpCas9-NG, packaged into adeno-associated virus 9 (AAV9) using a split-intein system was used to restore dystrophin protein expression in a ΔEx51 DMD mouse model, in which correction strategies have not previously been described. The efficacy of ABE was validated for transcript reframing in the human DMD gene locus by targeting splice sites of exons 50, 51, and 45. The efficacy of prime editing was validated for transcript reframing in human ΔEx51 DMD iPSCs by targeting exon 52. Both of these gene editing tools restored the ORF of the dystrophin transcript and rescued dystrophin protein expression. These findings provide two new gene editing strategies to correct exon deletion mutations in the DMD gene through the most minimal and precise genomic modifications, resulting in restoration of dystrophin protein expression.

In one embodiment, provided herein are guide RNAs (gRNAs) comprising a targeting nucleic acid sequence selected from those disclosed in Table 3. The gRNA may be a single-molecule guide RNA (sgRNA). The gRNA may be for modifying a splice site in the human dystrophin gene.

In one embodiment, provided herein are compositions comprising a gRNA that targets a splice site of one of exons 45, 50, and 51 of human DMD and a base editor. The gRNA may comprise a targeting nucleic acid sequence selected from those disclosed in Table 3. The gRNA may be a single-molecule guide RNA (sgRNA). The base editor may be an adenine base editor (ABE).

The base editor may comprise a CRISPR/Cas nuclease linked to an adenosine deaminase. The CRISPR/Cas nuclease may be catalytically impaired. The CRISPR/Cas nuclease may be a Cas9 nuclease, which may be isolated or derived from Streptococcus pyogenes (e.g., spCas9).

In one embodiment, provided herein are nucleic acids comprising: a sequence encoding a first gRNA that targets a splice site in the human dystrophin gene, a sequence encoding a base editor, a sequence encoding a first promoter, wherein the first promoter drives expression of the sequence encoding the first gRNA, and a sequence encoding a second promoter, wherein the second promoter drives expression of the sequence encoding the base editor.

The gRNA may comprise a targeting nucleic acid sequence selected from those disclosed in Table 3. The gRNA may be a single-molecule guide RNA (sgRNA). The base editor may be an adenine base editor (ABE). The base editor may comprise a CRISPR/Cas nuclease linked to an adenosine deaminase. The CRISPR/Cas nuclease may be catalytically impaired. The CRISPR/Cas nuclease may be a Cas9 nuclease, which may be isolated or derived from Streptococcus pyogenes (e.g., spCas9), Staphylococcus aureus (e.g., SaCas9), Staphylococcus auricularis (e.g., SauCas9), or Staphylococcus lugdunensis (e.g., SlugCas9).

The first promoter and/or the second promoter may be a cell-type specific promoter. The cell-type specific promoter may be a muscle-specific promoter, such as, for example, a CD8 promoter and a CK8e promoter. The first promoter may be a U6 promoter, an H1 promoter, or a 7SK promoter.

The nucleic acid may be a DNA or an RNA. The nucleic acid may comprise a polyadenosine (polyA) sequence, which may be a mini polyA sequence. The nucleic acid may be comprised in a composition, which may be comprised in a cell. The nucleic acid may be comprised in a cell, which may be comprised in a composition.

The nucleic acid may be comprised in a vector. The vector may comprise a sequence encoding an inverted terminal repeat (ITR) of a transposable element, such as, for example, a transposon (e.g., a Tn7 transposon). The vector may comprise a sequence encoding a 5′ ITR of a T7 transposon and a sequence encoding a 3′ ITR of a T7 transposon. The vector may be a non-viral vector, such as, for example, a plasmid. The vector may be a viral vector, such as, for example, an adeno-associated viral (AAV) vector or an adenoviral vector. The AAV vector may be replication-defective or conditionally replication defective. The AAV vector may be a recombinant AAV vector. The AAV vector may comprise a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11), AAV9-rh74-HB-P1, AAV9-AAA-P1-SG, AAVrh10, AAVrh74, AAV9P, MyoAAV1A, MyoAAV2A, MyoAAV3A, MyoAAV4A, MyoAAV4C, or MyoAAV4E, or any combination thereof, wherein the number following AAV indicates the AAV serotype. See, e.g., WO2019193119; WO2022053630; Weinmann et al., 2020, Nature Communications, 11:5432; and Tabebordbar et al., 2021, Cell, 184:1-20, each of which is incorporated by reference herein in its entirety.

The vector may be optimized for expression in mammalian cells, such as, for example, human cells.

The vector may be comprised in a composition, which may further comprise a pharmaceutically acceptable carrier. The vector may be comprised in a cell, such as a human cell, a muscle cell, a satellite cell, or an induced pluripotent stem (iPS) cell. The cell may be comprised in a composition.

In one embodiment, provided herein are methods for correcting a dystrophin defect, the methods comprising contacting a cell with a nucleic acid or vector composition of any one of the present embodiments under conditions suitable for expression of the first gRNA and the adenine base editor, wherein the first gRNA forms a complex with the adenine base editor, wherein the complex modifies a dystrophin splice site thereby restoring correct open reading frame of DMD transcript. A cell produced by such a method is also provided.

In one embodiment, provided herein are guide RNAs (gRNAs) comprising a targeting nucleic acid sequence selected from those of Table 4. The gRNA may be a prime editing (pe) gRNA (pegRNA). The gRNA may be for modifying the human dystrophin gene to restore the correct open reading frame of the human dystrophin gene.

The gRNA may comprise a targeting nucleic acid sequence of 5′-GTAATGAGTTCTTCCAACTG-3′ (SEQ ID NO: 1). The gRNA may further comprise a primer binding site comprising a nucleic acid sequence of 5′-TTGGAAGAACTCA-3′ (SEQ ID NO: 2). The gRNA may further comprise a reverse transcriptase template comprising a nucleic acid sequence of 5′-GAGGCGTCCCCAGGT-3′ (SEQ ID NO: 3).

In one embodiment, provided herein are compositions comprising a gRNA that targets exon 52 of the human dystrophin gene and a prime editor. The gRNA may modify the human dystrophin gene to restore the correct open reading frame of the human dystrophin gene. The gRNA may comprise a targeting nucleic acid sequence selected from those of Table 4. The gRNA may be a prime editing (pe) gRNA (pegRNA). The gRNA may be for modifying the human dystrophin gene to restore the correct open reading frame of the human dystrophin gene.

The prime editor may comprise a CRISPR/Cas nuclease linked to a reverse transcriptase. The CRISPR/Cas nuclease may be catalytically impaired. The CRISPR/Cas nuclease may be a Cas9 nuclease, which may be isolated or derived from Streptococcus pyogenes (e.g., spCas9). The composition may further comprise a second-strand nicking sgRNA.

In one embodiment, provided herein are nucleic acids comprising: a sequence encoding a first gRNA that targets the human dystrophin gene, a sequence encoding a prime editor, a sequence encoding a first promoter, wherein the first promoter drives expression of the sequence encoding the first gRNA, and a sequence encoding a second promoter, wherein the second promoter drives expression of the sequence encoding the prime editor.

The prime editor may comprise a CRISPR/Cas nuclease linked to a reverse transcriptase. The CRISPR/Cas nuclease may be catalytically impaired. The CRISPR/Cas nuclease may be a Cas9 nuclease, which may be isolated or derived from Streptococcus pyogenes (e.g., spCas9). The composition may further comprise a second-strand nicking sgRNA.

The first promoter and/or the second promoter may be a cell-type specific promoter. The cell-type specific promoter may be a muscle-specific promoter, such as, for example, a CD8 promoter and a CK8e promoter. The first promoter may be a U6 promoter, an H1 promoter, or a 7SK promoter.

The nucleic acid may be a DNA or an RNA. The nucleic acid may comprise a polyadenosine (polyA) sequence, which may be a mini polyA sequence. The nucleic acid may be comprised in a composition, which may be comprised in a cell. The nucleic acid may be comprised in a cell, which may be comprised in a composition.

The nucleic acid may be comprised in a vector. The vector may comprise a sequence encoding an inverted terminal repeat (ITR) of a transposable element, such as, for example, a transposon (e.g., a Tn7 transposon). The vector may comprise a sequence encoding a 5′ ITR of a T7 transposon and a sequence encoding a 3′ ITR of a T7 transposon. The vector may be a non-viral vector, such as, for example, a plasmid. The vector may be a viral vector, such as, for example, an adeno-associated viral (AAV) vector or an adenoviral vector. The AAV vector may be replication-defective or conditionally replication defective. The AAV vector may be a recombinant AAV vector. The AAV vector may comprise a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11) or any combination thereof. The vector may be optimized for expression in mammalian cells, such as, for example, human cells.

The vector may be comprised in a composition, which may further comprise a pharmaceutically acceptable carrier. The vector may be comprised in a cell, such as a human cell, a muscle cell, a satellite cell, or an induced pluripotent stem (iPS) cell. The cell may be comprised in a composition.

In one embodiment, provided herein are methods for correcting a dystrophin defect, the methods comprising contacting a cell with a nucleic acid or vector composition of any one of the present embodiments under conditions suitable for expression of the first gRNA and the prime editor, wherein the first gRNA forms a complex with the prime editor, wherein the complex modifies a dystrophin splice site thereby inducing selective skipping of a DMD exon. A cell produced by such a method is also provided.

In one embodiment, provided herein are methods of treating muscular dystrophy in a subject in need thereof, the methods comprising administering to the subject a therapeutically effective amount of a pharmaceutical composition of any one of the present embodiments. Use of a therapeutically effective amount of a pharmaceutical composition of any one of the present embodiments for treating muscular dystrophy in a subject in need thereof is also provided.

The composition may be administered locally. The composition may be administered directly to a muscle tissue. The composition may be administered by an intramuscular infusion or injection. The composition may be administered systemically. The composition may be administered by an intravenous infusion or injection.

Following administration of the composition, the subject may exhibit normal dystrophin-positive myofibers, mosaic dystrophin-positive myofibers containing centralized nuclei, or a combination thereof. Following administration of the composition, the subject may exhibit an emergence or an increase in a level of abundance of normal dystrophin-positive myofibers when compared to an absence or a level of abundance of normal dystrophin-positive myofibers prior to administration of the composition. Following administration of the composition, the subject may exhibit an emergence or an increase in a level of abundance of mosaic dystrophin-positive myofibers containing centralized nuclei when compared to an absence or an level of abundance of mosaic dystrophin-positive myofibers containing centralized nuclei prior to administration of the composition. Following administration of the composition, the subject may exhibit a decreased serum CK level when compared to a serum CK level prior to administration of the composition. Following administration of the composition, the subject may exhibit improved grip strength when compared to a grip strength prior to administration of the composition.

The subject may be a neonate, an infant, a child, a young adult, or an adult. The subject may have muscular dystrophy. The subject may be a genetic carrier for muscular dystrophy. The subject may be male or female. The subject may appear to be asymptomatic and a genetic diagnosis may reveal a mutation in one or both copies of a DMD gene that impairs function of the DMD gene product. The subject may present an early sign or symptom of muscular dystrophy, such as, for example, loss of muscle mass or proximal muscle weakness, such may occur in one or both leg(s) and/or a pelvis, followed by one or more upper body muscle(s). The early sign or symptom of muscular dystrophy may further comprise pseudohypertrophy, low endurance, difficulty standing, difficulty walking, difficulty ascending a staircase or a combination thereof. The subject may present a progressive sign or symptom of muscular dystrophy, such as, for example, muscle tissue wasting, replacement of muscle tissue with fat, or replacement of muscle tissue with fibrotic tissue. The subject may present a later sign or symptom of muscular dystrophy, such as, for example, abnormal bone development, curvature of the spine, loss of movement, and paralysis. The subject may present a neurological sign or symptom of muscular dystrophy, such as, for example, intellectual impairment and paralysis.

The administration of the composition may occur prior to the subject presenting one or more progressive, later or neurological signs or symptoms of muscular dystrophy. The subject may be less than 10 years old, less than 5 years old, or less than 2 years old.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. Strategy for in vivo exon skipping mediated by adenine base editing in the ΔEx51 mouse model. (FIG. 1A) Schematic showing exon skipping and exon reframing strategies to restore the correct ORF of the Dmd transcript. Shape and color of boxes of Dmd exons indicate reading frame. Deletion of exon 51 (ΔEx51) in the Dmd gene generates a premature stop codon in exon 52 (red). Restoration of the correct ORF can be obtained by skipping of exon 50 or 52 (gray), or reframing by a precise insertion of 3n+2 nucleotides (nt) or deletion of 3n−1 nt in exon 50 or 52 (green). (FIG. 1B) Illustration of the mEx50 sgRNA-4 binding position in the region of the splice donor site (SDS) (green) of mouse Dmd exon 50. Sequence shows sgRNA (blue) and PAM (red). Adenines in the editable window of ABEmax-SpCas9-NG are numbered, starting from the PAM. (FIG. 1C) Representative Sanger sequencing chromatogram of the genomic region of the exon 50 SDS in mouse N2a cells, after transfection with ABEmax-SpCas9-NG and mEx50 sgRNA-4. (FIG. 1D) Percentages of DNA editing in mouse N2a cells after transfection with ABEmax-SpCas9-NG and mEx50 sgRNA-4. On-target edit (A14) is colored green. Dots and bars represent different transfection experiments and are mean±SEM (n=3). [SEQ ID NOS: 175-177]

FIGS. 2A-B. In vitro testing of candidate sgRNAs for base editing-mediated exon skipping in the ΔEx51 mouse model. (FIG. 2A) Illustration of the binding positions of the 9 sgRNAs suitable for base editing of SAS or SDS (indicated in green) of mouse exons 50 or 52 using ABEmax-SpCas9-NG. [SEQ ID NOS: 178-185] (FIG. 2B) Percentages of A:T to G:C edits of on-target adenines in the mouse N2a neuroblastoma cell line, after transfection with ABEmax-SpCas9-NG (driven by a CMV promoter) and the different sgRNAs (driven by a U6 promoter). Dots and bars represent results of different transfection experiments and are mean±SEM (n=3).

FIGS. 3A-G. Exon skipping by AAV-mediated base editing in the ΔEx51 mouse model. (FIG. 3A) Schematic of the dual adeno-associated virus 9 (AAV9) system for in vivo delivery of ABEmax-SpCas9-NG and two copies of mEx50 sgRNA-4. (FIG. 3B) Overview for the in vivo intramuscular (IM) injection of the dual AAV9 system in the tibialis anterior (TA) muscle of the left leg of postnatal day 12 (P12) ΔEx51 mice. Right leg was injected with saline as control. (FIG. 3C) Percentages of DNA editing of the adenines from TA injected with the dual AAV9 system. On-target adenine (A14) is colored green. Dots and bars represent biological replicates and are mean±SEM (n=3). (FIG. 3D) Alignment of the top 8 off-target sites in mouse DNA. The target adenine (A14) is colored green. [SEQ ID NOS: 133 and 186-193] (FIG. 3E) Percentages of DNA editing of A14 in the top 8 off-target sites from TA injected with the dual AAV9 system. Dots and bars represent biological replicates and are mean±SEM (n=3). (FIG. 3F) RT-PCR analysis of RNA from the TA of wild-type and ΔEx51 mice injected with the dual AAV9 system or saline as control (Ctrl). (FIG. 3G) Sequence of the RT-PCR product of the lower band confirms splicing of exon 49 to exon 52. [SEQ ID NO: 194]

FIGS. 4A-B. Analysis of potential off-target sites in the mouse genome. (FIG. 4A) Potential off-target sites of SpCas9-NG nuclease and mEx50 sgRNA-4 in mouse genomic DNA identified by CRISPOR. Adenines in the editing window of ABEmax-SpCas9-NG are highlighted in red. Off-target cutting frequency determination (CFD) score is indicated for each site. [SEQ ID NOS: 186-193] (FIG. 4B) Percentages of A:T to G:C editing of the adenines in the top 8 off-target sites in the genomic DNA of TA muscles injected with the dual AAV9 system for the expression of ABEmax-SpCas9-NG and mEx50 sgRNA-4 or with saline as control. Dots and bars represent biological replicates and are mean±SEM.

FIGS. 5A-D. Dystrophin restoration following AAV-mediated base editing in the ΔEx51 mouse model. (FIG. 5A) Western blot analysis of dystrophin protein expression in TA muscles of wild-type and ΔEx51 mice three weeks after IM injection of saline as control (Ctrl) or the dual AAV9 system for the expression of ABEmax-SpCas9-NG and mEx50 sgRNA-4. Vinculin is the loading control. (FIG. 5B) Quantification of dystrophin expression from Western blots after normalization to vinculin. Dots and bars represent biological replicates and are mean±SEM (n=3). (FIG. 5C) Immunohistochemistry of dystrophin in TA muscles three weeks after IM injection of the dual AAV9 system. Indicated is the percentage (mean±SEM) of dystrophin-positive myofibers of TA muscles of ΔEx51 mice receiving IM injection of the dual AAV9 system (n=3). Dystrophin is indicated in green. Scale bar, 100 μm. (FIG. 5D) H&E staining of TA muscles from WT, ΔEx51, and corrected ΔEx51 mice three weeks after IM injection. Scale bar, 100 μm.

FIGS. 6A-B. Intramuscular delivery of the dual AAV9 system for base editing-mediated exon skipping restores dystrophin expression in ΔEx51 mice. (FIG. 6A) Representative images of immunohistochemistry of dystrophin of entire sections of TA muscles of a non-injected WT mouse (Ctrl) and a ΔEx51 mouse, and of the same ΔEx51 mouse injected with saline in the right (R) leg or with the dual AAV9 system for the expression of ABEmax-SpCas9-NG and mEx50 sgRNA-4 in the left (L) leg. The dystrophin-positive myofibers of the muscle injected with saline are due to IM injection leakage from the left leg into the bloodstream. Dystrophin is indicated in green. Scale bar, 500 μm. (FIG. 6B) Percentages of dystrophin-positive myofibers of TA muscles of ΔEx51 mice injected with saline or dual AAV9 system for the expression of ABEmax-SpCas9-NG and mEx50 sgRNA-4. Dots and bars represent biological replicates and are mean±SEM (n=3).

FIGS. 7A-C. Histological improvements of skeletal muscle following intramuscular delivery of the dual AAV9 system for base editing-mediated exon skipping in ΔEx51 mice. (FIG. 7A) Representative images of H&E staining of entire section of TA muscles of the same ΔEx51 mouse injected with saline in the right (R) leg or with the dual AAV9 system for the expression of ABEmax-SpCas9-NG and mEx50 sgRNA-4 in the left (L) leg. Scale bar, 500 μm. (FIG. 7B) Size distribution of muscle fibers of TA muscles of wild-type mice (WT) injected with saline, and ΔEx51 mice injected with saline or the dual AAV9 system for the expression of ABEmax-SpCas9-NG and mEx50 sgRNA-4. Size was calculated as minimal Feret's diameter. Muscle fibers were grouped in size classes of 10 μm and the number of fibers in each class was plotted (n=3) (250 muscle fibers per muscle for a total of 750 muscle fibers per condition). (FIG. 7C) Percentage of muscle fibers with centralized nuclei in TA muscles of WT mice injected with saline, and ΔEx51 mice injected with saline or the dual AAV9 system for the expression of ABEmax-SpCas9-NG and mEx50 sgRNA-4. Dots and bars represent biological replicates and are mean±SEM (n=3).

FIGS. 8A-G. Base editing-mediated exon skipping restores dystrophin expression in human ΔEx51 iPSC-derived cardiomyocytes. (FIG. 8A) Gene editing strategy to restore the in-frame ORF by exon skipping using base editing. (FIG. 8B) The hEx50 sgRNA-1 binding position in the region of the SDS of human DMD exon 50 (green). Sequence shows sgRNA (blue) and PAM (red). (FIG. 8C) Percentages of DNA editing of adenines in the editing window of ABEmax-SpCas9 with hEx50 sgRNA-1 following nucleofection in human ΔEx51 iPSCs. On-target edit (A14) is colored green. Dots and bars represent results of different nucleofections and are mean±SEM (n=3). (FIG. 8D) Representative Sanger sequencing chromatogram of the genomic region of the exon 50 SDS of human iPSCs following nucleofection with ABEmax-SpCas9 and hEx50 sgRNA-1. [SEQ ID NO: 197] (FIG. 8E) RT-PCR analysis of RNA from single clones of healthy control (Ctrl), ΔEx51, and corrected ΔEx51 iPSC-derived cardiomyocytes after base editing. (FIG. 8F) Western blot analysis of dystrophin protein expression of Ctrl, ΔEx51, and corrected ΔEx51 iPSC-derived cardiomyocytes. Vinculin is the loading control. (FIG. 8G) Immunocytochemistry of dystrophin in Ctrl, ΔEx51, and corrected ΔEx51 iPSC-derived cardiomyocytes. Dystrophin is indicated in red. Cardiac troponin I is indicated in green. Nuclei are marked by DAPI (blue). Scale bars, 50 μm.

FIGS. 9A-D. Evaluation of candidate sgRNAs for base editing-mediated exon skipping in human ΔEx51 iPSCs. (FIG. 9A) Illustration of hEx52 sgRNA-2 and -3 binding positions in the region of the SAS of human exon 52 (green). Adenines in the editable window of ABEmax-SpCas9 are numbered, starting from the protospacer adjacent motif (PAM) sequence. The on-target adenine in the SAS (A12 for hEx52 sgRNA-2 and A18 for hEx52 sgRNA-3) is indicated in green. [SEQ ID NOS: 198-199] (FIG. 9B) Percentages of A:T to G:C editing of the adenines in the editing window of hEx50 sgRNA-1, hEx52 sgRNA-2, or hEx52 sgRNA-3 in human 293T cells following transient transfection with ABEmax-SpCas9 and the sgRNAs. On-target edit is colored in green and bystander edits are in blue. Dots and bars represent results of different transfection experiments and are mean±SEM (n=3). (FIG. 9C) Sanger sequencing of the RT-PCR product of RNA from ΔEx51 iPSC-derived cardiomyocytes after genome editing by ABEmax-SpCas9 and hEx50 sgRNA-1 confirms that exon 49 spliced directly to exon 52, skipping exon 50. [SEQ ID NOS: 200-202](FIG. 9D) Immunocytochemistry of dystrophin in healthy control (Ctrl), ΔEx51, and corrected ΔEx51 iPSC-derived single cardiomyocytes. Dystrophin is indicated in red. Cardiac troponin I is indicated in green. Nuclei are marked by DAPI stain in blue. Scale bars, 50 μm.

FIGS. 10A-G. Prime editing-mediated exon reframing restores dystrophin expression in human ΔEx51 iPSC-derived cardiomyocytes. (FIG. 10A) Gene editing strategy to restore the in-frame ORF by exon reframing using prime editing. (FIG. 10B) Illustration of the pegRNA used in the following experiments (red) and the target DNA sequence (blue). PAM is indicated in orange, programmed insertion in green. [SEQ ID NOS: 203-207] (FIG. 10C) RT-PCR analysis of RNA from single clones of healthy control (Ctrl), ΔEx51, and corrected ΔEx51 iPSC-derived cardiomyocytes after prime editing with nick-1 or nick-2. (FIG. 10D) Sanger sequencing chromatograms of the RT-PCR product of RNA from ΔEx51 iPSC-derived cardiomyocytes before and after prime editing. [SEQ ID NOS: 208-209](FIG. 10E) Western blot analysis of dystrophin protein expression of Ctrl, ΔEx51, and corrected ΔEx51 iPSC-derived cardiomyocytes. Vinculin is the loading control. (FIG. 10F) Immunocytochemistry of dystrophin in Ctrl, ΔEx51, and corrected ΔEx51 iPSC-derived cardiomyocytes. Dystrophin is indicated in red. Cardiac troponin I is indicated in green. Nuclei are marked by DAPI stain in blue. Scale bars, 50 μm. (FIG. 10G) Percentage of arrhythmic calcium traces of Ctrl, ΔEx51, and corrected ΔEx51 iPSC-derived cardiomyocytes. Dots and bars represent results of different biological replicates (n=216 cells across three biological replicate experiments) and are mean±SEM (n=3). *P<0.05 and **P<0.001 using unpaired two-tailed Student's t test.

FIGS. 11A-C. Optimization of a pegRNA for prime editing-mediated exon reframing of DMD exon. (FIG. 11A) Potential sgRNAs with a NGG protospacer adjacent motif (PAM) sequence in human exon 52 of the DMD gene. Efficiency score was calculated by CRISPOR. The sgRNA with the highest efficiency score (hEx52 sgRNA-4, highlighted in yellow) was used for the design of the pegRNA for the reframing of exon 52. [SEQ ID NOS: 210-222] (FIGS. 11B and 11C) Percentages of editing efficiency of a +2 nucleotides AC insertion at position +1 with respect to the nicking site generated by hEx52 sgRNA-4 from testing of different nucleotide (nt) lengths of (FIG. 11B) the reverse transcription template (RT) or (FIG. 12C) the primer binding site (PBS) in human 293T cells. Dots and bars represent results of different transfection experiments and are mean±SEM (n=3). *P<0.05 and **P<0.01 using unpaired two-tailed Student's t test.

FIGS. 12A-E. Restoration of dystrophin expression and calcium-cycling in ΔEx51 iPSC-derived cardiomyocytes by prime editing. (FIG. 12A) Percentages of sequences with a +2 nucleotides insertion (green) in human ΔEx51 iPSCs after nucleofection with the prime editing system with nick-1 or nick-2. (FIG. 12B) Western blot analysis of dystrophin protein expression of healthy control (Ctrl), ΔEx51, and mixed populations of prime edited ΔEx51 iPSC-derived cardiomyocytes. Vinculin is the loading control. (FIG. 12C) Quantification of dystrophin expression from Western blots after normalization to vinculin. (FIG. 12D) Representative calcium traces from calcium-cycling analysis of healthy control (Ctrl), ΔEx51, and prime edited ΔEx51 iPSC-derived cardiomyocytes. (FIG. 12E) Representative calcium traces of healthy control arrhythmic iPSC-cardiomyocyte. Compared to arrhythmic DMD iPSC-cardiomyocytes, healthy control arrhythmic iPSC-cardiomyocytes can show single early after depolarizations (EAD) in response to isoproterenol, whereas DMD iPSC-cardiomyocytes show a more complex arrhythmic phenotype after exposure to isoproterenol.

FIGS. 13A-G. Base editing restores dystrophin expression in human ΔEx48-50 iPSC-derived cardiomyocytes. (FIG. 13A) Binding position of candidate sgRNAs for adenine base editing of the splice acceptor or donor sites (SAS or SDS) of human DMD exon 50. Target adenines are indicated in red. [SEQ ID NOS: 223-226] (FIG. 13B) Percentages of DNA editing of adenines in human 293T cells following transient transfection with ABE8e-SpCas9-NG and the indicated sgRNAs. The number of the adenine is indicated counting the nucleotides downstream to the PAM sequence. Target adenine is indicated in red. (FIG. 13C) Percentages of DNA editing of adenines in human ΔEx48-50 DMD iPSCs following transient nucleofection of ABE8e-SpCas9-NG and the indicated sgRNA. The number of the adenine is indicated counting the nucleotides downstream to the PAM sequence. Target adenine is indicated in red. (FIG. 13D) RT-PCR analysis of RNA from healthy control (Ctrl), ΔEx48-50, and corrected ABE edited ΔEx48-50 iPSC-derived cardiomyocytes. (FIG. 13E) Sanger sequencing of the RT-PCR product of RNA from healthy control (Ctrl), ΔEx48-50, and corrected ABE edited ΔEx48-50 iPSC-derived cardiomyocytes. The disruption of the canonical SAS permits the splicing machinery to recognize a cryptic SAS in exon 51 with the consequent deletion of 11 nucleotides and the restoration of the correct open reading frame. [SEQ ID NOS: 227-229] (FIG. 13F) Western blot analysis of dystrophin protein expression of healthy control (Ctrl), ΔEx48-50, and corrected ABE edited ΔEx48-50 iPSC-derived cardiomyocytes. Vinculin is the loading control. (FIG. 13G) Immunocytochemistry of dystrophin in healthy control (Ctrl), ΔEx48-50, and corrected ABE edited ΔEx48-50 iPSC-derived cardiomyocytes. Dystrophin is indicated in red. Cardiac troponin I is indicated in green. Nuclei are marked by DAPI (blue).

FIGS. 14A-G. Base editing restores dystrophin expression in human ΔEx44 iPSC-derived cardiomyocytes. (FIG. 14A) Binding position of candidate sgRNAs for adenine base editing of the splice acceptor or donor sites (SAS or SDS) of human DMD exon 45. Target adenines are indicated in red. [SEQ ID NOS: 230-233] (FIG. 14B) Percentages of DNA editing of adenines in human 293T cells following transient transfection with ABE8e-SpCas9 or ABE8e-SpCas9-NG and the indicated sgRNAs. The number of the adenine is indicated counting the nucleotides downstream to the PAM sequence. Target adenine is indicated in red. (FIG. 14C) Percentages of DNA editing of adenines in human ΔEx44 DMD iPSCs following transient nucleofection of ABE8e-SpCas9 and the indicated sgRNA. The number of the adenine is indicated counting the nucleotides downstream to the PAM sequence. Target adenine is indicated in red. (FIG. 14D) RT-PCR analysis of RNA from healthy control (Ctrl), ΔEx44, and corrected ABE edited ΔEx44 iPSC-derived cardiomyocytes. (FIG. 14E) Representative Sanger sequencing of the RT-PCR product of corrected ABE edited ΔEx44 iPSC-derived cardiomyocytes. The splicing of exon 43 to exon 46 restores the correct open reading frame. [SEQ ID NO: 234] (FIG. 14F) Western blot analysis of dystrophin protein expression of healthy control (Ctrl), ΔEx44, and corrected ABE edited ΔEx44 iPSC-derived cardiomyocytes. Vinculin is the loading control. (FIG. 14G) Immunocytochemistry of dystrophin in healthy control (Ctrl), ΔEx44, and corrected ABE edited ΔEx44 iPSC-derived cardiomyocytes. Dystrophin is indicated in red. Cardiac troponin I is indicated in green. Nuclei are marked by DAPI (blue).

FIGS. 15A-G. SauriCas9 and SlugCas9 base editors restore dystrophin expression in human ΔEx44 iPSC-derived cardiomyocytes. (FIG. 15A) Binding position of candidate sgRNAs for adenine base editing of the splice acceptor site of human DMD exon 45. Target adenine (a) is indicated in red. [SEQ ID NOS: 235-236] (FIG. 15B) ABE8eV106W was fused to SauriCas9 (SauCas9) or SlugCas9 to generate compact base editors. Percentages of DNA editing of target adenines in human 293T cells following transient transfection with the compact base editors and the candidate sgRNAs are indicated in the graph. The number of the adenine is indicated counting the nucleotides downstream to the PAM sequence. hEx45g2 and hEx45g3 showed the higher editing efficiency. (FIG. 15C) Sanger sequences of the splice acceptor site (SAS) of human DMD exon 45 of human ΔEx44 iPSCs after nucleofection with the compact base editors and hEx45g2 and hEx45g3. [SEQ ID NOS: 237-241] (FIG. 15D) Percentages of DNA editing of target adenines in human ΔEx44 iPSCs after nucleofection with the compact base editors and hEx45g2 and hEx45g3. (FIG. 15E) RT-PCR analysis of RNA of human ΔEx44 iPSC-derived cardiomyocytes after base editing. The lower band (368 bp) is the results of the skipping of exon 45. (FIG. 15F) Western blot analysis of dystrophin protein expression of human ΔEx44 iPSC-derived cardiomyocytes after base editing. (FIG. 15G) Immunohistochemistry of dystrophin in human ΔEx44 iPSC-derived cardiomyocytes after base editing. Dystrophin is indicated in red, cardiac troponin is indicated in green, nuclei are marked by DAPI.

FIGS. 16A-G. Generation of ΔEx51 human iPSCs using CRISPR-Cas9-mediated genome editing. (FIG. 16A) Schematic showing CRISPR-Cas9-mediated genomic editing of DMD to generate ΔEx51 hiPSCs using SpCas9 and two sgRNAs flanking exon 51 (black). Upon deletion of exon 51, exon 52 (red) becomes out-of-frame with exon 50. (FIG. 16B) Deletion of the genomic region containing DMD exon 51 (1,400 bp). PCR with primers flanking the deleted region (black arrows) generates a band of 2,151 bp in healthy control (Ctrl) iPSCs, and a band of 751 bp in ΔEx51 iPSCs. (FIG. 16C) Sanger sequencing of the ΔEx51 band confirms the deletion of exon 51 and the formation of a new junction between intron 50 and intron 51. [SEQ ID NO: 242] (FIG. 16D) RT-PCR analysis of mRNA from healthy control (Ctrl) and ΔEx51 iPSC-derived cardiomyocytes. Primers are designed in exons 48 and 54 of human DMD transcript. Band size is 922 base pair (bp) for the Ctrl transcript, and 689 bp for the ΔEx51 transcript, indicating the deletion of exon 51 (233 bp). (FIG. 16E) Sanger sequencing of RT-PCR product from ΔEx51 hiPSC-derived cardiomyocytes confirms the deletion of exon 51 at the RNA level, resulting in the splicing of DMD exon 50 to exon 52. [SEQ ID NO: 243] (FIG. 16F) Western blot analysis of dystrophin protein expression of healthy control (Ctrl) and ΔEx51 hiPSC-derived cardiomyocytes. Vinculin is the loading control. (FIG. 16G) Immunocytochemistry of dystrophin in healthy control (Ctrl) and ΔEx51 hiPSC-derived cardiomyocytes. Dystrophin is indicated in red. Cardiac troponin I is indicated in green. Nuclei are marked by DAPI stain in blue. Scale bars, 50 μm.

### DETAILED DESCRIPTION

Duchenne muscular dystrophy (DMD) is a fatal muscle disease caused by the lack of dystrophin, which maintains muscle membrane integrity. Provided herein are methods of using adenine base editors (ABEs) to modify splice sites of the dystrophin gene, causing reframing of DMD transcript in different exon deletions mutations of exon 51 (ΔEx51), exons 48-50 (ΔEx48-50), or exon 44 (ΔEx44) in cardiomyocytes derived from human iPS cells, restoring dystrophin expression. Prime editing was also capable of reframing the dystrophin open reading frame in ΔEx51 cardiomyocytes. Intramuscular injection of ΔEx51 mice with adeno-associated virus serotype-9 encoding ABE components as a split-intein trans-splicing system allowed gene editing and disease correction in vivo. These findings demonstrate the effectiveness of nucleotide editing technologies for the correction of diverse DMD mutations with minimal and precise modification of the genome.

### I. Duchenne Muscular Dystrophy

Duchenne muscular dystrophy (DMD) is a recessive X-linked form of muscular dystrophy, affecting around 1 in 5000 boys, which results in muscle degeneration and premature death. The disorder is caused by a mutation in the gene dystrophin (see GenBank Accession No. NC_000023.11), located on the human X chromosome, which codes for the protein dystrophin (GenBank Accession No. AAA53189), the sequence of which is reproduced below:

(SEQ ID NO: 4)
MLWWEEVEDCYEREDVQKKTFTKWVNAQFSKFGKQHIENLFSDLQ
DGRRLLDLLEGLTGQKLPKEKGSTRVHALNNVNKALRVLQNNNVD
LVNIGSTDIVDGNHKLTLGLIWNIILHWQVKNVMKNIMAGLQQTN
SEKILLSWVRQSTRNYPQVNVINFTTSWSDGLALNALIHSHRPDL
FDWNSVVCQQSATQRLEHAFNIARYQLGIEKLLDPEDVDTTYPDK
KSILMYITSLFQVLPQQVSIEAIQEVEMLPRPPKVTKEEHFQLHH
QMHYSQQITVSLAQGYERTSSPKPRFKSYAYTQAAYVTTSDPTRS
PFPSQHLEAPEDKSFGSSLMESEVNLDRYQTALEEVLSWLLSAED
TLQAQGEISNDVEVVKDQFHTHEGYMMDLTAHQGRVGNILQLGSK
LIGTGKLSEDEETEVQEQMNLLNSRWECLRVASMEKQSNLHRVLM
DLQNQKLKELNDWLTKTEERTRKMEEEPLGPDLEDLKRQVQQHKV
LQEDLEQEQVRVNSLTHMVVVVDESSGDHATAALEEQLKVLGDRW
ANICRWTEDRWVLLQDILLKWQRLTEEQCLFSAWLSEKEDAVNKI
HTTGFKDQNEMLSSLQKLAVLKADLEKKKQSMGKLYSLKQDLLST
LKNKSVTQKTEAWLDNFARCWDNLVQKLEKSTAQISQAVTTTQPS
LTQTTVMETVTTVITREQILVKHAQEELPPPPPQKKRQITVDSEI
RKRLDVDITELHSWITRSEAVLQSPEFAIFRKEGNFSDLKEKVNA
IEREKAEKFRKLQDASRSAQALVEQMVNEGVNADSIKQASEQLNS
RWIEFCQLLSERLNWLEYQNNIIAFYNQLQQLEQMTTTAENWLKI
QPTTPSEPTAIKSQLKICKDEVNRLSGLQPQIERLKIQSIALKEK
GQGPMFLDADFVAFTNHFKQVFSDVQAREKELQTIFDTLPPMRYQ
ETMSAIRTWVQQSETKLSIPQLSVTDYEIMEQRLGELQALQSSLQ
EQQSGLYYLSTTVKEMSKKAPSEISRKYQSEFEEIEGRWKKLSSQ
LVEHCQKLEEQMNKLRKIQNHIQTLKKWMAEVDVFLKEEWPALGD
SEILKKQLKQCRLLVSDIQTIQPSLNSVNEGGQKIKNEAEPEFAS
RLETELKELNTQWDHMCQQVYARKEALKGGLEKTVSLQKDLSEMH
EWMTQAEEEYLERDFEYKTPDELQKAVEEMKRAKEEAQQKEAKVK
LLTESVNSVIAQAPPVAQEALKKELETLTTNYQWLCTRLNGKCKT
LEEVWACWHELLSYLEKANKWLNEVEFKLKTTENIPGGAEEISEV
LDSLENLMRHSEDNPNQIRILAQTLTDGGVMDELINEELETENSR
WRELHEEAVRRQKLLEQSIQSAQETEKSLHLIQESLTFIDKQLAA
YIADKVDAAQMPQEAQKIQSDLTSHEISLEEMKKHNQGKEAAQRV
LSQIDVAQKKLQDVSMKFRLFQKPANFELRLQESKMILDEVKMHL
PALETKSVEQEVVQSQLNHCVNLYKSLSEVKSEVEMVIKTGRQIV
QKKQTENPKELDERVTALKLHYNELGAKVTERKQQLEKCLKLSRK
MRKEMNVLTEWLAATDMELTKRSAVEGMPSNLDSEVAWGKATQKE
IEKQKVHLKSITEVGEALKTVLGKKETLVEDKLSLLNSNWIAVTS
RAEEWLNLLLEYQKHMETFDQNVDHITKWIIQADTLLDESEKKKP
QQKEDVLKRLKAELNDIRPKVDSTRDQAANLMANRGDHCRKLVEP
QISELNHRFAAISHRIKTGKASIPLKELEQFNSDIQKLLEPLEAE
IQQGVNLKEEDFNKDMNEDNEGTVKELLQRGDNLQQRITDERKRE
EIKIKQQLLQTKHNALKDLRSQRRKKALEISHQWYQYKRQADDLL
KCLDDIEKKLASLPEPRDERKIKEIDRELQKKKEELNAVRRQAEG
LSEDGAAMAVEPTQIQLSKRWREIESKFAQFRRLNFAQIHTVREE
TMMVMTEDMPLEISYVPSTYLTEITHVSQALLEVEQLLNAPDLCA
KDFEDLFKQEESLKNIKDSLQQSSGRIDIIHSKKTAALQSATPVE
RVKLQEALSQLDFQWEKVNKMYKDRQGRFDRSVEKWRRFHYDIKI
FNQWLTEAEQFLRKTQIPENWEHAKYKWYLKELQDGIGQRQTVVR
ILNATGEEIIQQSSKTDASILQEKLGSLNLRWQEVCKQLSDRKKR
LEEQKNILSEFQRDLNEFVLWLEEADNIASIPLEPGKEQQLKEKL
EQVKLLVEELPLRQGILKQLNETGGPVLVSAPISPEEQDKLENKL
KQTNLQWIKVSRALPEKQGEIEAQIKDLGQLEKKLEDLEEQLNHL
LLWLSPIRNQLEIYNQPNQEGPFDVQETEIAVQAKQPDVEEILSK
GQHLYKEKPATQPVKRKLEDLSSEWKAVNRLLQELRAKQPDLAPG
LTTIGASPTQTVTLVTQPVVTKETAISKLEMPSSLMLEVPALADF
NRAWTELTDWLSLLDQVIKSQRVMVGDLEDINEMIIKQKATMQDL
EQRRPQLEELITAAQNLKNKTSNQEARTIITDRIERIQNQWDEVQ
EHLQNRRQQLNEMLKDSTQWLEAKEEAEQVLGQARAKLESWKEGP
YTVDAIQKKITETKQLAKDLRQWQTNVDVANDLALKLLRDYSADD
TRKVHMITENINASWRSIHKRVSEREAALEETHRLLQQFPLDLEK
FLAWLTEAETTANVLQDATRKERLLEDSKGVKELMKQWQDLQGEI
EAHTDVYHNLDENSQKILRSLEGSDDAVLLQRRLDNMNFKWSELR
KKSLNIRSHLEASSDQWKRLHLSLQELLVWLQLKDDELSRQAPIG
GDFPAVQKQNDVHRAFKRELKTKEPVIMSTLETVRIFLTEQPLEG
LEKLYQEPRELPPEERAQNVTRLLRKQAEEVNTEWEKLNLHSADW
QRKIDETLERLQELQEATDELDLKLRQAEVIKGSWQPVGDLLIDS
LQDHLEKVKALRGEIAPLKENVSHVNDLARQLTTLGIQLSPYNLS
TLEDLNTRWKLLQVAVEDRVRQLHEAHRDFGPASQHELSTSVQGP
WERAISPNKVPYYINHETQTTCWDHPKMTELYQSLADLNNVRFSA
YRTAMKLRRLQKALCLDLLSLSAACDALDQHNLKQNDQPMDILQI
INCLTTIYDRLEQEHNNLVNVPLCVDMCLNWLLNVYDTGRIGRIR
VLSFKTGIISLCKAHLEDKYRYLFKQVASSTGFCDQRRLGLLLHD
SIQIPRQLGEVASFGGSNIEPSVRSCFQFANNKPEIEAALFLDWM
RLEPQSMVWLPVLHRVAAAETAKHQAKCNICKECPIIGFRYRSLK
HFNYDICQSCFFSGRVAKGHKMHYPMVEYCTPTTSGEDVRDFAKV
LKNKFRTKRYFAKHPRMGYLPVQTVLEGDNMETPVTLINFWPVDS
APASSPQLSHDDTHSRIEHYASRLAEMENSNGSYLNDSISPNESI
DDEHLLIQHYCQSLNQDSPLSQPRSPAQILISLESEERGELERIL
ADLEEENRNLQAEYDRLKQQHEHKGLSPLPSPPEMMPTSPQSPRD
AELIAEAKLLRQHKGRLEARMQILEDHNKQLESQLHRLRQLLEQP
QAEAKVNGTTVSSPSTSLQRSDSSQPMLLRVVGSQTSDSMGEEDL
LSPPQDTSTGLEEVMEQLNNSFPSSRGRNTPGKPMREDTM

In humans, dystrophin mRNA contains 79 exons. Dystrophin mRNA is known to be alternatively spliced, resulting in various isoforms. Exemplary dystrophin isoforms are listed in Table 1:

TABLE 1
Dystrophin isoforms

| | | Protein | |
|---|---|---|---|
| Sequence | Nucleic Acid | Accession | |
| Name | Accession No. | No. | Description |
| DMD | NC_000023.11 | None | Sequence from Human X Chromosome (at |
| Genomic | (positions | | positions Xp21.2 to p21.1) from Assembly |
| Sequence | 31119219 to | | GRCh38.p7 (GCF_000001405.33) |
| | 33339609) | | |
| Dystrophin | NM_000109.3 | NP_000100.2 | Transcript Variant: transcript Dp427c is |
| Dp427c | | | expressed predominantly in neurons of the |
| isoform | | | cortex and the CA regions of the hippocampus. |
| | | | It uses a unique promoter/exon 1 located about |
| | | | 130 kb upstream of the Dp427m transcript |
| | | | promoter. The transcript includes the common |
| | | | exon 2 of transcript Dp427m and has a similar |
| | | | length of 14 kb. The Dp427c isoform contains a |
| | | | unique N-terminal MED sequence, instead of the |
| | | | MLWWEEVEDCY (SEQ ID NO: 5) sequence |
| | | | of isoform Dp427m. The remainder of isoform |
| | | | Dp427c is identical to isoform Dp427m. |
| Dystrophin | NM_004006.2 | NP_003997.1 | Transcript Variant: transcript Dp427m encodes |
| Dp427m | | | the main dystrophin protein found in muscle. As |
| isoform | | | a result of alternative promoter use, exon 1 |
| | | | encodes a unique N-terminal |
| | | | MLWWEEVEDCY aa sequence. |
| | | | [MLWWEEVEDCY = SEQ ID NO: 5] |
| Dystrophin | NM_004009.3 | NP_004000.1 | Transcript Variant: transcript Dp427p1 initiates |
| Dp427p1 | | | from a unique promoter/exon 1 located in what |
| isoform | | | corresponds to the first intron of transcript |
| | | | Dp427m. The transcript adds the common exon |
| | | | 2 of Dp427m and has a similar length (14 kb). |
| | | | The Dp427p1 isoform replaces the |
| | | | MLWWEEVEDCY -start of Dp427m with a |
| | | | unique N-terminal MSEVSSD aa sequence. |
| | | | [MLWWEEVEDCY = SEQ ID NO: 5; |
| | | | MSEVSSD = SEQ ID NO: 6] |
| Dystrophin | NM_004011.3 | NP_004002.2 | Transcript Variant: transcript Dp260-1 uses |
| Dp260-1 | | | exons 30-79, and originates from a |
| isoform | | | promoter/exon 1 sequence located in intron 29 of |
| | | | the dystrophin gene. As a result, Dp260-1 |
| | | | contains a 95 bp exon 1 encoding a unique N- |
| | | | terminal 16 aa MTEIILLIFFPAYFLN-sequence |
| | | | that replaces amino acids 1-1357 of the full- |
| | | | length dystrophin product (Dp427m isoform). |
| | | | [MTEIILLIFFPAYFLN = SEQ ID NO: 7] |
| Dystrophin | NM_004012.3 | NP_004003.1 | Transcript Variant: transcript Dp260-2 uses |
| Dp260-2 | | | exons 30-79, starting from a promoter/exon 1 |
| isoform | | | sequence located in intron 29 of the dystrophin |
| | | | gene that is alternatively spliced and lacks N- |
| | | | terminal amino acids 1-1357 of the full-length |
| | | | dystrophin (Dp427m isoform). The Dp260-2 |
| | | | transcript encodes a unique N-terminal |
| | | | MSARKLRNLSYKK sequence. |
| | | | [MSARKLRNLSYKK = SEQ ID NO: 8] |
| Dystrophin | NM_004013.2 | NP_004004.1 | Transcript Variant: Dp140 transcripts use exons |
| Dp140 | | | 45-79, starting at a promoter/exon 1 located in |
| isoform | | | intron 44. Dp140 transcripts have a long (1 kb) |
| | | | 5′ UTR since translation is initiated in exon 51 |
| | | | (corresponding to aa 2461 of dystrophin). In |
| | | | addition to the alternative promoter and exon 1, |
| | | | differential splicing of exons 71-74 and 78 |
| | | | produces at least five Dp140 isoforms. Of these, |
| | | | this transcript (Dp140) contains all of the exons. |
| Dystrophin | NM_004014.2 | NP_004005.1 | Transcript Variant: transcript Dp116 uses exons |
| Dp116 | | | 56-79, starting from a promoter/exon 1 within |
| isoform | | | intron 55. As a result, the Dp116 isoform |
| | | | contains a unique N-terminal MLHRKTYHVK |
| | | | aa sequence, instead of aa 1-2739 of dystrophin. |
| | | | Differential splicing produces several Dp116- |
| | | | subtypes. The Dp116 isoform is also known as |
| | | | S-dystrophin or apo-dystrophin-2. |
| | | | [MLHRKTYHVK = SEQ ID NO: 9] |
| Dystrophin | NM_004015.2 | NP_004006.1 | Transcript Variant: Dp71 transcripts use exons |
| Dp71 | | | 63-79 with a novel 80- to 100-nt exon containing |
| isoform | | | an ATG start site for a new coding sequence of |
| | | | 17 nt. The short coding sequence is in-frame |
| | | | with the consecutive dystrophin sequence from |
| | | | exon 63. Differential splicing of exons 71 and 78 |
| | | | produces at least four Dp71 isoforms. Of these, |
| | | | this transcript (Dp71) includes both exons 71 |
| | | | and 78. |
| Dystrophin | NM_004016.2 | NP_004007.1 | Transcript Variant: Dp71 transcripts use exons |
| Dp71b | | | 63-79 with a novel 80- to 100-nt exon containing |
| isoform | | | an ATG start site for a new coding sequence of |
| | | | 17 nt. The short coding sequence is in-frame |
| | | | with the consecutive dystrophin sequence from |
| | | | exon 63. Differential splicing of exons 71 and 78 |
| | | | produces at least four Dp71 isoforms. Of these, |
| | | | this transcript (Dp71b) lacks exon 78 and |
| | | | encodes a protein with a different C-terminus |
| | | | than Dp71 and Dp71a isoforms. |
| Dystrophin | NM_004017.2 | NP_004008.1 | Transcript Variant: Dp71 transcripts use exons |
| Dp71a | | | 63-79 with a novel 80- to 100-nt exon containing |
| isoform | | | an ATG start site for a new coding sequence of |
| | | | 17 nt. The short coding sequence is in-frame |
| | | | with the consecutive dystrophin sequence from |
| | | | exon 63. Differential splicing of exons 71 and 78 |
| | | | produces at least four Dp71 isoforms. Of these, |
| | | | this transcript (Dp71a) lacks exon 71. |
| Dystrophin | NM_004018.2 | NP_004009.1 | Transcript Variant: Dp71 transcripts use exons |
| Dp71ab | | | 63-79 with a novel 80- to 100-nt exon containing |
| isoform | | | an ATG start site for a new coding sequence of |
| | | | 17 nt. The short coding sequence is in-frame |
| | | | with the consecutive dystrophin sequence from |
| | | | exon 63. Differential splicing of exons 71 and 78 |
| | | | produces at least four Dp71 isoforms. Of these, |
| | | | this transcript (Dp71ab) lacks both exons 71 and |
| | | | 78 and encodes a protein with a C-terminus like |
| | | | isoform Dp71b. |
| Dystrophin | NM_004019.2 | NP_004010.1 | Transcript Variant: transcript Dp40 uses exons |
| Dp40 | | | 63-70. The 5′ UTR and encoded first 7 aa are |
| isoform | | | identical to that in transcript Dp71, but the stop |
| | | | codon lies at the splice junction of the |
| | | | exon/intron 70. The 3′ UTR includes nt from |
| | | | intron 70 which includes an alternative |
| | | | polyadenylation site. The Dp40 isoform lacks |
| | | | the normal C-terminal end of full-length |
| | | | dystrophin (aa 3409-3685). |
| Dystrophin | NM_004020.3 | NP_004011.2 | Transcript Variant: Dp140 transcripts use exons |
| Dp140c | | | 45-79, starting at a promoter/exon 1 located in |
| isoform | | | intron 44. Dp140 transcripts have a long (1 kb) |
| | | | 5′ UTR since translation is initiated in exon 51 |
| | | | (corresponding to aa 2461 of dystrophin). In |
| | | | addition to the alternative promoter and exon 1, |
| | | | differential splicing of exons 71-74 and 78 |
| | | | produces at least five Dp140 isoforms. Of these, |
| | | | this transcript (Dp140c) lacks exons 71-74. |
| Dystrophin | NM_004021.2 | NP_004012.1 | Transcript Variant: Dp140 transcripts use exons |
| Dp140b | | | 45-79, starting at a promoter/exon 1 located in |
| isoform | | | intron 44. Dp140 transcripts have a long (1 kb) |
| | | | 5′ UTR since translation is initiated in exon 51 |
| | | | (corresponding to aa 2461 of dystrophin). In |
| | | | addition to the alternative promoter and exon 1, |
| | | | differential splicing of exons 71-74 and 78 |
| | | | produces at least five Dp140 isoforms. Of these, |
| | | | this transcript (Dp140b) lacks exon 78 and |
| | | | encodes a protein with a unique C-terminus. |
| Dystrophin | NM_004022.2 | NP_004013.1 | Transcript Variant: Dp140 transcripts use exons |
| Dp140ab | | | 45-79, starting at a promoter/exon 1 located in |
| isoform | | | intron 44. Dp140 transcripts have a long (1 kb) |
| | | | 5′ UTR since translation is initiated in exon 51 |
| | | | (corresponding to aa 2461 of dystrophin). In |
| | | | addition to the alternative promoter and exon 1, |
| | | | differential splicing of exons 71-74 and 78 |
| | | | produces at least five Dp140 isoforms. Of these, |
| | | | this transcript (Dp140ab) lacks exons 71 and 78 |
| | | | and encodes a protein with a unique C-terminus. |
| Dystrophin | NM_004023.2 | NP_004014.1 | Transcript Variant: Dp140 transcripts use exons |
| Dp140bc | | | 45-79, starting at a promoter/exon 1 located in |
| isoform | | | intron 44. Dp140 transcripts have a long (1 kb) |
| | | | 5′ UTR since translation is initiated in exon 51 |
| | | | (corresponding to aa 2461 of dystrophin). In |
| | | | addition to the alternative promoter and exon 1, |
| | | | differential splicing of exons 71-74 and 78 |
| | | | produces at least five Dp140 isoforms. Of these, |
| | | | this transcript (Dp140bc) lacks exons 71-74 and |
| | | | 78 and encodes a protein with a unique C- |
| | | | terminus. |
| Dystrophin | XM_006724469.3 | XP_006724532.1 | |
| isoform | | | |
| X2 | | | |
| Dystrophin | XM_011545467.1 | XP_011543769.1 | |
| isoform | | | |
| X5 | | | |
| Dystrophin | XM_006724473.2 | XP_006724536.1 | |
| isoform | | | |
| X6 | | | |
| Dystrophin | XM_006724475.2 | XP_006724538.1 | |
| isoform | | | |
| X8 | | | |
| Dystrophin | XM_017029328.1 | XP_016884817.1 | |
| isoform | | | |
| X4 | | | |
| Dystrophin | XM_006724468.2 | XP_006724531.1 | |
| isoform | | | |
| X1 | | | |
| Dystrophin | XM_017029331.1 | XP_016884820.1 | |
| isoform | | | |
| X13 | | | |
| Dystrophin | XM_006724470.3 | XP_006724533.1 | |
| isoform | | | |
| X3 | | | |
| Dystrophin | XM_006724474.3 | XP_006724537.1 | |
| isoform | | | |
| X7 | | | |
| Dystrophin | XM_011545468.2 | XP_011543770.1 | |
| isoform | | | |
| X9 | | | |
| Dystrophin | XM_017029330.1 | XP_016884819.1 | |
| isoform | | | |
| X11 | | | |
| Dystrophin | XM_017029329.1 | XP_016884818.1 | |
| isoform | | | |
| X10 | | | |
| Dystrophin | XM_011545469.1 | XP_011543771.1 | |
| isoform | | | |
| X12 | | | |

The murine dystrophin protein has the following amino acid sequence (Uniprot Accession No. P11531):

(SEQ ID NO: 10)
MWWVDCYRDVKKTTKWNASKGKHDNSDDGKRDGTGKKKGSTRVHA
NNVNKARVKNNVDVNGSTDVDGNHKTGWNHWVKNVMKTMAGTNSK
SWVRSTRNYVNVNTSSWSDGANAHSHRDDWNSVVSHSATRHANAK
CGKDDVATTYDKKSMYTSVVSAVMRTSSKVTRHHHMHYSTVSAGY
TSSSKRKSYATAAYVATSDSTSYSHARDKSDSSMTVNDSYTAVSW
SADTRAGSNDVVKHAHGMMDTSHGVGNVGSVGKGKSDAVMNNSRW
CRVASMKSKHKVMDNKKDDWTKTRTKKMGDDKCVHKVDVRVNSTH
MVVVVDSSGDHATAAKVGDRWANCRWTDRWVDKWHTCSTWSKDAM
KNTSGKDNMMSSHKSTKDKKKTMKSSNDSAKNKSVTKMWMNARWD
NTKKSSASAVTTTSTTTVMTVTMVTTRMVKHAKKRTVDSRKRDVD
THSWTRSAVSSAVYRKGNSDKVNAARKAKRKDASRSAAVMANGVN
ASRASNSRWTCSRVNWYTNTYNMTTTANKTSTTSTAKSKCKDVNR
SAKSKKGGMDADVATNHNHDGVRAKKTDTMRYTMSSRTWSSKSVY
SVTYMRGKASSKNGNYSDTVKMAKKASCKYSGHWKKSSVSCKHMN
KRKNHKTKWMAVDVKWAGDAKKKCRVGDTSNSVNGGKKSAASRTR
NTWDHCRVYTRKAKAGDKTVSKDSMHWMTAYRDYKTDTAVMKRAK
AKTKVKTTVNSVAHASAAKKTTTNYWCTRNGKCKTVWACWHSYKA
NKWNVKKTMNVAGTVSNMHHSNNRATTDGGVMDNTNSRWRHAVRK
KSSAKSHSDKAAYTDKVDAAMAKSDTSHSMKKHNGKDANRVSDVA
KKDVSMKRKANRSKMDVKMHATKSVVSSHCVNYKSSVKSVMVKTG
RVKKTNKDRVTAKHYNGAKVTRKKCKSRKMRKMNVTWAATDTTKR
SAVGMSNDSVAWGKATKKKAHKSVTGSKMVGKKTVDKSNSNWAVT
SRVWNYKHMTDNTKWHADDSKKKKDKRKAMNDMRKVDSTRDAAKM
ANRGDHCRKVVSNRRAASHRKTGKASKNSDKAGVNKDNKDMSDNG
TVNRGDNRTDRKRKKTKHNAKDRSRRKKASHWYYKRADDKCDKKA
SRDRKKDRKKKNAVRRAGSNGAAMAVTSKRWRSNARRNAHTHTMV
VTTDMDVSYVSTYTSHASVDHNTCAKDDKSKNKDNSGRDHKKKTA
ASATSMKVKVAVAMDGKHRMYKRGRDRSVKWRHHYDMKVNWNVKK
TNNWHAKYKWYKDGGRAVVRTNATGSSKTDVNKGSSRWHDCKARR
KRKNVSRDNVWADNATGDKVKARGKNTGGAVVSARDKKKKTNWKV
SRAKGVHKDRDHWSRNYNSAGDKVTVHGKADVRSKGHYKKSTVKR
KDRSWAVNHRRTKDRAGSTTGASASTVTVTSVVTKTVSKMSSVAA
DNRAWTTDWSDRVKSRVMVGDDNMKKATDRRTAANKNKTSNARTT
DRRWDVNRRNMKDSTWAKAVGVRGKDSWKGHTVDAKKTTKAKDRR
SVDVANDAKRDYSADDTRKVHMTNNTSWGNHKRVSAATHRDKSWT
ATTANVDASRKKDSRGVRMKWDGTHTDYHNDNGKRSGSDARRDNM
NKWSKKSNRSHASSDWKRHSVWKDDSRAGGDAVKNDHRAKRKTKV
MSTTVRTGKYRRANVTRRKAVNAWDKNRSADWRKDARAADDKRAV
KGSWVGDDSDHKVKARGAKNVNRVNDAHTTGSYNSTDNTRWRVAV
DRVRHAHRDGASHSTSVGWRASNKVYYNHTTTCWDHKMTYSADNN
VRSAYRTAMKRRKACDSSAACDADHNKNDMDNCTTYDRHNNVNVC
VDMCNWNVYDTGRTGRRVSKTGSCKAHDKYRYKVASSTGCDRRGH
DSRGVASGGSNSVRSCANNKAADWMRSMVWVHRVAAATAKHAKCN
CKCGRYRSKHNYDCSCSGRVAKGHKMHYMVYCTTTSGDVRDAKVK
NKRTKRYAKHRMGYVTVGDNMTVTNWVDSAASSSHDDTHSRHYAS
RAMNSNGSYNDSSNSDDHHYCSNDSSRSASSRGRADNRNAYDRKH
HKGSSMMTSSRDAAAKRHKGRARMDHNKSHRRAAKVNGTTVSSST
SRSDSSMRVVGSTSSMGDSDTSTGVMNNSSSRGRNAGKMRDTM

A. Symptoms

Dystrophin is an important component within muscle tissue that provides structural stability to the dystroglycan complex (DGC) of the cell membrane. While both sexes can carry the mutation, females are rarely affected with the skeletal muscle form of the disease.

Symptoms usually appear in boys between the ages of 2 and 3 and may be visible in early infancy. Even though symptoms do not appear until early infancy, laboratory testing can identify children who carry the active mutation at birth. Progressive proximal muscle weakness of the legs and pelvis associated with loss of muscle mass is observed first. Eventually this weakness spreads to the arms, neck, and other areas. Early signs may include pseudohypertrophy (enlargement of calf and deltoid muscles), low endurance, and difficulties in standing unaided or inability to ascend staircases. As the condition progresses, muscle tissue experiences wasting and is eventually replaced by fat and fibrotic tissue (fibrosis). By age 10, braces may be required to aid in walking but most patients are wheelchair dependent by age 12. Later symptoms may include abnormal bone development that leads to skeletal deformities, including curvature of the spine. Due to progressive deterioration of muscle, loss of movement occurs, eventually leading to paralysis. Intellectual impairment may or may not be present but if present, does not progressively worsen as the child ages. The average life expectancy for males afflicted with DMD is around 25.

The main symptom of DMD, a progressive neuromuscular disorder, is muscle weakness associated with muscle wasting with the voluntary muscles being first affected, especially those of the hips, pelvic area, thighs, shoulders, and calves. Muscle weakness also occurs later, in the arms, neck, and other areas. Calves are often enlarged. Symptoms usually appear before age 6 and may appear in early infancy. Other physical symptoms are:
1. Awkward manner of walking, stepping, or running—(patients tend to walk on their forefeet, because of an increased calf muscle tone. Also, toe walking is a compensatory adaptation to knee extensor weakness.).
2. Frequent falls.
3. Fatigue.
4. Difficulty with motor skills (running, hopping, jumping).
5. Lumbar hyperlordosis, possibly leading to shortening of the hip-flexor muscles. This has an effect on overall posture and a manner of walking, stepping, or running.
6. Muscle contractures of Achilles tendon and hamstrings impair functionality because the muscle fibers shorten and fibrose in connective tissue.
7. Progressive difficulty walking.
8. Muscle fiber deformities.
9. Pseudohypertrophy (enlarging) of tongue and calf muscles. The muscle tissue is eventually replaced by fat and connective tissue, hence the term pseudohypertrophy.
10. Higher risk of neurobehavioral disorders (e.g., ADHD), learning disorders (dyslexia), and non-progressive weaknesses in specific cognitive skills (in particular short-term verbal memory), which are believed to be the result of absent or dysfunctional dystrophin in the brain.
11. Eventual loss of ability to walk (usually by the age of 12).
12. Skeletal deformities (including scoliosis in some cases).
13. Trouble getting up from lying or sitting position.

The condition can often be observed clinically from the moment the patient takes his first steps, and the ability to walk usually completely disintegrates between the time the boy is 9 to 12 years of age. Most men affected with DMD become essentially “paralyzed from the neck down” by the age of 21. Muscle wasting begins in the legs and pelvis, then progresses to the muscles of the shoulders and neck, followed by loss of arm muscles and respiratory muscles. Calf muscle enlargement (pseudohypertrophy) is quite obvious. Cardiomyopathy particularly (dilated cardiomyopathy) is common, but the development of congestive heart failure or arrhythmia (irregular heartbeat) is only occasional.

A positive Gowers' sign reflects the more severe impairment of the lower extremity muscles. The child helps himself to get up with upper extremities: first by rising to stand on his arms and knees, and then “walking” his hands up his legs to stand upright. Affected children usually tire more easily and have less overall strength than their peers. Creatine kinase (CPK-MM) levels in the bloodstream are extremely high. An electromyography (EMG) shows that weakness is caused by destruction of muscle tissue rather than by damage to nerves. Genetic testing can reveal genetic errors in the Xp21 gene. A muscle biopsy (immunohistochemistry or immunoblotting) or genetic test (blood test) confirms the absence of dystrophin, although improvements in genetic testing often make this unnecessary.

DMD patients may suffer from:
1. Abnormal heart muscle (cardiomyopathy).
2. Congestive heart failure or irregular heart rhythm (arrhythmia).
3. Deformities of the chest and back (scoliosis).
4. Enlarged muscles of the calves, buttocks, and shoulders (around age 4 or 5). These muscles are eventually replaced by fat and connective tissue (pseudohypertrophy).
5. Loss of muscle mass (atrophy).
6. Muscle contractures in the heels, legs.
7. Muscle deformities.
8. Respiratory disorders, including pneumonia and swallowing with food or fluid passing into the lungs (in late stages of the disease).

B. Causes

DMD is caused by a mutation of the dystrophin gene at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signaling pathways cause water to enter into the mitochondria, which then burst.

In skeletal muscle dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue.

DMD is inherited in an X-linked recessive pattern. Females will typically be carriers for the disease while males will be affected. Typically, a female carrier will be unaware they carry a mutation until they have an affected son. The son of a carrier mother has a 50% chance of inheriting the defective gene from his mother. The daughter of a carrier mother has a 50% chance of being a carrier and a 50% chance of having two normal copies of the gene. In all cases, an unaffected father will either pass a normal Y to his son or a normal X to his daughter. Female carriers of an X-linked recessive condition, such as DMD, can show symptoms depending on their pattern of X-inactivation.

Exon deletions preceding exon 51 of the human DMD gene, which disrupt the open reading frame (ORF) by juxtaposing out of frame exons, represent the most common type of human DMD mutation. Reframing targeting exon 51 can, in principle, restore the DMD ORF in 13% of DMD patients with exon deletions, targeting exon 45 in 8% of DMD patients, targeting exon 53 in 7% of DMD patients, targeting exon 44 in 6% of DMD patients, and targeting exon 43 or 46 or 50 or 52 in 4% of DMD patients.

Mutations within the dystrophin gene can either be inherited or occur spontaneously during germline transmission. A table of exemplary but non-limiting mutations and corresponding models are set forth below:

TABLE 2
Dystrophin mutations and corresponding mouse models

| Deletion, small insertion and | |
|---|---|
| nonsense mutations | Name of Mouse Model |
| Exon 50 | ΔEx50 |
| Exon 44 | ΔEx44 |
| Exon 52 | ΔEx52 |
| Exon 43 | ΔEx43 |
| Exon 45 | ΔEx45 |
| Exon 51 | ΔEx51 |

Mutations vary in nature and frequency. Large genetic deletions are found in about 60-70% of cases, large duplications are found in about 10% of cases, and point mutants or other small changes account for about 15-30% of cases. Bladen et al. (2015), who examined some 7000 mutations, catalogued a total of 5,682 large mutations (80% of total mutations), of which 4,894 (86%) were deletions (1 exon or larger) and 784 (14%) were duplications (1 exon or larger). There were 1,445 small mutations (smaller than 1 exon, 20% of all mutations), of which 358 (25%) were small deletions and 132 (9%) small insertions, while 199 (14%) affected the splice sites. Point mutations totaled 756 (52% of small mutations) with 726 (50%) nonsense mutations and 30 (2%) missense mutations. Finally, 22 (0.3%) mid-intronic mutations were observed. In addition, mutations were identified within the database that would potentially benefit from novel genetic therapies for DMD including stop codon read-through therapies (10% of total mutations) and exon skipping therapy (80% of deletions and 55% of total mutations).

C. Diagnosis

Genetic counseling is advised for people with a family history of the disorder. DMD can be detected with about 95% accuracy by genetic studies performed during pregnancy.

DNA test. The muscle-specific isoform of the dystrophin gene is composed of 79 exons, and DNA testing and analysis can usually identify the specific type of mutation of the exon or exons that are affected. DNA testing confirms the diagnosis in most cases.

Muscle biopsy. If DNA testing fails to find the mutation, a muscle biopsy test may be performed. A small sample of muscle tissue is extracted (usually with a scalpel instead of a needle) and a dye is applied that reveals the presence of dystrophin. Complete absence of the protein indicates the condition. Over the past several years DNA tests have been developed that detect more of the many mutations that cause the condition, and muscle biopsy is not required as often to confirm the presence of DMD.

Prenatal tests. Prenatal tests can tell whether their unborn child has the most common mutations. There are many mutations responsible for DMD, and some have not been identified, so genetic testing only works when family members with DMD have a mutation that has been identified. Prior to invasive testing, determination of the fetal sex is important; while males are sometimes affected by this X-linked disease, female DMD is extremely rare. This can be achieved by ultrasound scan at 16 weeks or more recently by free fetal DNA testing. Chorion villus sampling (CVS) can be done at 11-14 weeks and has a 1% risk of miscarriage. Amniocentesis can be done after 15 weeks and has a 0.5% risk of miscarriage. Fetal blood sampling can be done at about 18 weeks. Another option in the case of unclear genetic test results is fetal muscle biopsy.

D. Treatment

There is no current cure for DMD, and an ongoing medical need has been recognized by regulatory authorities. Phase 1-2a trials with exon skipping treatment for certain mutations have halted decline and produced small clinical improvements in walking. Treatment is generally aimed at controlling the onset of symptoms to maximize the quality of life, and may include the following:
1. Corticosteroids such as prednisolone and deflazacort increase energy and strength and defer severity of some symptoms.
2. Randomized control trials have shown that beta-2-agonists increase muscle strength but do not modify disease progression. Follow-up time for most RCTs on beta2-agonists is only around 12 months and hence results cannot be extrapolated beyond that time frame.
3. Mild, non-jarring physical activity such as swimming is encouraged. Inactivity (such as bed rest) can worsen the muscle disease.
4. Physical therapy is helpful to maintain muscle strength, flexibility, and function.
5. Orthopedic appliances (such as braces and wheelchairs) may improve mobility and the ability for self-care. Form-fitting removable leg braces that hold the ankle in place during sleep can defer the onset of contractures.
6. Appropriate respiratory support as the disease progresses is important.

Comprehensive multi-disciplinary care standards/guidelines for DMD have been developed by the Centers for Disease Control and Prevention (CDC), and are available on the world wide web at treat-nmd.eu/dmd/care/diagnosis-management-DMD.

DMD generally progresses through five stages. During the presymptomatic stage, patients typically show developmental delay, but no gait disturbance. During the early ambulatory stage, patients typically show the Gowers' sign, waddling gait, and toe walking. During the late ambulatory stage, patients typically exhibit an increasingly labored gait and begin to lose the ability to climb stairs and rise from the floor. During the early non-ambulatory stage, patients are typically able to self-propel for some time, are able to maintain posture, and may develop scoliosis. During the late non-ambulatory stage, upper limb function and postural maintenance is increasingly limited.

In some embodiments, treatment is initiated in the presymptomatic stage of the disease. In some embodiments, treatment is initiated in the early ambulatory stage. In some embodiments, treatment is initiated in the late ambulatory stage. In some embodiments, treatment is initiated during the early non-ambulatory stage. In some embodiments, treatment is initiated during the late non-ambulatory stage.

1. Physical Therapy

Physical therapists are concerned with enabling patients to reach their maximum physical potential. Their aim is to:
1. minimize the development of contractures and deformity by developing a program of stretches and exercises where appropriate,
2. anticipate and minimize other secondary complications of a physical nature by recommending bracing and durable medical equipment, and
3. monitor respiratory function and advise on techniques to assist with breathing exercises and methods of clearing secretions.

2. Respiration Assistance

Modern “volume ventilators/respirators,” which deliver an adjustable volume (amount) of air to the person with each breath, are valuable in the treatment of people with muscular dystrophy related respiratory problems. The ventilator may require an invasive endotracheal or tracheotomy tube through which air is directly delivered, but, for some people non-invasive delivery through a face mask or mouthpiece is sufficient. Positive airway pressure machines, particularly bi-level ones, are sometimes used in this latter way. The respiratory equipment may easily fit on a ventilator tray on the bottom or back of a power wheelchair with an external battery for portability.

Ventilator treatment may start in the mid to late teens when the respiratory muscles can begin to collapse. If the vital capacity has dropped below 40% of normal, a volume ventilator/respirator may be used during sleeping hours, a time when the person is most likely to be under ventilating (“hypoventilating”). Hypoventilation during sleep is determined by a thorough history of sleep disorder with an oximetry study and a capillary blood gas. A cough assist device can help with excess mucus in lungs by hyperinflation of the lungs with positive air pressure, then negative pressure to get the mucus up. If the vital capacity continues to decline to less than 30 percent of normal, a volume ventilator/respirator may also be needed during the day for more assistance. The person gradually will increase the amount of time using the ventilator/respirator during the day as needed.

E. Prognosis

DMD is a progressive disease which eventually affects all voluntary muscles and involves the heart and breathing muscles in later stages. The life expectancy is currently estimated to be around 25, but this varies from patient to patient. Recent advancements in medicine are extending the lives of those afflicted. The Muscular Dystrophy Campaign, which is a leading UK charity focusing on all muscle disease, states that “with high standards of medical care young men with Duchenne muscular dystrophy are often living well into their 30s.”

In rare cases, persons with DMD have been seen to survive into the forties or early fifties, with the use of proper positioning in wheelchairs and beds, ventilator support (via tracheostomy or mouthpiece), airway clearance, and heart medications, if required. Early planning of the required supports for later-life care has shown greater longevity in people living with DMD.

Curiously, in the mdx mouse model of Duchenne muscular dystrophy, the lack of dystrophin is associated with increased calcium levels and skeletal muscle myonecrosis. The intrinsic laryngeal muscles (ILM) are protected and do not undergo myonecrosis. ILM have a calcium regulation system profile suggestive of a better ability to handle calcium changes in comparison to other muscles, and this may provide a mechanistic insight for their unique pathophysiological properties. The ILM may facilitate the development of novel strategies for the prevention and treatment of muscle wasting in a variety of clinical scenarios.

### II. Aspects of the Present Disclosure

The results provided herein demonstrate the effectiveness of two different nucleotide genome editing techniques, base editing and prime editing, for the correction of the most common exon deletion mutations in DMD patients.

The efficacy of in vivo gene editing to correct DMD-causing mutations by the introduction of INDELs within or surrounding out-of-frame exons using CRISPR-Cas9 has previously been demonstrated (Amoasii et al., 2017; Amoasii et al., 2018; Min et al., 2019a; Min et al., 2020; Nelson et al., 2019; Long et al., 2016; Nelson et al., 2016; Bengtsson et al., 2017). One approach to restore the correct ORF in mutated dystrophin transcripts, referred to as double-cut myoediting, used CRISPR-Cas9 and a pair of sgRNAs to introduce two cuts in the DNA to remove intervening target exons for exon skipping. However, double-cut myoediting in its current iterations has limitations in its therapeutic applicability due to its low editing efficiency and its generation of unpredictable genome modifications, such as AAV integration and DNA inversion (Nelson et al., 2019). Another genome editing approach, single-cut myoediting, overcomes some of these limitations by using CRISPR-Cas9 and one sgRNA to introduce one cut in the DNA in the proximity of splice sites to cause exon skipping following small deletions or exon reframing following insertion or deletion of appropriate numbers of nucleotides within out-of-frame exons (Amoasii et al., 2017). However, both double- and single-cut myoediting rely on the generation of DSBs in the genome and the NHEJ repair pathway to introduce random INDELs at the cutting site.

The methods provided herein use nucleotide editing, namely base editing or prime editing, to induce exon skipping or exon reframing, to correct the DMD exon deletion mutations. These two technologies do not introduce DSBs in the genome and offer more precision in the final editing outcome as they do not rely on random INDEL generation for gene editing.

To prove efficacy in vivo, ABEmax-SpCas9-NG was delivered in a mouse model as a split-intein dual AAV system to correct the ΔEx51 mutation in post-mitotic skeletal muscle. SpCas9-NG was used because of its more relaxed NG PAM requirement compared to other Cas nucleases with more stringent PAM requirements (Nishimasu et al., 2018). This increases the number of available sgRNAs that are positioned to edit splice acceptor or splice donor sites. ABEmax was used as base editor as it is associated with fewer off-target consequences compared to CBEs (Grunewald et al., 2019; Jin et al., 2019; Zuo et al., 2019). Intramuscular delivery of the split-intein dual AAV system edits the SDS of exon 50 in muscles of the ΔEx51 mouse model.

### III. Base Editing

The engineered CRISPR technologies of base editing and prime editing have expanded the toolbox of gene editing strategies to potentially correct genetic mutations by enabling precise edits at individual nucleotides (Chemello et al., 2020). In base editing, Cas9 nickase (nCas9) or deactivated Cas9 (dCas9) is fused to a deaminase protein, allowing precise single-base pair conversions without DSBs within a defined editing window in relation to the protospacer adjacent motif (PAM) site of a sgRNA (Rees et al., 2018). There are two major classes of DNA base editors: cytosine base editors (CBEs), which convert a C:G base pair into a T:A base pair, and adenine base editors (ABEs), which convert an A:T base pair into a G:C base pair. Recently, an ABE (ABE7.10) was used to introduce a point mutation and a premature stop codon (p.Q871X) in exon 20 of the mouse Dmd gene and to correct that same point mutation (Kim et al., 2017; Ryu et al., 2018). In addition to correcting point mutations, base editors also can be used to induce exon skipping by mutating target DNA bases of splice motifs (Gapinskie et al., 2018). In this regard, a CBE (hAID P182X) was used at various canonical intronic motifs to modulate splicing of different genes, including the DMD gene in DMD patient-derived iPSCs (Yuan et al., 2018). However, CBEs have been reported to introduce Cas-independent off-target editing at both the genome and transcriptome levels (Grunewald et al., 2019; Jin et al., 2019; Zuo et al., 2019; Lee et al., 2020).

The ‘single-swap’ of a nucleotide base pair in the GT SDS consensus sequence was sufficient to induce exon skipping and restore production of an internally deleted but functional dystrophin protein. Deletion of exon 51 eliminates 78 amino acids from the highly redundant central rod domain of dystrophin. Skipping of exon 50 to enable splicing of exon 49 to exon 52 restores the ORF of dystrophin. Because exon 50 encodes only 36 amino acids in the central rod domain, the corrected form of the dystrophin protein contains 97% of the 3,685 amino acids of the full-length dystrophin protein and is therefore expected to be highly functional. In contrast, “microdystrophins” currently used in DMD gene therapy clinical trials contain approximately 30% of the dystrophin protein and are relatively functionally compromised.

One of the potential concerns reported for base editors is off-target editing. The present off-target analysis did not detect any significant off-target edits in the tested sites. Base editors, such as ABEmax, can edit all available base pairs within a defined activity window. These bystander edits can potentially be disadvantageous in some gene editing applications. For exon skipping, however, bystander edits would occur in the intron or in the to-be-skipped exon and thus not affect the final dystrophin transcript, which makes it an attractive gene editing strategy for correction of DMD mutations.

Adenine base editing as a gene therapy has been previously demonstrated in an adult mouse model of DMD harboring a nonsense point mutation in exon 20. Intramuscular injection of dual trans-splicing viral vectors containing the split ABE and one copy of sgRNA into the TA muscle of these DMD mice resulted in restoration of dystrophin expression in a modest percentage of myofibers (Ryu et al., 2018). Their findings of lower dystrophin expression could be due to differences in the editing efficiency of the sgRNA, the ABE system, the age of the injected mice, or the splicing strategy. Their study utilized a trans-splicing dual AAV strategy, which has been shown to have relatively poor transduction efficiency of the packaged transgene compared to single vector or split-intein AAV systems, which limits its therapeutic potential (Tornabene et al., 2019). This is likely due to the need for trans-splicing AAVs to undergo complex intermolecular concatamerization/recombination and subsequent splicing between the two vectors to reconstitute gene expression (Duan et al. 2001). The present studies used a split-intein dual AAV system which reconstitutes the full-length base editor by protein trans-splicing and has been previously shown to be as efficient as a full-length non-split-intein base editor (Levy et al., 2020). That study also demonstrated correction of a nonsense mutation (p.Q871X) for which the human equivalent mutation (p.Q869X) has been clinically documented in only a few patients. Furthermore, nonsense mutations make up only 10% of the more than 7,000 documented DMD-causing mutations and are evenly distributed across all 79 exons of the largest human gene. On the other hand, exon deletion mutations cluster in a hot-spot region of the DMD gene and account for 68% of all total mutations with deletion of exon 51 being the second most common single exon deletion mutation. Correction strategies for skipping of exon 50 would not only benefit the single exon 51 deletion mutation, but also some multi-exon deletion mutations and could be therapeutically applicable to 4% of all DMD patients (Flanigan et al., 2009; Bladen et al., 2015).

To evaluate the clinical applicability of base editing, the ‘single-swap’ strategy was demonstrated to restore dystrophin expression in human iPSCs with deletion of exon 51, or exons 48-50, or exon 44 in the DMD gene. Base editing was originally envisioned to correct disease-causing point mutations in the genome, however its utility in inducing exon skipping has become increasingly more intriguing due to its more flexible applications as a gene correction strategy (Gapinske et al., 2018). ABE was used as a base editor for exon skipping, however other groups also have demonstrated the use of a cytidine deaminase base editor for exon skipping (Yuan et al., 2018). In the future, the continuous optimization of more efficient base editors will further increase the efficiency of ‘single-swap’ based exon skipping using base editors. It should be noted that for exon skipping, the destruction of a splice site can activate new cryptic splice sites around that exon. This alternative splicing in some cases retains the potential of restoring the correct ORF of the transcript. For example, base editing of the SAS of exon 51 induces the splicing machinery to recognize a new cryptic SAS 11 nucleotides downstream of the canonical SAS, causing reframing of the dystrophin transcript and restoration of dystrophin protein expression.

### IV. Prime Editing

The prime editing system is composed of a prime editing guide RNA (pegRNA) and a nCas9 fused to an engineered reverse transcriptase (Anzalone et al., 2019). The pegRNA consists of (from 5′ to 3′) a sgRNA that anneals to a target site, a scaffold for the nCas9, a reverse transcription template (RT template) containing the desired edit, and a primer binding site (PBS) that binds to the non-target strand. The RT template can be programmed to introduce any type of edit, including all possible base transitions and transversions, and insertions and deletions of nucleotides of any length. The prime editing system is further enhanced by including an additional nicking sgRNA that increases editing efficiency by favoring DNA repair to replace the non-edited strand. Notably, prime editing has not been previously demonstrated as a gene editing correction strategy for DMD.

While base editing at the exon 52 splice acceptor site for exon skipping was relatively inefficient in the initial screen, prime editing generated a desired +2 nucleotide insertion within exon 52 for exon reframing, and serves as an additional nucleotide editing strategy. While INDEL profiles from CRISPR-induced DSBs may have some sequence-dependent predictability in insertion and deletion outcomes (Chakrabarti et al., 2019), the INDEL profiles are nonetheless heterogeneous in their outcome and are site-specific. NHEJ-based INDEL correction thus may produce both non-productive edits and productive edits in restoring the ORF. Prime editing has an advantage of specifying the exact insertion or deletion outcome for exon reframing, thereby ensuring that all of the edits are productive in restoring the correct ORF. Furthermore, in NHEJ-based INDEL correction, a non-productive edit prevents the sgRNA from re-annealing to the site and inducing a productive edit. In prime editing, a non-productive event (i.e. no editing as the edited strand is not successfully incorporated leaving the native sequence intact) leaves the sgRNA target site still amenable to re-annealing and another attempt at inducing the desired edit.

This demonstration that prime editing can correct DMD-causing mutations opens interesting new possibilities. Prime editing can theoretically be used to correct all possible point mutations including base pair transitions and transversions, whereas base editors are limited only to transitions of A:T to G:C or C:G to T:A. In addition, theoretically prime editing is not limited to an editing window as base editing. Also, prime editing can be used to destroy splice sites, however, as shown here, the correction of exon(s) deletion mutations by precise exon reframing instead of exon skipping allows retention of the edited exon, therefore minimizing the number of amino acids that are missing from the final dystrophin protein. As prime editing necessitates the coordination of multiple pegRNA components for editing, such as the spacer sequence, the primer binding site (PBS), and the reverse transcriptase (RT) template, it is likely that editing events at off-target sites are minimal. However, a recent study demonstrated that two opposite strand nicks using the PE3 system can cause undesired editing outcomes in mouse zygote injections (Aida et al., 2020). These undesired editing outcomes were reduced by utilizing a sgRNA that is mutation-specific and can nick only after successful editing and resolution of the pegRNA nick (PE3b system).

Nucleotide editing technologies have the potential to eliminate disease-causing mutations following a single treatment. Use of these two technologies as a gene therapy strategy to induce exon skipping or reframing in an exon deletion DMD model is demonstrated herein. These new editing tools and strategies complement previous genome editing approaches developed for the correction of DMD and represent a step toward clinical correction of DMD and other genetic neuromuscular disorders.

### V. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a base editing enzyme or a reverse transcriptase.

Base editors allow efficient installation of single base substitutions in DNA. For example, adenosine deaminases induce adenosine (A) to inosine (I) edits in single-stranded DNA that in turn result in A-to-G transitions after DNA repair or replication. Adenine base editors (ABEs) are fusions of programmable DNA-binding domains (e.g, catalytically impaired RNA-guided CRISPR/Cas nucleases) linked to an engineered adenosine deaminase. In instances where the programmable DNA-binding domain is a CRISPR/Cas nuclease, targeted adenines lie within an “editing window” in the single-stranded (ss) DNA bubble (R-loop) induced by the CRISPR-Cas RNA-protein complex. The most commonly used ABEs comprise an adenosine deaminase heterodimer consisting of E. coli TadA (wild type) fused to an engineered E. coli TadA variant (e.g. ABEmax) or a single engineered E. coli TadA variant (e.g. ABE8e, ABE8eV106W, or ABE8.20-m) as well as a nickase Cas9 and nuclear localization sequences (NLS). ABEs have been used successfully for installation of A-to-G substitutions in multiple cell types and organisms and could potentially reverse a large number of mutations known to be associated with human disease. Examples of ABEs include those described in U.S. Pat. Publn. US20200308571, PCT Publn. WO2020214842, and PCT Publn. WO2021025750, which are each incorporated herein by reference in their entirety. Reference is made to International Publication No. WO 2018/027078, published Aug. 2, 2018; International Publication No. WO 2019/079347 published Apr. 25, 2019; International Publication No. WO 2019/226593, published Nov. 28, 2019; U.S. Patent Publication No. 2018/0073012, published Mar. 15, 2018, which issued as U.S. Pat. No. 10,113,163, on Oct. 30, 2018; and U.S. Patent Publication No. 2017/0121693, published May 4, 2017, which issued as U.S. Pat. No. 10,167,457 on Jan. 1, 2019.

Prime editing is a versatile and precise genome editing method that directly writes new genetic information into a specified DNA site using a CRISPR system working in association with a polymerase (i.e., in the form of a fusion protein or otherwise provided in trans with the CRISPR system), wherein the prime editing system is programmed with a prime editing (pe) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). As such, prime editors allow for prime editing on a target nucleotide sequence in the presence of a pegRNA (or “extended guide RNA”). The term “prime editor” refers to fusion constructs comprising a Cas9 nickase and a reverse transcriptase. The term “prime editor” may refer to the fusion protein or to the fusion protein complexed with a pegRNA, and/or further complexed with a second-strand nicking sgRNA. In some embodiments, the prime editor may also refer to the complex comprising a fusion protein (reverse transcriptase fused to a Cas9), a pegRNA, and a regular guide RNA capable of directing the second-site nicking step of the non-edited strand as described herein. In other embodiments, the reverse transcriptase component of the “prime editor” may be provided in trans. Further examples of prime editors and their use are provided in PCT Publn. WO2020191249, which is incorporated by reference herein in its entirety.

In some aspects, a Cas nuclease and sgRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. Target sites may be 21, 20, 19, 18, 17, 16, 15, 14, 13, 12, 11, or 10 nucleotides in length. The target site may be selected based on its location immediately 5′ of a protospacer adjacent motif (PAM) sequence, such as typically NGG, NG, NAG, NNNRRT, or NNGG. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. The gRNA may be under the control of a constitutive promoter.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia or S. aureus or S. auricularis or S. lugdunensis). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, a Cas9 polypeptide can be a deactivated (e.g., mutated, dCAs9) Cas9 polypeptide, wherein the deactivated Cas9 does not comprise HNH and/or RuvC nickase activities. The HNH and RuvC motifs have been characterized in S. thermophilus (see, e.g., Sapranauskas et al. Nucleic Acids Res. 39:9275-9282 (2011)) and one of skill would be able to identify and mutate these motifs in Cas9 polypeptides from other organisms. For example, the mutations D10A and H840A completely inactivate the nuclease activity of S. pyogenes Cas9. Notably, a Cas9 polypeptide in which the HNH motif and/or RuvC motif is/are specifically mutated so that the nickase activity is reduced, deactivated, and/or absent, can retain one or more of the other known Cas9 functions including DNA, RNA and PAM recognition and binding activities and thus remain functional with regard to these activities, while non-functional with regard to one or both nickase activities.

In an alternative embodiment, the CRISPR enzyme is a Cas protein, preferably Cas9 (having a nucleotide sequence of Genbank accession no NC_002737.2 and a protein sequence of Genbank accession no NP_269215.1). Again, the Cas9 protein may also be modified to improve activity. For example, the Cas9 protein may comprise the D10A amino acid substitution, this nickase cleaves only the DNA strand that is complementary to and recognized by the crRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas 9 protein may also be the VQR variant. Alternatively, the Cas9 protein may be xCas9 (a Streptococcus pyogenes variant that can recognize a broad range of PAM sequences including NG, GAA and GAT). In other alternatives, the Cas9 variant is SpCas9-NG (with a relaxed preference to the third nucleotide of the PAM motif, such that the variant can recognize sequences where the PAM motif is NGN rather than NGG), SaCas9 (from S. aureus that can recognize NNGRR(T) PAM sequences; see Ran, F. A. et al. In vivo genome editing using Staphylococcus aureus Cas9. Nature 520, 186-191, doi:10.1038/nature14299 (2015)), SaCas9-KKH (a variant from S. aureus that can recognize NNNRRT PAM sequences), SauCas9 (from S. auricularis that can recognize NNGG PAM sequences; Genbank accession no WP_107392933.1), or SlugCas9 (from S. lugdunensis M23590 that can recognize NNGG PAM sequences; Genbank accession no WP_002460848.1).

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Each of the guide sequences of Table 3 may further comprise additional nucleotides to form or encode a crRNA, e.g., using any known sequence appropriate for the Cas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

The guide RNA can be considered to comprise a scaffold sequence necessary for endonuclease binding and a spacer sequence required to bind to the genomic target sequence.

An exemplary scaffold sequence suitable for use with SaCas9 to follow the guide sequence at its 3′ end is: GTTTAAGTACTCTGTGCTGGAAACAGCACAGAATCTACTTAAACAAGGCAAAAT GCCGTGTTTATCTCGTCAACTTGTTGGCGAGA (SEQ ID NO: 500) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 500, or a sequence that differs from SEQ ID NO: 500 by no more than 1, 2, 3, 4, 5, 10, 15, 20, or 25 nucleotides.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity, nucleic acid binding activity, base editing activity, or reverse transcription activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

As an RNA guided protein, Cas9 requires a short RNA to direct the recognition of DNA targets. Though Cas9 preferentially interrogates DNA sequences containing a PAM sequence (e.g., NGG or NG or NNNRRT or NNGG) it can bind here without a protospacer target. However, the Cas9-gRNA complex requires a close match to the gRNA to create a double strand break. CRISPR sequences in bacteria are expressed in multiple RNAs and then processed to create guide strands for RNA. Because eukaryotic systems lack some of the proteins required to process CRISPR RNAs, the synthetic construct gRNA was created to combine the essential pieces of RNA for Cas9 targeting into a single RNA expressed with the RNA polymerase type III promoter U6. Other promoters under the control of RNA Pol III include those for ribosomal 5S rRNA, tRNA and few other small RNAs, RNase P and RNase MRP RNA, 7SL RNA (the RNA component of the signal recognition particles), Vault RNAs, Y RNA, SINEs (short interspersed repetitive elements), 7SK RNA, two microRNAs, several small nucleolar RNAs and several few regulatory antisense RNAs. Synthetic gRNAs are slightly over 100 bp at the minimum length and contain a portion which is targets the 20 or 21 protospacer nucleotides immediately preceding the PAM sequence. The length of the sgRNA can also be shortened at the 5′ with respect to its canonical length to meet specific criteria, e.g. the removal of a stretch of thymines that can inhibit the polymerase type III transcription activity. gRNAs do not contain the PAM sequence.

In some embodiments, the gRNA targets a site within a wildtype dystrophin gene. In some embodiments, the gRNA targets a site within a mutant dystrophin gene. In some embodiments, the gRNA targets a dystrophin intron. In some embodiments, the gRNA targets a dystrophin exon. In some embodiments, the gRNA targets a site in a dystrophin exon that is expressed and is present in one or more dystrophin isoform. In embodiments, the gRNA targets a dystrophin splice site. In some embodiments, the gRNA targets a splice donor site on the dystrophin gene. In embodiments, the gRNA targets a splice acceptor site on the dystrophin gene.

In some embodiments, gRNAs of the disclosure comprise a sequence that is complementary to a target sequence within a coding sequence or a non-coding sequence corresponding to the DMD gene, and, therefore, hybridize to the target sequence. The following tables (Tables 3 and 4) provide exemplary gRNA targeting sequences for use in connection with the compositions and methods disclosed herein.

TABLE 3
Target sequences of exemplary gRNAs targeting splice sites of human DMD
exons 43, 44, 45, 46, 50, 51, 52, or 53 in combination with adenine base editors

| | | SEQ ID | | Examples of Cas9 |
|---|---|---|---|---|
| Name | Sequence (5′->3′) | NO: | Target | (fused with ABE) |
| hEx50-sgRNA-1 | ATACTTACAGGC | 11 | hEx50-SDS | SpCas9; SpCas9- |
| | TCCAATAG | | | NG |
| hEx52-sgRNA-2 | GTTCTTACAGGC | 12 | hEx52-SAS | SpCas9; SpCas9- |
| | AACAATGC | | | NG |
| hEx52-sgRNA-3 | ACAGGCAACAAT | 13 | hEx52-SAS | SpCas9; SpCas9- |
| | GCAGGATT | | | NG |
| hEx51-1565256 | AAATATTTTAGC | 14 | hEx51-SAS | SpCas9-NG |
| | TCCTACTC | | | |
| hEx51-1565260 | ATTTTAGCTCCT | 15 | hEx51-SAS | SpCas9-NG |
| | ACTCAGAC | | | |
| hEx51-1565260sh | TTTAGCTCCTAC | 16 | hEx51-SAS | SpCas9-NG |
| | TCAGAC | | | |
| hEx51-1565475 | TTCTCATACCTTC | 17 | hEx51-SDS | SpCas9-NG |
| | TGCTTGA | | | |
| hEx51-1565478sh | TTTCTCATACCTT | 18 | hEx51-SDS | SpCas9-NG |
| | CTGCT | | | |
| hEx45-1370932 | TGGTATCTTACA | 19 | hEx45-SAS | SpCas9; SpCas9- |
| | GGAACTCC | | | NG |
| hEx45-1370933 | GGTATCTTACAG | 20 | hEx45-SAS | SpCas9-NG |
| | GAACTCCA | | | |
| hEx45-1370936 | ATCTTACAGGAA | 21 | hEx45-SAS | SpCas9; SpCas9- |
| | CTCCAGGA | | | NG |
| hEx45-1370937 | TCTTACAGGAAC | 22 | hEx45-SAS | SpCas9-NG |
| | TCCAGGAT | | | |
| hEx45-1370942 | CAGGAACTCCAG | 23 | hEx45-SAS | SpCas9; SpCas9- |
| | GATGGCAT | | | NG |
| hEx45-1370943 | AGGAACTCCAGG | 24 | hEx45-SAS | SpCas9; SpCas9- |
| | ATGGCATT | | | NG |
| hEx45-1371089 | ACCTCTTTTTTCT | 25 | hEx45-SDS | SpCas9-NG |
| | GTCTGAC | | | |
| hEx45-1371093 | CCCTACCTCTTTT | 26 | hEx45-SDS | SpCas9-NG |
| | TTCTGTC | | | |
| hEx45-1371097 | GTCGCCCTACCT | 27 | hEx45-SDS | SpCas9-NG |
| | CTTTTTTC | | | |
| hEx43-1051746 | TTTATATTACAG | 28 | hEx43-SAS | SpCas9-NG |
| | AATATAAA | | | |
| hEx43-1051750 | TATTACAGAATA | 29 | hEx43-SAS | SpCas9-NG |
| | TAAAAGAT | | | |
| hEx43-1051904 | TACCTACCCTTG | 30 | hEx43-SDS | SpCas9-NG |
| | TCGGTCCT | | | |
| hEx43-1051910 | ATGTGTTACCTA | 31 | hEx43-SDS | SpCas9-NG |
| | CCCTTGTC | | | |
| hEx43-1051911 | TATGTGTTACCT | 32 | hEx43-SDS | SpCas9-NG |
| | ACCCTTGT | | | |
| hEx43-1051914 | ATATATGTGTTA | 33 | hEx43-SDS | SpCas9-NG |
| | CCTACCCT | | | |
| hEx44-1122382 | GCTTTTACCTGC | 34 | hEx44-SAS | SpCas9-NG |
| | AGGCGATT | | | |
| hEx44-1122386 | TTACCTGCAGGC | 35 | hEx44-SAS | SpCas9-NG |
| | GATTTGAC | | | |
| hEx44-1122391 | TGCAGGCGATTT | 36 | hEx44-SAS | SpCas9-NG |
| | GACAGATC | | | |
| hEx44-1122394 | AGGCGATTTGAC | 37 | hEx44-SAS | SpCas9-NG |
| | AGATCTGT | | | |
| hEx44-1122516 | ACTTACCTTAAG | 38 | hEx44-SDS | SpCas9-NG |
| | ATACCATT | | | |
| hEx44-1122526 | CAAATCAAAGAC | 39 | hEx44-SDS | SpCas9-NG |
| | TTACCTTA | | | |
| hEx46-1407215 | TATTCTTCTTTCT | 40 | hEx46-SAS | SpCas9-NG |
| | CCAGGCT | | | |
| hEx46-1407218 | TCTTCTTTCTCCA | 41 | hEx46-SAS | SpCas9-NG |
| | GGCTAGA | | | |
| hEx46-1407226 | CTCCAGGCTAGA | 42 | hEx46-SAS | SpCas9-NG |
| | AGAACAAA | | | |
| hEx46-1407353 | AAATTACCTTGA | 43 | hEx46-SDS | SpCas9-NG |
| | CTTGCTCA | | | |
| hEx46-1407359 | AAAATAAAATTA | 44 | hEx46-SDS | SpCas9-NG |
| | CCTTGACT | | | |
| hEx46-1407364 | TTGAGAAAATAA | 45 | hEx46-SDS | SpCas9-NG |
| | AATTACCT | | | |
| hEx50-1519360 | TGCTTTTCTGTTA | 46 | hEx50-SAS | SpCas9-NG |
| | AAGAGGA | | | |
| hEx50-1519364 | TTTCTGTTAAAG | 47 | hEx50-SAS | SpCas9-NG |
| | AGGAAGTT | | | |
| hEx50-1519367 | CTGTTAAAGAGG | 48 | hEx50-SAS | SpCas9-NG |
| | AAGTTAGA | | | |
| hEx50-1519372 | AAAGAGGAAGTT | 49 | hEx50-SAS | SpCas9-NG |
| | AGAAGATC | | | |
| hEx50-1519374 | AGAGGAAGTTAG | 50 | hEx50-SAS | SpCas9-NG |
| | AAGATCTG | | | |
| hEx50-1519454 | TACAGGCTCCAA | 51 | hEx50-SDS | SpCas9-NG |
| | TAGTGGTC | | | |
| hEx50-1519458 | TACTTACAGGCT | 52 | hEx50-SDS | SpCas9-NG |
| | CCAATAGT | | | |
| hEx50-1519461 | GTATACTTACAG | 53 | hEx50-SDS | SpCas9-NG |
| | GCTCCAAT | | | |
| hEx52-1609698 | TTTGTTCTTACA | 54 | hEx52-SAS | SpCas9-NG |
| | GGCAACAA | | | |
| hEx52-1609702 | TTCTTACAGGCA | 55 | hEx52-SAS | SpCas9-NG |
| | ACAATGCA | | | |
| hEx52-1609708 | CAGGCAACAATG | 56 | hEx52-SAS | SpCas9-NG |
| | CAGGATTT | | | |
| hEx52-1609799 | TTACTTCGATCC | 57 | hEx52-SDS | SpCas9-NG |
| | GTAATGAT | | | |
| hEx52-1609803 | AAACTTACTTCG | 58 | hEx52-SDS | SpCas9-NG |
| | ATCCGTAA | | | |
| hEx52-1609808 | TTAAAAAACTTA | 59 | hEx52-SDS | SpCas9-NG |
| | CTTCGATC | | | |
| hEx53-1659865 | TATTCTAGTTGA | 60 | hEx53-SAS | SpCas9-NG |
| | AAGAATTC | | | |
| hEx53-1659871 | AGTTGAAAGAAT | 61 | hEx53-SAS | SpCas9-NG |
| | TCAGAATC | | | |
| hEx53-1660060 | GATACTAACCTT | 62 | hEx53-SDS | SpCas9-NG |
| | GGTTTCTG | | | |
| hEx53-1660062 | TTGATACTAACC | 63 | hEx53-SDS | SpCas9-NG |
| | TTGGTTTC | | | |
| hEx51-SaK- | AGCTCCTACTCA | 64 | hEx51-SAS | SaCas9-KKH; |
| 1565266 | GACTGTTAC | | | SauriCas9; |
| | | | | SlugCas9 |
| hEx51-Sau- | AGCTCCTACTCA | 65 | hEx51-SAS | SauriCas9; |
| 15652671 | GACTGTTACT | | | SlugCas9 |
| hEx51-SaK- | TTTCTCATACCTT | 66 | hEx51-SDS | SaCas9-KKH |
| 1565477sh | CTGCTT | | | |
| hEx45-Sa- | TTTGGTATCTTA | 67 | hEx45-SAS | SaCas9; SaCas9- |
| 1370931 | CAGGAACTC | | | KKH; SauriCas9; |
| | | | | SlugCas9 |
| hEx45-Sau- | GTATCTTACAGG | 68 | hEx45-SAS | SauriCas9; |
| 1370935 | AACTCCAGG | | | SlugCas9 |
| hEx45-Sa- | TACAGGAACTCC | 69 | hEx45-SAS | SaCas9; SauriCas9; |
| 1370941 | AGGATGGCA | | | SlugCas9 |
| hEx45-Sau- | ACAGGAACTCCA | 70 | hEx45-SAS | SauriCas9; |
| 1370942 | GGATGGCAT | | | SlugCas9 |
| hEx53-Sa- | TTTATTCTAGTTG | 71 | hEx53-SAS | SaCas9; SaCas9- |
| 1659864 | AAAGAATT | | | KKH; SauriCas9; |
| | | | | SlugCas9 |
| hEx53-Sa- | AGTTGAAAGAAT | 72 | hEx53-SAS | SaCas9; SauriCas9; |
| 1659872 | TCAGAATCA | | | SlugCas9 |
| hEx53-Sa- | AGTTGAAAGAAT | 73 | hEx53-SAS | SaCas9; SaCas9- |
| 16598731 | TCAGAATCAG | | | KKH; SauriCas9; |
| | | | | SlugCas9 |
| hEx53-SaK- | TTTGATACTAAC | 74 | hEx53-SDS | SaCas9-KKH |
| 1660062 | CTTGGTTTC | | | |
| hEx44-Sa- | GCAGGCGATTTG | 75 | hEx44-SAS | SaCas9 |
| 1122393 | ACAGATCTG | | | |
| hEx44-SaK- | TTTACCTGCAGG | 76 | hEx44-SAS | SaCas9-KKH |
| 1122384sh | CGATTTG | | | |
| hEx44-SaK- | AGGCGATTTGAC | 77 | hEx44-SAS | SaCas9-KKH |
| 11223961 | AGATCTGTTG | | | |
| hEx46-Sa- | TTCTCCAGGCTA | 78 | hEx46-SAS | SaCas9; SaCas9- |
| 1407225 | GAAGAACAA | | | KKH |
| hEx52-Sa- | TTGTTCTTACAG | 79 | hEx52-SAS | SaCas9; SaCas9- |
| 1609700 | GCAACAATG | | | KKH; SauriCas9; |
| | | | | SlugCas9 |
| hEx52-Sa- | TTACAGGCAACA | 80 | hEx52-SAS | SaCas9; SauriCas9; |
| 1609706 | ATGCAGGAT | | | SlugCas9 |
| hEx52-Sa- | TACAGGCAACAA | 81 | hEx52-SAS | SaCas9 |
| 1609707 | TGCAGGATT | | | |
| hEx52-SaK- | AAAAAACTTACT | 82 | hEx52-SDS | SaCas9-KKH |
| 1609805 | TCGATCCGT | | | |
| hEx52-SaK- | GTTAAAAAACTT | 83 | hEx52-SDS | SaCas9-KKH |
| 1609808 | ACTTCGATC | | | |
| hEx50-Sa- | TTAAAGAGGAAG | 84 | hEx50-SAS | SaCas9 |
| 1519371 | TTAGAAGAT | | | |
| hEx50-SaK- | TTTCTGTTAAAG | 85 | hEx50-SAS | SaCas9-KKH |
| 1519365 | AGGAAGTTA | | | |
| hEx50-SaK- | TACTTACAGGCT | 86 | hEx50-SDS | SaCas9-KKH |
| 1519457 | CCAATAGTG | | | |
| hEx50-Sau- | GTATACTTACAG | 87 | hEx50-SDS | SauriCas9; |
| 1519460 | GCTCCAATA | | | SlugCas9 |
| hEx50-SaK- | AGTATACTTACA | 88 | hEx50-SDS | SaCas9-KKH |
| 1519461 | GGCTCCAAT | | | |
| hEx50-SaK- | TCCAGTATACTT | 89 | hEx50-SDS | SaCas9-KKH |
| 1519464 | ACAGGCTCC | | | |
| hEx43-SaK- | TTTATATTACAG | 90 | hEx43-SAS | SaCas9-KKH |
| 1051747 | AATATAAAA | | | |

TABLE 4
Targeting sequences of exemplary
oligonucleotides for the utilization
of the prime editing technology
targeting human DMD exon 52

| | | SEQ |
|---|---|---|
| | | ID |
| Name | Sequence (5′→3′) | NO: |
| hEx52g4.PE.spacer | GTAATGAGTTCTTCCAACTG | 1 |
| hEx52g4.pbs13rt11 | CGTCCCCAGGTTTGGAAGAACTCA | 91 |
| hEx52g4.pbs13rt12 | GCGTCCCCAGGTTTGGAAGAACTCA | 92 |
| hEx52g4.pbs13rt13 | GGCGTCCCCAGGTTTGGAAGAACTC | 93 |
| | A | |
| hEx52g4.pbs13rt14 | AGGCGTCCCCAGGTTTGGAAGAACT | 94 |
| | CA | |
| hEx52g4.pbs13rt15 | GAGGCGTCCCCAGGTTTGGAAGAAC | 95 |
| | TCA | |
| hEx52g4.pbs13rt16 | AGAGGCGTCCCCAGGTTTGGAAGAA | 96 |
| | CTCA | |
| hEx52g4.pbs11rt15 | GAGGCGTCCCCAGGTTTGGAAGAAC | 97 |
| | T | |
| hE52g4.pbs12rt15 | GAGGCGTCCCCAGGTTTGGAAGAAC | 98 |
| | TC | |
| hE52g4.pbs14rt15 | GAGGCGTCCCCAGGTTTGGAAGAAC | 99 |
| | TCAT | |
| hE52g4.pbs15rt15 | GAGGCGTCCCCAGGTTTGGAAGAAC | 100 |
| | TCATT | |
| hE52g4.nick1 | GTTCTTACAGGCAACAATGC | 101 |
| hE52g4.nick2 | AAACAAGACCAGCAATCAAG | 102 |

In some embodiments, a nucleic acid may comprise one or more sequences encoding a gRNA. In some embodiments, a nucleic acid may comprise 1, 2, 3, 4, 5, 6, 7, 8, 9, 10, 11, 12, 13, 14, 15, 16, 17, 18, 19, 20, or 21 sequences encoding a gRNA. In some embodiments, all of the sequences encode the same gRNA. In some embodiments, all of the sequences encode different gRNAs. In some embodiments, at least 2 of the sequences encode the same gRNA, for example at least 3, 4, 5, 6, 7, 8, 9, 10, 11, 12, 13, 14, 15, 16, 17, 18, 19, 20 or 21 of the sequences encode the same gRNA.

In some embodiments, nucleotide gene editing may be performed in vitro or ex vivo. In some embodiments, cells are contacted in vitro or ex vivo with a nucleotide editing Cas9 and a gRNA that targets a dystrophin site. In some embodiments, the cells are contacted with one or more nucleic acids encoding the Cas9 and the guide RNA. In some embodiments, the one or more nucleic acids are introduced into the cells using, for example, lipofection or electroporation. Nucleotide gene editing may also be performed in zygotes. In embodiments, zygotes may be injected with one or more nucleic acids encoding Cas9 and a gRNA that targets a dystrophin site. The zygotes may subsequently be injected into a host.

In some embodiments, the Cas9 is provided on a vector. In embodiments, the vector contains a Cas9 derived from S. pyogenes (SpCas9). In some embodiments, the vector contains a Cas9 derived from S. aureus (SaCas9). In some embodiments, the vector contains a Cas9 derived from S. auricularis (SauCas9). In some embodiments, the vector contains a Cas9 derived from S. lugdunensis (SlugCas9). In some embodiments, the Cas9 sequence is codon optimized for expression in human cells or mouse cells. In some embodiments, the vector further contains a sequence encoding a fluorescent protein, such as GFP, which allows Cas 9-expressing cells to be sorted using fluorescence activated cell sorting (FACS). In some embodiments, the vector is a viral vector such as an adeno-associated viral vector.

In some embodiments, the gRNA is provided on a vector. In some embodiments, the vector is a viral vector such as an adeno-associated viral vector. In embodiments, the Cas9 and the guide RNA are provided on the same vector. In embodiments, the Cas9 and the guide RNA are provided on different vectors.

Any type of vector, such as any of those described herein, may be used. In some embodiments, the vector is a lipid nanoparticle. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector (AAV).

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of U.S. Patent Publication No. 2015/0111955, which is incorporated by reference herein in its entirety), AAV9 vector, AAV9P vector (also known as AAVMYO, see, Weinmann et al., 2020, Nature Communications, 11:5432), Myo-AAV vectors described in Tabebordbar et al., 2021, Cell, 184:1-20 (e.g., MyoAAV 1A, 2A, 3A, 4A, 4C, or 4E), and AAV9-rh74-HB-P1, AAV9-AAA-P1-SG vectors described in WO2022053630. wherein the number following AAV indicates the AAV serotype. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. In some embodiments, the vector is an AAV9 vector.

Efficiency of in vitro or ex vivo nucleotide editing Cas9 may be assessed using techniques known to those of skill in the art, such as the T7 E1 assay or sequencing. Restoration of DMD expression may be confirmed using techniques known to those of skill in the art, such as RT-PCR, Western blotting, and immunocytochemistry.

In some embodiments, in vitro or ex vivo gene editing is performed in a muscle or satellite cell. In some embodiments, gene editing is performed in iPSC or iCM cells. In embodiments, the iPSC cells are differentiated after gene editing. For example, the iPSC cells may be differentiated into a muscle cell or a satellite cell after editing. In embodiments, the iPSC cells are differentiated into cardiac muscle cells, skeletal muscle cells, or smooth muscle cells. In embodiments, the iPSC cells are differentiated into cardiomyocytes. iPSC cells may be induced to differentiate according to methods known to those of skill in the art.

In some embodiments, contacting the cell with the nucleotide editing Cas9 and the gRNA restores dystrophin expression. In embodiments, cells which have been edited in vitro or ex vivo, or cells derived therefrom, show levels of dystrophin protein that is comparable to wildtype cells. In embodiments, the edited cells, or cells derived therefrom, express dystrophin at a level that is 20%, 30%, 40%, 50%, 60%, 70%, 80%, 90%, 95% or any percentage in between of wildtype dystrophin expression levels.

### VI. Nucleic Acid Delivery

In some embodiments, expression cassettes are employed to express a transcription factor product, either for subsequent purification and delivery to a cell/subject, or for use directly in a genetic-based delivery approach. Provided herein are expression vectors which contain one or more nucleic acids encoding nucleotide editing Cas9 and at least one DMD guide RNA that targets a dystrophin site. In some embodiments, a nucleic acid encoding nucleotide editing Cas9 and a nucleic acid encoding at least one guide RNA are provided on the same vector. In further embodiments, a nucleic acid encoding nucleotide editing Cas9 and a nucleic acid encoding least one guide RNA are provided on separate vectors.

Expression requires that appropriate signals be provided in the vectors and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

In some embodiments, the nucleotide editing Cas9 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2b, HSP70, proliferin, tumor necrosis factor, and/or thyroid stimulating hormone a gene. In some embodiments, the inducer is phorbol ester (TFA), heavy metals, glucocorticoids, poly(rI)x, poly(rc), ElA, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are muscle specific promoters. These include the myosin light chain-2 promoter, the α-actin promoter, the troponin 1 promoter; the Na⁺/Ca²⁺ exchanger promoter, the dystrophin promoter, the α7 integrin promoter, the brain natriuretic peptide promoter and the αB-crystallin/small heat shock protein promoter, α-myosin heavy chain promoter and the ANF promoter. In some embodiments, the muscle specific promoter is the CK8 promoter. The CK8 promoter has the following sequence:

(SEQ ID NO: 103)
CTAGACTAGCATGCTGCCCATGTAAGGAGGCAAGGCCTGGGGACA
CCCGAGATGCCTGGTTATAATTAACCCAGACATGTGGCTGCCCCC
CCCCCCCCAACACCTGCTGCCTCTAAAAATAACCCTGCATGCCAT
GTTCCCGGCGAAGGGCCAGCTGTCCCCCGCCAGCTAGACTCAGCA
CTTAGTTTAGGAACCAGTGAGCAAGTCAGCCCTTGGGGCAGCCCA
TACAAGGCCATGGGGCTGGGCAAGCTGCACGCCTGGGTCCGGGGT
GGGCACGGTGCCCGGGCAACGAGCTGAAAGCTCATCTGCTCTCAG
GGGCCCCTCCCTGGGGACAGCCCCTCCTGGCTAGTCACACCCTGT
AGGCTCCTCTATATAACCCAGGGGCACAGGGGCTGCCCTCATTCT
ACCACCACCTCCACAGCACAGACAGACACTCAGGAGCCAGCCAGC

In some embodiments, the muscle-cell cell specific promoter is a variant of the CK8 promoter, called CK8e. The CK8e promoter has the following sequence:

(SEQ ID NO: 104)
TGCCCATGTAAGGAGGCAAGGCCTGGGGACACCCGAGATGCCTGG
TTATAATTAACCCAGACATGTGGCTGCCCCCCCCCCCCCAACACC
TGCTGCCTCTAAAAATAACCCTGCATGCCATGTTCCCGGCGAAGG
GCCAGCTGTCCCCCGCCAGCTAGACTCAGCACTTAGTTTAGGAAC
CAGTGAGCAAGTCAGCCCTTGGGGCAGCCCATACAAGGCCATGGG
GCTGGGCAAGCTGCACGCCTGGGTCCGGGGTGGGCACGGTGCCCG
GGCAACGAGCTGAAAGCTCATCTGCTCTCAGGGGCCCCTCCCTGG
GGACAGCCCCTCCTGGCTAGTCACACCCTGTAGGCTCCTCTATAT
AACCCAGGGGCACAGGGGCTGCCCTCATTCTACCACCACCTCCAC
AGCACAGACAGACACTCAGGAGCCAGCCAGC

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

B. 2A Peptide

In some embodiments, a 2A-like self-cleaving domain from the insect virus Thosea asigna (TaV 2A peptide) (EGRGSLLTCGDVEENPGP (SEQ ID NO: 105)) is used. These 2A-like domains have been shown to function across eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems has shown greater than 99% cleavage activity. Other acceptable 2A-like peptides include, but are not limited to, equine rhinitis A virus (ERAV) 2A peptide (QCTNYALLKLAGDVESNPGP (SEQ ID NO: 106)), porcine teschovirus-1 (PTV1) 2A peptide (ATNFSLLKQAGDVEENPGP (SEQ ID NO: 107)) and foot and mouth disease virus (FMDV) 2A peptide (PVKQLLNFDLLKLAGDVESNPGP (SEQ ID NO: 108)) or modified versions thereof.

In some embodiments, the 2A peptide is used to express a reporter and a nucleotide editing Cas9 simultaneously. The reporter may be, for example, GFP or mCherry.

Other self-cleaving peptides that may be used include but are not limited to nuclear inclusion protein a (Nia) protease, a P1 protease, a 3C protease, a L protease, a 3C-like protease, or modified versions thereof.

C. Trans-Splicing Inteins

In some embodiments, trans-splicing inteins are used to permit the covalent splicing of the split nucleotide editing Cas9. Due to delivery size limitation, nucleotide editing Cas9 can be split in N- and C-terminal peptides. Each half of the split nucleotide editing Cas9 when linked to trans-splicing inteins reassemble after translation into a functional nucleotide editing Cas9 that retains similar editing efficiencies compared to its non-split, full-length equivalent.

In some embodiments, the N- and C-terminal peptides of nucleotide editing Cas9 are fused to split DnaE intein halves from N. puntiforme (Npu).

Other trans-splicing inteins that may be used include but are not limited to Sce VMA, Mtu RecA, Ssp DnaE.

D. Delivery of Expression Vectors

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One method for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (ElA and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation. In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

The adenoviruses of the disclosure are replication defective, or at least conditionally replication defective. The adenovirus may be of any of the 42 different known serotypes or subgroups A-F. Adenovirus type 5 of subgroup C is the preferred starting material in order to obtain the conditional replication-defective adenovirus vector for use in the present disclosure.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes. Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination.

Other viral vectors may be employed as expression constructs in the present disclosure. Vectors derived from viruses such as vaccinia virus, adeno-associated virus (AAV) and herpesviruses may be employed. They offer several attractive features for various mammalian cells.

In embodiments, particular embodiments, the vector is an AAV vector. AAV is a small virus that infects humans and some other primate species. AAV is not currently known to cause disease. The virus causes a very mild immune response, lending further support to its apparent lack of pathogenicity. In many cases, AAV vectors integrate into the host cell genome, which can be important for certain applications, but can also have unwanted consequences. Gene therapy vectors using AAV can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. Recent human clinical trials using AAV for gene therapy in the retina have shown promise. AAV belongs to the genus Dependoparvovirus, which in turn belongs to the family Parvoviridae. The virus is a small (20 nm) replication-defective, nonenveloped virus.

Wild-type AAV has attracted considerable interest from gene therapy researchers due to a number of features. Chief amongst these is the virus's apparent lack of pathogenicity. It can also infect non-dividing cells and has the ability to stably integrate into the host cell genome at a specific site (designated AAVS1) in the human chromosome 19. This feature makes it somewhat more predictable than retroviruses, which present the threat of a random insertion and of mutagenesis, which is sometimes followed by development of a cancer. The AAV genome integrates most frequently into the site mentioned, while random incorporations into the genome take place with a negligible frequency. Development of AAVs as gene therapy vectors, however, has eliminated this integrative capacity by removal of the rep and cap from the DNA of the vector. The desired gene together with a promoter to drive transcription of the gene is inserted between the inverted terminal repeats (ITR) that aid in concatemer formation in the nucleus after the single-stranded vector DNA is converted by host cell DNA polymerase complexes into double-stranded DNA. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. This feature, along with the ability to infect quiescent cells present their dominance over adenoviruses as vectors for human gene therapy.

Use of the AAV does present some disadvantages. The cloning capacity of the vector is relatively limited and most therapeutic genes require the complete replacement of the virus's 4.8 kilobase genome. Large genes are, therefore, not suitable for use in a standard AAV vector. Options are currently being explored to overcome the limited coding capacity. The AAV ITRs of two genomes can anneal to form head to tail concatemers, almost doubling the capacity of the vector. Insertion of splice sites allows for the removal of the ITRs from the transcript.

Because of AAV's specialized gene therapy advantages, researchers have created an altered version of AAV termed self-complementary adeno-associated virus (scAAV). Whereas AAV packages a single strand of DNA and must wait for its second strand to be synthesized, scAAV packages two shorter strands that are complementary to each other. By avoiding second-strand synthesis, scAAV can express more quickly, although as a caveat, scAAV can only encode half of the already limited capacity of AAV. Recent reports suggest that scAAV vectors are more immunogenic than single stranded adenovirus vectors, inducing a stronger activation of cytotoxic T lymphocytes.

The humoral immunity instigated by infection with the wild type is thought to be a very common event. The associated neutralising activity limits the usefulness of the most commonly used serotype AAV2 in certain applications. Accordingly, the majority of clinical trials currently under way involve delivery of AAV2 into the brain, a relatively immunologically privileged organ. In the brain, AAV2 is strongly neuron-specific.

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

The Inverted Terminal Repeat (ITR) sequences comprise 145 bases each. They were named so because of their symmetry, which was shown to be required for efficient multiplication of the AAV genome. The feature of these sequences that gives them this property is their ability to form a hairpin, which contributes to so-called self-priming that allows primase-independent synthesis of the second DNA strand. The ITRs were also shown to be required for both integration of the AAV DNA into the host cell genome (19th chromosome in humans) and rescue from it, as well as for efficient encapsidation of the AAV DNA combined with generation of a fully assembled, deoxyribonuclease-resistant AAV particles.

With regard to gene therapy, ITRs seem to be the only sequences required in cis next to the therapeutic gene: structural (cap) and packaging (rep) proteins can be delivered in trans. With this assumption many methods were established for efficient production of recombinant AAV (rAAV) vectors containing a reporter or therapeutic gene. However, it was also published that the ITRs are not the only elements required in cis for the effective replication and encapsidation. A few research groups have identified a sequence designated cis-acting Rep-dependent element (CARE) inside the coding sequence of the rep gene. CARE was shown to augment the replication and encapsidation when present in cis.

On the “left side” of the genome there are two promoters called p5 and p19, from which two overlapping messenger ribonucleic acids (mRNAs) of different length can be produced. Each of these contains an intron which can be either spliced out or not. Given these possibilities, four various mRNAs, and consequently four various Rep proteins with overlapping sequence can be synthesized. Their names depict their sizes in kilodaltons (kDa): Rep78, Rep68, Rep52 and Rep40. Rep78 and 68 can specifically bind the hairpin formed by the ITR in the self-priming act and cleave at a specific region, designated terminal resolution site, within the hairpin. They were also shown to be necessary for the AAVS1-specific integration of the AAV genome. All four Rep proteins were shown to bind ATP and to possess helicase activity. It was also shown that they upregulate the transcription from the p40 promoter (mentioned below) but downregulate both p5 and p19 promoters.

The right side of a positive-sensed AAV genome encodes overlapping sequences of three capsid proteins, VP1, VP2 and VP3, which start from one promoter, designated p40. The molecular weights of these proteins are 87, 72 and 62 kiloDaltons, respectively. The AAV capsid is composed of a mixture of VP1, VP2, and VP3 totaling 60 monomers arranged in icosahedral symmetry in a ratio of 1:1:10, with an estimated size of 3.9 MegaDaltons.

The cap gene produces an additional, non-structural protein called the Assembly-Activating Protein (AAP). This protein is produced from ORF2 and is essential for the capsid-assembly process. The exact function of this protein in the assembly process and its structure have not been solved to date.

All three VPs are translated from one mRNA. After this mRNA is synthesized, it can be spliced in two different manners: either a longer or shorter intron can be excised resulting in the formation of two pools of mRNAs: a 2.3 kb- and a 2.6 kb-long mRNA pool. Usually, especially in the presence of adenovirus, the longer intron is preferred, so the 2.3-kb-long mRNA represents the so-called “major splice”. In this form the first AUG codon, from which the synthesis of VP1 protein starts, is cut out, resulting in a reduced overall level of VP1 protein synthesis. The first AUG codon that remains in the major splice is the initiation codon for VP3 protein. However, upstream of that codon in the same open reading frame lies an ACG sequence (encoding threonine) which is surrounded by an optimal Kozak context. This contributes to a low level of synthesis of VP2 protein, which is actually VP3 protein with additional N terminal residues, as is VP1.

Since the bigger intron is preferred to be spliced out, and since in the major splice the ACG codon is a much weaker translation initiation signal, the ratio at which the AAV structural proteins are synthesized in vivo is about 1:1:20, which is the same as in the mature virus particle. The unique fragment at the N terminus of VP1 protein was shown to possess the phospholipase A2 (PLA2) activity, which is probably required for the releasing of AAV particles from late endosomes. Muralidhar et al. reported that VP2 and VP3 are crucial for correct virion assembly. More recently, however, Warrington et al. showed VP2 to be unnecessary for the complete virus particle formation and an efficient infectivity, and also presented that VP2 can tolerate large insertions in its N terminus, while VP1 cannot, probably because of the PLA2 domain presence.

The AAV vector may be replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

In some embodiments, a single viral vector is used to deliver a nucleic acid encoding a nucleotide editing Cas9 and at least one gRNA to a cell. In some embodiments, nucleotide editing Cas9 is provided to a cell using a first viral vector and at least one gRNA is provided to the cell using a second viral vector. In some embodiment, the nucleotide editing Cas9 may use a split-intein dual AAV system which reconstitutes the full-length nucleotide editor by protein trans-splicing. In these systems, the Cas9 protein or the base editor is split into two sections, each fused with one part of an intein system (e.g., intein-N and intein-C encoded by dnaEn and dnaEc, respectively). Upon co-expression, the two sections of the Cas9 protein or nucleobase editor are ligated together via intein-mediated protein splicing. See, U.S. Pat. Publn. US20180127780, which is incorporated by reference herein in its entirety.

In some embodiments, a single viral vector is used to deliver a nucleic acid encoding nucleotide editing Cas9 and at least one gRNA to a cell. In some embodiments, nucleotide editing Cas9 is provided to a cell using a first viral vector and at least one gRNA is provided to the cell using a second viral vector. In some embodiment, the nucleotide editing Cas9 may use a split-intein dual AAV system which reconstitutes the full-length nucleotide editor by protein trans-splicing. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, triceps, extensor digitorum longus, diaphragm, or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement), or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ) to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

E. AAV-Cas9 Vectors

In some embodiments, a Cas9 base editor or prime editor may be packaged into an AAV vector. In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11 or any combination thereof.

Exemplary AAV-Cas9 vectors contain two ITR (inverted terminal repeat) sequences which flank a central sequence region comprising the Cas9 sequence. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-Cas9 vector may contain one or more nuclear localization signals (NLS). In some embodiments, the AAV-Cas9 vector contains 1, 2, 3, 4, or 5 nuclear localization signals. Exemplary NLS include the c-myc NLS, the SV40 NLS, the hnRNPAI M9 NLS, the nucleoplasmin NLS, the sequence RMRKFKNKGKDTAELRRRRVEVSVELRKAKKDEQILKRRNV (SEQ ID NO: 109) of the IBB domain from importin-alpha, the sequences VSRKRPRP (SEQ ID NO: 110) and PPKKARED (SEQ ID NO: 111) of the myoma T protein, the sequence PQPKKKPL (SEQ ID NO: 112) of human p53, the sequence SALIKKKKKMAP (SEQ ID NO: 113) of mouse c-abl IV, the sequences DRLRR (SEQ ID NO: 114) and PKQKKRK (SEQ ID NO: 115) of the influenza virus NS1, the sequence RKLKKKIKKL (SEQ ID NO: 116) of the Hepatitis virus delta antigen and the sequence REKKKFLKRR (SEQ ID NO: 117) of the mouse Mx1 protein. Further acceptable nuclear localization signals include bipartite nuclear localization sequences such as the sequence KRKGDEVDGVDEVAKKKSKK (SEQ ID NO: 118) of the human poly(ADP-ribose) polymerase or the sequence RKCLQAGMNLEARKTKK (SEQ ID NO: 119) of the steroid hormone receptors (human) glucocorticoid.

In some embodiments, the AAV-Cas9 vector may comprise additional elements to facilitate packaging of the vector and expression of the Cas9. In some embodiments, the AAV-Cas9 vector may comprise a polyA sequence. In some embodiments, the polyA sequence may be a mini-polyA sequence. In some embodiments, the AAV-CAs9 vector may comprise a transposable element. In some embodiments, the AAV-Cas9 vector may comprise a regulator element. In some embodiments, the regulator element is an activator or a repressor.

In some embodiments, the AAV-Cas9 may contain one or more promoters. In some embodiments, the one or more promoters drive expression of the Cas9. In some embodiments, the one or more promoters are muscle-specific promoters. Exemplary muscle-specific promoters include myosin light chain-2 promoter, the α-actin promoter, the troponin 1 promoter, the Na+/Ca2+ exchanger promoter, the dystrophin promoter, the α7 integrin promoter, the brain natriuretic peptide promoter, the αB-crystallin/small heat shock protein promoter, α-myosin heavy chain promoter, the ANF promoter, the CK8 promoter and the CK8e promoter.

In some embodiments, the AAV-Cas9 vector may be optimized for production in yeast, bacteria, insect cells, or mammalian cells. In some embodiments, the AAV-Cas9 vector may be optimized for expression in human cells. In some embodiments, the AAV-Cas9 vector may be optimized for expression in a bacculovirus expression system.

In some embodiments of the gene editing constructs of the disclosure, the construct comprises or consists of a promoter and a nuclease. In some embodiments, the construct comprises or consists of an CK8e promoter and a Cas9 nuclease. In some embodiments, the construct comprises or consists of an CK8e promoter and a Cas9 nuclease isolated or derived from Staphylococcus pyogenes (“SpCas9”). In some embodiments, the SpCas9 nuclease comprises or consists of a nucleotide sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to

(SEQ ID NO: 120)
GACAAGAAGTACAGCATCGGCCTGGACATCGGCACCAACTCTGTG
GGCTGGGCCGTGATCACCGACGAGTACAAGGTGCCCAGCAAGAAA
TTCAAGGTGCTGGGCAACACCGACCGGCACAGCATCAAGAAGAAC
CTGATCGGAGCCCTGCTGTTCGACAGCGGCGAAACAGCCGAGGCC
ACCCGGCTGAAGAGAACCGCCAGAAGAAGATACACCAGACGGAAG
AACCGGATCTGCTATCTGCAAGAGATCTTCAGCAACGAGATGGCC
AAGGTGGACGACAGCTTCTTCCACAGACTGGAAGAGTCCTTCCTG
GTGGAAGAGGATAAGAAGCACGAGCGGCACCCCATCTTCGGCAAC
ATCGTGGACGAGGTGGCCTACCACGAGAAGTACCCCACCATCTAC
CACCTGAGAAAGAAACTGGTGGACAGCACCGACAAGGCCGACCTG
CGGCTGATCTATCTGGCCCTGGCCCACATGATCAAGTTCCGGGGC
CACTTCCTGATCGAGGGCGACCTGAACCCCGACAACAGCGACGTG
GACAAGCTGTTCATCCAGCTGGTGCAGACCTACAACCAGCTGTTC
GAGGAAAACCCCATCAACGCCAGCGGCGTGGACGCCAAGGCCATC
CTGTCTGCCAGACTGAGCAAGAGCAGACGGCTGGAAAATCTGATC
GCCCAGCTGCCCGGCGAGAAGAAGAATGGCCTGTTCGGCAACCTG
ATTGCCCTGAGCCTGGGCCTGACCCCCAACTTCAAGAGCAACTTC
GACCTGGCCGAGGATGCCAAACTGCAGCTGAGCAAGGACACCTAC
GACGACGACCTGGACAACCTGCTGGCCCAGATCGGCGACCAGTAC
GCCGACCTGTTTCTGGCCGCCAAGAACCTGTCCGACGCCATCCTG
CTGAGCGACATCCTGAGAGTGAACACCGAGATCACCAAGGCCCCC
CTGAGCGCCTCTATGATCAAGAGATACGACGAGCACCACCAGGAC
CTGACCCTGCTGAAAGCTCTCGTGCGGCAGCAGCTGCCTGAGAAG
TACAAAGAGATTTTCTTCGACCAGAGCAAGAACGGCTACGCCGGC
TACATTGACGGCGGAGCCAGCCAGGAAGAGTTCTACAAGTTCATC
AAGCCCATCCTGGAAAAGATGGACGGCACCGAGGAACTGCTCGTG
AAGCTGAACAGAGAGGACCTGCTGCGGAAGCAGCGGACCTTCGAC
AACGGCAGCATCCCCCACCAGATCCACCTGGGAGAGCTGCACGCC
ATTCTGCGGCGGCAGGAAGATTTTTACCCATTCCTGAAGGACAAC
CGGGAAAAGATCGAGAAGATCCTGACCTTCCGCATCCCCTACTAC
GTGGGCCCTCTGGCCAGGGGAAACAGCAGATTCGCCTGGATGACC
AGAAAGAGCGAGGAAACCATCACCCCCTGGAACTTCGAGGAAGTG
GTGGACAAGGGCGCTTCCGCCCAGAGCTTCATCGAGCGGATGACC
AACTTCGATAAGAACCTGCCCAACGAGAAGGTGCTGCCCAAGCAC
AGCCTGCTGTACGAGTACTTCACCGTGTATAACGAGCTGACCAAA
GTGAAATACGTGACCGAGGGAATGAGAAAGCCCGCCTTCCTGAGC
GGCGAGCAGAAAAAGGCCATCGTGGACCTGCTGTTCAAGACCAAC
CGGAAAGTGACCGTGAAGCAGCTGAAAGAGGACTACTTCAAGAAA
ATCGAGTGCTTCGACTCCGTGGAAATCTCCGGCGTGGAAGATCGG
TTCAACGCCTCCCTGGGCACATACCACGATCTGCTGAAAATTATC
AAGGACAAGGACTTCCTGGACAATGAGGAAAACGAGGACATTCTG
GAAGATATCGTGCTGACCCTGACACTGTTTGAGGACAGAGAGATG
ATCGAGGAACGGCTGAAAACCTATGCCCACCTGTTCGACGACAAA
GTGATGAAGCAGCTGAAGCGGCGGAGATACACCGGCTGGGGCAGG
CTGAGCCGGAAGCTGATCAACGGCATCCGGGACAAGCAGTCCGGC
AAGACAATCCTGGATTTCCTGAAGTCCGACGGCTTCGCCAACAGA
AACTTCATGCAGCTGATCCACGACGACAGCCTGACCTTTAAAGAG
GACATCCAGAAAGCCCAGGTGTCCGGCCAGGGCGATAGCCTGCAC
GAGCACATTGCCAATCTGGCCGGCAGCCCCGCCATTAAGAAGGGC
ATCCTGCAGACAGTGAAGGTGGTGGACGAGCTCGTGAAAGTGATG
GGCCGGCACAAGCCCGAGAACATCGTGATCGAAATGGCCAGAGAG
AACCAGACCACCCAGAAGGGACAGAAGAACAGCCGCGAGAGAATG
AAGCGGATCGAAGAGGGCATCAAAGAGCTGGGCAGCCAGATCCTG
AAAGAACACCCCGTGGAAAACACCCAGCTGCAGAACGAGAAGCTG
TACCTGTACTACCTGCAGAATGGGCGGGATATGTACGTGGACCAG
GAACTGGACATCAACCGGCTGTCCGACTACGATGTGGACCATATC
GTGCCTCAGAGCTTTCTGAAGGACGACTCCATCGACAACAAGGTG
CTGACCAGAAGCGACAAGAACCGGGGCAAGAGCGACAACGTGCCC
TCCGAAGAGGTCGTGAAGAAGATGAAGAACTACTGGCGGCAGCTG
CTGAACGCCAAGCTGATTACCCAGAGAAAGTTCGACAATCTGACC
AAGGCCGAGAGAGGCGGCCTGAGCGAACTGGATAAGGCCGGCTTC
ATCAAGAGACAGCTGGTGGAAACCCGGCAGATCACAAAGCACGTG
GCACAGATCCTGGACTCCCGGATGAACACTAAGTACGACGAGAAT
GACAAGCTGATCCGGGAAGTGAAAGTGATCACCCTGAAGTCCAAG
CTGGTGTCCGATTTCCGGAAGGATTTCCAGTTTTACAAAGTGCGC
GAGATCAACAACTACCACCACGCCCACGACGCCTACCTGAACGCC
GTCGTGGGAACCGCCCTGATCAAAAAGTACCCTAAGCTGGAAAGC
GAGTTCGTGTACGGCGACTACAAGGTGTACGACGTGCGGAAGATG
ATCGCCAAGAGCGAGCAGGAAATCGGCAAGGCTACCGCCAAGTAC
TTCTTCTACAGCAACATCATGAACTTTTTCAAGACCGAGATTACC
CTGGCCAACGGCGAGATCCGGAAGCGGCCTCTGATCGAGACAAAC
GGCGAAACCGGGGAGATCGTGTGGGATAAGGGCCGGGATTTTGCC
ACCGTGCGGAAAGTGCTGAGCATGCCCCAAGTGAATATCGTGAAA
AAGACCGAGGTGCAGACAGGCGGCTTCAGCAAAGAGTCTATCCTG
CCCAAGAGGAACAGCGATAAGCTGATCGCCAGAAAGAAGGACTGG
GACCCTAAGAAGTACGGCGGCTTCGACAGCCCCACCGTGGCCTAT
TCTGTGCTGGTGGTGGCCAAAGTGGAAAAGGGCAAGTCCAAGAAA
CTGAAGAGTGTGAAAGAGCTGCTGGGGATCACCATCATGGAAAGA
AGCAGCTTCGAGAAGAATCCCATCGACTTTCTGGAAGCCAAGGGC
TACAAAGAAGTGAAAAAGGACCTGATCATCAAGCTGCCTAAGTAC
TCCCTGTTCGAGCTGGAAAACGGCCGGAAGAGAATGCTGGCCTCT
GCCGGCGAACTGCAGAAGGGAAACGAACTGGCCCTGCCCTCCAAA
TATGTGAACTTCCTGTACCTGGCCAGCCACTATGAGAAGCTGAAG
GGCTCCCCCGAGGATAATGAGCAGAAACAGCTGTTTGTGGAACAG
CACAAGCACTACCTGGACGAGATCATCGAGCAGATCAGCGAGTTC
TCCAAGAGAGTGATCCTGGCCGACGCTAATCTGGACAAAGTGCTG
TCCGCCTACAACAAGCACCGGGATAAGCCCATCAGAGAGCAGGCC
GAGAATATCATCCACCTGTTTACCCTGACCAATCTGGGAGCCCCT
GCCGCCTTCAAGTACTTTGACACCACCATCGACCGGAAGAGGTAC
ACCAGCACCAAAGAGGTGCTGGACGCCACCCTGATCCACCAGAGC
ATCACCGGCCTGTACGAGACACGGATCGACCTGTCTCAGCTGGGA
GGCGAC.

In some embodiments, the construct comprising a promoter and a nuclease further comprises at least two inverted terminal repeat (ITR) sequences. In some embodiments, the construct comprising a promoter and a nuclease further comprises at least two ITR sequences from isolated or derived from an AAV of serotype 2 (AAV2). In some embodiments, the construct comprising a promoter and a nuclease further comprises at least two ITR sequences each comprising or consisting of a nucleotide sequence of GGCCACTCCCTCTCTGCGCGCTCGCTCGCTCACTGAGGCCGGGCGACCAAAGGTCGCCCGAC GCCCGGGCTTTGCCCGGGCGGCCTCAGTGAGCGAGCGAGCGCGCAGAGAGGGA (SEQ ID NO: 121). In some embodiments, the construct comprising a promoter and a nuclease further comprises at least two ITR sequences, wherein the first ITR sequence comprises or consists of a nucleotide sequence of CCTGCAGGCAGCTGCGCGCTCGCTCGCTCACTGAGGCCGCCCGGGCAAAGCCCGGGCGTCGG GCGACCTTTGGTCGCCCGGCCTCAGTGAGCGAGCGAGCGCGCAGAGAGGGAGTGGCCAACTC CATCACTAGGGGTTCCT (SEQ ID NO: 122) and the second ITR sequence comprises or consist of a nucleotide sequence of AGGAACCCCTAGTGATGGAGTTGGCCACTCCCTCTCTGCGCGCTCGCTCGCTCACTGAGGCC GGGCGACCAAAGGTCGCCCGACGCCCGGGCTTTGCCCGGGCGGCCTCAGTGAGCGAGCGAGC GCGCAGCTGCCTGCAGG (SEQ ID NO: 123). In some embodiments, the construct comprises or consists of, from 5′ to 3′ a first ITR, a sequence encoding a promoter, a sequence encoding a nuclease and a second ITR. In some embodiments, the construct comprises or consists of, from 5′ to 3′ a first AAV2 ITR, a sequence encoding an CK8e promoter, a sequence encoding a SpCas9 nuclease and a second AAV2 ITR. In some embodiments, the construct comprising or consisting of, from 5′ to 3′ a first ITR, a sequence encoding a promoter, a sequence encoding a nuclease and a second ITR, further comprises a poly A sequence. In some embodiments, the polyA sequence comprises or consists of a minipolyA sequence. Exemplary minipolyA sequences of the disclosure comprise or consist of a nucleotide sequence of TAGCAATAAAGGATCGTTTATTTTCATTGGAAGCGTGTGTTGGTTTTTTGATCAGGCGCG (SEQ ID NO: 124). In some embodiments, the construct comprises or consists of, from 5′ to 3′ a first ITR, a sequence encoding a promoter, a sequence encoding a nuclease, a poly A sequence and a second ITR. In some embodiments, the construct comprises or consists of, from 5′ to 3′ a first ITR, a sequence encoding a promoter, a sequence encoding a nuclease, a minipoly A sequence and a second ITR. In some embodiments, the construct comprises or consists of, from 5′ to 3′ a first AAV2 ITR, a sequence encoding an CK8e promoter, a sequence encoding a SpCas9 nuclease, a minipoly A sequence and a second AAV2 ITR. In some embodiments, the construct comprising, from 5′ to 3′ a first ITR, a sequence encoding a promoter, a sequence encoding a nuclease, a poly A sequence and a second ITR, further comprises at least one nuclear localization signal. In some embodiments, the construct comprising, from 5′ to 3′ a first ITR, a sequence encoding a promoter, a sequence encoding a nuclease, a poly A sequence and a second ITR, further comprises at least two nuclear localization signals. Exemplary nuclear localization signals of the disclosure comprise or consist of a nucleotide sequence of AAGCGTCCTGCTGCTACTAAGAAAGCTGGTCAAGCTAAGAAAAAGAAA (SEQ ID NO: 125) or a nucleotide sequence of ATGGCCCCAAAGAAGAAGCGGAAGGTCGGTATCCACGGAGTCCCAGCAGCC (SEQ ID NO: 126). In some embodiments, the construct comprises or consists of, from 5′ to 3′ a first ITR, a sequence encoding a promoter, a sequence encoding a first nuclear localization signal, a sequence encoding a nuclease, a poly A sequence and a second ITR. In some embodiments, the construct comprises or consists of, from 5′ to 3′ a first ITR, a sequence encoding a promoter, a sequence encoding a first nuclear localization signal, a sequence encoding a nuclease, a sequence encoding a second nuclear localization signal, a poly A sequence and a second ITR. In some embodiments, the construct comprising, from 5′ to 3′ a first ITR, a sequence encoding a promoter, a sequence encoding a first nuclear localization signal, a sequence encoding a nuclease, a sequence encoding a second nuclear localization signal, a poly A sequence and a second ITR, further comprises a stop codon. The stop codon may have a sequence of TAG, TAA, or TGA. In some embodiments, the construct comprises or consists of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR. In some embodiments, the construct comprising or consisting of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR, further comprises transposable element inverted repeats. Exemplary transposable element inverted repeats of the disclosure comprise or consist of a nucleotide sequence of TGTGGGCGGACAAAATAGTTGGGAACTGGGAGGGGTGGAAATGGAGTTTTTAAGGATTATTT AGGGAAGAGTGACAAAATAGATGGGAACTGGGTGTAGCGTCGTAAGCTAATACGAAAATTAA AAATGACAAAATAGTTTGGAACTAGATTTCACTTATCTGGTT (SEQ ID NO: 127) and/or a nucleotide sequence of GAATATAGTCTTTACCATGCCCTTGGCCACGCCCCTCTTTAATACGACGGGCAATTTGCACT TCAGAAAATGAAGAGTTTGCTTTAGCCATAACAAAAGTCCAGTATGCTTTTTCACAGCATAA CTGGACTGATTTCAGTTTACAACTATTCTGTCTAGTTTAAGACTTTATTGTCATAGTTTAGA TCTATTTTGTTCAGTTTAAGACTTTATTGTCCGCCCACA (SEQ ID NO: 128). In some embodiments, the construct comprises or consists of, from 5′ to 3′ a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, and a second transposable element inverted repeat. In some embodiments, the construct comprising or consisting of, from 5′ to 3′, a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, and a second transposable element inverted repeat, further comprises a regulatory sequence. Exemplary regulatory sequences of the disclosure comprise or consist of a nucleotide sequence of CATGCAAGCTGTAGCCAACCACTAGAACTATAGCTAGAGTCCTGGGCGAACAAACGATGCTC GCCTTCCAGAAAACCGAGGATGCGAACCACTTCATCCGGGGTCAGCACCACCGGCAAGCGCC GCGACGGCCGAGGTCTTCCGATCTCCTGAAGCCAGGGCAGATCCGTGCACAGCACCTTGCCG TAGAAGAACAGCAAGGCCGCCAATGCCTGACGATGCGTGGAGACCGAAACCTTGCGCTCGTT CGCCAGCCAGGACAGAAATGCCTCGACTTCGCTGCTGCCCAAGGTTGCCGGGTGACGCACAC CGTGGAAACGGATGAAGGCACGAACCCAGTTGACATAAGCCTGTTCGGTTCGTAAACTGTAA TGCAAGTAGCGTATGCGCTCACGCAACTGGTCCAGAACCTTGACCGAACGCAGCGGTGGTAA CGGCGCAGTGGCGGTTTTCATGGCTTGTTATGACTGTTTTTTTGTACAGTCTATGCCTCGGG CATCCAAGCAGCAAGCGCGTTACGCCGTGGGTCGATGTTTGATGTTATGGAGCAGCAACGAT GTTACGCAGCAGCAACGATGTTACGCAGCAGGGCAGTCGCCCTAAAACAAAGTTAGGTGGCT CAAGTATGGGCATCATTCGCACATGTAGGCTCGGCCCTGACCAAGTCAAATCCATGCGGGCT GCTCTTGATCTTTTCGGTCGTGAGTTCGGAGACGTAGCCACCTACTCCCAACATCAGCCGGA CTCCGATTACCTCGGGAACTTGCTCCGTAGTAAGACATTCATCGCGCTTGCTGCCTTCGACC AAGAAGCGGTTGTTGGCGCTCTCGCGGCTTACGTTCTGCCCAAGTTTGAGCAGCCGCGTAGT GAGATCTATATCTATGATCTCGCAGTCTCCGGCGAGCACCGGAGGCAGGGCATTGCCACCGC GCTCATCAATCTCCTCAAGCATGAGGCCAACGCGCTTGGTGCTTATGTGATCTACGTGCAAG CAGATTACGGTGACGATCCCGCAGTGGCTCTCTATACAAAGTTGGGCATACGGGAAGAAGTG ATGCACTTTGATATCGACCCAAGTACCGCCACCTAACAATTCGTTCAAGCCGAGATCGGCTT CCCGGCCGCGGAGTTGTTCGGTAAATTGTCACAACGCCG (SEQ ID NO: 129). In some embodiments, the construct comprises or consists of, from 5′ to 3′ a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, a regulatory sequence and a second transposable element inverted repeat. In some embodiments, the construct may further comprise one or more spacer sequences. Exemplary spacer sequences of the disclosure have length from 1-1500 nucleotides, inclusive of all ranges therebetween. In some embodiments, the spacer sequences may be located either 5′ to or 3′ to an ITR, a promoter, a nuclear localization sequence, a nuclease, a stop codon, a polyA sequence, a transposable element inverted repeat, and/or a regulator element.

F. AAV-sgRNA Vectors

In some embodiments, at least a first sequence encoding a gRNA and a second sequence encoding a gRNA may be packaged into an AAV vector. In some embodiments, at least a first sequence encoding a gRNA, a second sequence encoding a gRNA, and a third sequence encoding a gRNA may be packaged into an AAV vector. In some embodiments, at least a first sequence encoding a gRNA, a second sequence encoding a gRNA, a third sequence encoding a gRNA, and a fourth sequence encoding a gRNA may be packaged into an AAV vector. In some embodiments, at least a first sequence encoding a gRNA, a second sequence encoding a gRNA, a third sequence encoding a gRNA, a fourth sequence encoding a gRNA, and a fifth sequence encoding a gRNA may be packaged into an AAV vector. In some embodiments, a plurality of sequences encoding a gRNA are packaged into an AAV vector. For example, 1, 2, 3, 4, 5, 6, 7, 8, 9, 10, 11, 12, 13, 14, 15, 16, 17, 18, 19, or 20 sequences encoding a gRNA may be packaged into an AAV vector. In some embodiments, each sequence encoding a gRNA is different. In some embodiments, at least 1, 2, 3, 4, 5, 6, 7, 8, 9, 10, 11, 12, 13, 14, 15, 16, 17, 18, 19, or 20 of the sequences encoding a gRNA are the same. In some embodiments, all of the sequence encoding a gRNA are the same.

In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11 or any combination thereof.

Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs are isolated or derived from an AAV vector of a first serotype and a sequence encoding a capsid protein of the AAV-sgRNA vector is isolated or derived from an AAV vector of a second serotype. In some embodiments, the first serotype and the second serotype are the same. In some embodiments, the first serotype and the second serotype are not the same. In some embodiments, the first serotype is AAV1, AAV2, AAV3, AAV4, AAV5, AAV6, AAV7, AAV8, AAV9, AAV10, or AAV11. In some embodiments, the second serotype is AAV1, AAV2, AAV3, AAV4, AAV5, AAV6, AAV7, AAV8, AAV9, AAV10, or AAV11. In some embodiments, the first serotype is AAV2 and the second serotype is AAV1, AAV2, AAV3, AAV4, AAV5, AAV6, AAV7, AAV8, AAV9, AAV10, or AAV11. In some embodiments, the first serotype is AAV2 and the second serotype is AAV9.

Exemplary AAV-sgRNA vectors contain two ITR (inverted terminal repeat) sequences which flank a central sequence region comprising the gRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, a first ITR is isolated or derived from an AAV vector of a first serotype, a second ITR is isolated or derived from an AAV vector of a second serotype and a sequence encoding a capsid protein of the AAV-sgRNA vector is isolated or derived from an AAV vector of a third serotype. In some embodiments, the first serotype and the second serotype are the same. In some embodiments, the first serotype and the second serotype are not the same. In some embodiments, the first serotype, the second serotype, and the third serotype are the same. In some embodiments, the first serotype, the second serotype, and the third serotype are not the same. In some embodiments, the first serotype is AAV1, AAV2, AAV3, AAV4, AAV5, AAV6, AAV7, AAV8, AAV9, AAV10, or AAV11. In some embodiments, the second serotype is AAV1, AAV2, AAV3, AAV4, AAV5, AAV6, AAV7, AAV8, AAV9, AAV10, or AAV11. In some embodiments, the third serotype is AAV1, AAV2, AAV3, AAV4, AAV5, AAV6, AAV7, AAV8, AAV9, AAV10, or AAV11. In some embodiments, the first serotype is AAV2, the second serotype is AAV4 and the third serotype is AAV1, AAV2, AAV3, AAV4, AAV5, AAV6, AAV7, AAV8, AAV9, AAV10, or AAV11. In some embodiments, the first serotype is AAV2, the second serotype is AAV4 and the third serotype is AAV9. Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-sgRNA vector may comprise additional elements to facilitate packaging of the vector and expression of the sgRNA. In some embodiments, the AAV-sgRNA vector may comprise a transposable element. In some embodiments, the AAV-sgRNA vector may comprise a regulatory element. In some embodiments, the regulatory element comprises an activator or a repressor. In some embodiments, the AAV-sgRNA sequence may comprise a non-functional or “stuffer” sequence. Exemplary stuffer sequences of the disclosure may have some (a non-zero percentage of) identity or homology to a genomic sequence of a mammal (including a human). Alternatively, exemplary stuffer sequences of the disclosure may have no identify or homology to a genomic sequence of a mammal (including a human). Exemplary stuffer sequences of the disclosure may comprise or consist of naturally occurring non-coding sequences or sequences that are neither transcribed nor translated following administration of the AAV vector to a subject.

In some embodiments, the AAV-sgRNA vector may be optimized for production in yeast, bacteria, insect cells, or mammalian cells. In some embodiments, the AAV-sgRNA vector may be optimized for expression in human cells. In some embodiments, the AAV-Cas9 vector may be optimized for expression in a bacculovirus expression system.

In some embodiments, the AAV-sgRNA vector comprises at least one promoter. In some embodiments, the AAV-sgRNA vector comprises at least two promoters. In some embodiments, the AAV-sgRNA vector comprises at least three promoters. In some embodiments, the AAV-sgRNA vector comprises at least four promoters. In some embodiments, the AAV-sgRNA vector comprises at least five promoters. Exemplary promoters include, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus. Further exemplary promoters include the U6 promoter, the H1 promoter, and the 7SK promoter.

In some embodiments, the AAV vector comprises a first sequence encoding a gRNA and a second sequence encoding a gRNA, a first promoter drives expression of the first sequence encoding a gRNA and a second promoter drives expression of the second sequence encoding a gRNA. In some embodiments, the first and second promoters are the same. In some embodiments, the first and second promoters are different. In some embodiments, the first and second promoters are selected from the H1 promoter, the U6 promoter, and the 7SK promoter. In some embodiments, the first sequence encoding a gRNA and the second sequence encoding a gRNA are identical. In some embodiments, the first sequence encoding a gRNA and the second sequence encoding a gRNA are not identical.

In some embodiments, the AAV vector comprises a first sequence encoding a gRNA, a second sequence encoding a gRNA, and a third sequence encoding a gRNA, a first promoter drives expression of the first sequence encoding a gRNA, a second promoter drives expression of the second sequence encoding a gRNA, and a third promoter drives expression of a third sequence encoding a gRNA. In some embodiments, at least two of the first, second, and third promoters are the same. In some embodiments, each of the first, second, and third promoters are different. In some embodiments, the first, second, and third promoters are selected from the H1 promoter, the U6 promoter, and the 7SK promoter. In some embodiments, the first promoter is the U6 promoter. In some embodiments, the second promoter is the H1 promoter. In some embodiments, the third promoter is the 7SK promoter. In some embodiments, the first promoter is the U6 promoter, the second promoter is the H1 promoter, and the third promoter is the 7SK promoter. In some embodiments, the first sequence encoding a gRNA, the second sequence encoding a gRNA, and the third sequence encoding a gRNA are identical. In some embodiments, the first sequence encoding a gRNA, the second sequence encoding a gRNA, and the third sequence encoding a gRNA are not identical.

In some embodiments, the AAV vector comprises a first sequence encoding a gRNA, a second sequence encoding a gRNA, a third sequence encoding a gRNA, and a fourth sequence encoding a gRNA, a first promoter drives expression of the first sequence encoding a gRNA, a second promoter drives expression of the second sequence encoding a gRNA, a third promoter drives expression of the third sequence encoding a gRNA, and a fourth promoter drives expression of the fourth sequence encoding a gRNA. In some embodiments, at least two of the first, second, third, and fourth promoters are the same. In some embodiments, each of the first, second, third, and fourth promoters are different. In some embodiments, each of the first, second, third and fourth promoters are selected from the H1 promoter, the U6 promoter, and the 7SK promoter. In some embodiments, the first sequence encoding a gRNA, the second sequence encoding a gRNA, the third sequence encoding a gRNA, and the fourth sequence encoding a gRNA are identical. In some embodiments, the first sequence encoding a gRNA, the second sequence encoding a gRNA, the third sequence encoding a gRNA, and the fourth sequence encoding a gRNA are not identical.

In some embodiments, the AAV vector comprises a first sequence encoding a gRNA, a second sequence encoding a gRNA, a third sequence encoding a gRNA, a fourth sequence encoding a gRNA, and a fifth sequence encoding a gRNA, a first promoter drives expression of the first sequence encoding a gRNA, a second promoter drives expression of the second sequence encoding a gRNA, a third promoter drives expression of the third sequence encoding a gRNA, a fourth promoter drives expression of the fourth sequence encoding a gRNA, and a fifth promoter drives expression of the fifth sequence encoding a gRNA. In some embodiments, at least two of the first, second, third, fourth, and fifth promoters are the same. In some embodiments, each of the first, second, third, fourth, and fifth promoters are different. In some embodiments, each of the first, second, third, and fourth promoters are different. In some embodiments, each of the first, second, third, fourth and fifth promoters are selected from the H1 promoter, the U6 promoter, and the 7SK promoter. In some embodiments, the first sequence encoding a gRNA, the second sequence encoding a gRNA, the third sequence encoding a gRNA, the fourth sequence encoding a gRNA, and the fifth sequence encoding a gRNA are identical. In some embodiments, the first sequence encoding a gRNA, the second sequence encoding a gRNA, the third sequence encoding a gRNA, the fourth sequence encoding a gRNA, and the fifth sequence encoding a gRNA are not identical.

### VII. Pharmaceutical Compositions and Delivery Methods

For clinical applications, pharmaceutical compositions are prepared in a form appropriate for the intended application. Generally, this entails preparing compositions that are essentially free of pyrogens, as well as other impurities that could be harmful to humans or animals.

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients of the present disclosure, its use in therapeutic compositions may be used. Supplementary active ingredients also can be incorporated into the compositions, provided they do not inactivate the vectors or cells of the compositions.

In some embodiments, the active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine) and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the nucleotide editing Cas9 and gRNAs described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding nucleotide editing Cas9 and a guide RNA that targets a dystrophin splice site are provided to a cell ex vivo before the cell is introduced or reintroduced to a patient.

### VIII. Definitions

The term “nucleotide editing Cas9” refers to a Cas9 protein fused to a base editor or a prime editor. Non-limiting examples of Cas9 include SpCas9, SpCas9-NG, SaCas9, SaCas9-KKH, SauCas9, and SlugCas9. Non limiting examples of a base editor include ABEmax, ABE8e, ABE8eV106W, ABE8.20-m.

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

As used herein, “AAV” refers to an adeno-associated virus vector. As used herein, “AAV” refers to any AAV serotype and variant, including but not limited to an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), AAV9 vector, AAV9P vector (also known as AAVMYO, see, Weinmann et al., 2020, Nature Communications, 11:5432), and Myo-AAV vectors described in Tabebordbar et al., 2021, Cell, 184:1-20 (e.g., MyoAAV 1A, 2A, 3A, 4A, 4C, or 4E), wherein the number following AAV indicates the AAV serotype. The term “AAV” can also refer to any known AAV (vector) system. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. Structurally, AAVs are small (25 nm), single-DNA stranded non-enveloped viruses with an icosahedral capsid. Naturally occurring or engineered AAV serotypes and variants that differ in the composition and structure of their capsid protein have varying tropism, i.e., ability to transduce different cell types. When combined with active promoters, this tropism defines the site of gene expression.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

Target sequences for Cas9s include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′) and may be capable of functioning even when positioned either upstream or downstream of the promoter.

Promoters and/or enhancers may be derived in their entirety from a native gene or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. For clarity, the terms “spacer sequence”, “spacer,” “protospacer,” “guide sequence,” or “targeting sequence” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the disclosure, such polypeptides encoded by nucleic acid sequences are not required to be identical to the endogenous protein that is defective, or whose activity, function, or expression is insufficient, deficient or absent in a treated mammal.

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors or nanoparticles to various organs and tissues.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the disclosure will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this disclosure, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

### IX. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

Study design. This study aimed to use nucleotide editing technologies to correct the DMD-causing ΔEx51 deletion mutation in the DMD gene in both a mouse model and a human iPSC model of DMD. This resulted in rescue of dystrophin protein expression, improvement of skeletal muscle fiber architecture in vivo, and reduction of arrhythmic cardiomyocytes in human iPSCs. Mouse sgRNAs were tested in vitro for base editing at the target splice acceptor or donor sites of exon 50 or 52, and genome editing efficiencies were evaluated using Sanger sequencing. AAV9 was used to deliver, in vivo, the sgRNA with highest efficiency and an adenine base editor by intramuscular injection. The editing outcomes were evaluated using Sanger sequencing, RT-PCR, Western blot analysis, immunohistochemistry, and H&E staining. Mice injected with saline solution served as control. As an additional control, one leg of the mouse was injected with saline solution and the other leg with AAV containing the base editing components. Human sgRNAs were tested in vitro for gene correction by base editing or prime editing. The optimal sgRNAs were nucleofected into iPSCs with the ΔEx51 mutation. Editing outcomes were evaluated in iPSC-derived cardiomyocytes by Sanger sequencing, RT-PCR, Western blot analysis, immunocytochemistry, and calcium imaging. Each experiment was conducted in replicate as indicated by n values in the figure legends. Sample size was chosen to use the fewest number of animals to achieve statistical significance; no statistical methods were used to predetermine sample size. All experimental samples were included in the analyses, with no data excluded.

Study approval. All experimental procedures involving animals in this study were reviewed and approved by the University of Texas Southwestern Medical Center's Institutional Animal Care and Use Committee.

Plasmids and cloning. The pmCherry_gRNA plasmid contained a U6-driven sgRNA scaffold and a CMV-driven pmCherry fluorescent protein. pmCherry_gRNA was a gift from Ervin Welker (Addgene plasmid #80457). pCMV_ABEmax_P2A_GFP (Addgene plasmid #112101) (Koblan et al., 2018), NG-ABEmax (Addgene plasmid #124163) (Huang et al., 2019), pCMV-PE2-P2A-GFP (Addgene plasmid #132776) (Anzalone et al., 2019), pU6-pegRNA-GG-acceptor (Addgene plasmid #132777) (Anzalone et al., 2019), ABE8e (Addgene plasmid #138489), and NG-ABE8e (Addgene plasmid #138491) were gifts from David Liu. The N-term ABE and C-term ABE constructs were adapted from Cbh_v5 AAV-ABE N-terminal (Addgene plasmid #137177) (Levy et al., 2020) and Cbh_v5 AAV-ABE C-terminal (Addgene plasmid #137178) (Levy et al., 2020) and synthesized by Twist Biotechnologies and GenScript. The pSpCas9(BB)-2A-GFP (PX458) plasmid used for the generation of isogenic ΔEx51 iPSCs was a gift from F. Zhang (Addgene plasmid #48138) (Ran et al., 2013). Cloning of sgRNAs was done using NEBuilder HiFi DNA Assembly (NEB) into restriction enzyme-digested destination vectors.

Cell culture and transfection. N2a and 293T cells were maintained in DMEM supplemented with 10% (v/v) fetal bovine serum. For transfection experiments, cells were seeded onto 24-well plates at 125,000 cells per well. The following day, cells were transfected by Lipofectamine 2000 (Thermo Fisher Scientific), according to the manufacturer's instructions. Cells were harvested for downstream analyses three days later. The sequences of the tested sgRNAs are listed in Table 5.

Sequencing analysis. Genomic DNA of mouse N2a cells, human 293T cells, and human iPSCs was isolated using DirectPCR cell lysis reagent (Viagen) supplemented with 1 μg/μL of Proteinase K according to the manufacturer's protocol. Genomic DNA of mouse muscle tissues was isolated using the DNeasy Blood and Tissue Kit (QIAGEN) according to the manufacturer's protocol. Total RNA of mouse skeletal muscles, and human iPSC derived cardiomyocytes was isolated using RNeasy Mini Kit (QIAGEN) according to the manufacturer's protocol. cDNA was reverse transcribed from total RNA using iScript cDNA Synthesis Kit (Bio-Rad). Genomic DNA and cDNA were PCR amplified using PrimeStar GXL DNA Polymerase (Takara). Top 8 potential off-target sites were predicted by CRISPOR (Concordet & Haeussler, 2018). Base editing on-target and off-target efficiencies were analyzed from Sanger sequencing by EditR (Kluesner et al., 2018). Prime editing efficiency was analyzed from Sanger sequencing by TIDE analysis (Brinkman et al., 2014). Primers of the PCR reactions are listed in Table 5.

TABLE 5
sgRNA targeting sequences and primer sequences used in the Examples 2-9

| Purpose | ID | Sequence (5′-3′) | SEQ ID NO: |
|---|---|---|---|
| sgRNAs | mEx50-sgRNA-1 | CTATTAAAGAGGAAGTTAGA | 130 |
| targeting mouse | | | |
| exon 50 splice | | | |
| sites | | | |
| | mEx50-sgRNA-2 | AAAGAGGAAGTTAGAAGATC | 131 |
| | mEx50-sgRNA-3 | TACAGGCTCCAGTAGTGCTC | 132 |
| | mEx50-sgRNA-4 | GTACTTACAGGCTCCAGTAG | 133 |
| | mEx50-sgRNA-5 | ATGTACTTACAGGCTCCAGT | 134 |
| sgRNAs | mEx52-sgRNA-1 | TTCTTTCAGGCAACACTGCA | 135 |
| targeting mouse | | | |
| exon 52 splice | | | |
| sites | | | |
| | mEx52-sgRNA-2 | TCAGGCAACACTGCAAGATT | 136 |
| | mEx52-sgRNA-3 | TTACTTCGATCAGTAATGAT | 137 |
| | mEx52-sgRNA-4 | AGACTTACTTCGATCAGTAA | 138 |
| sgRNA | hEx50-sgRNA-1 | ATACTTACAGGCTCCAATAG | 139 |
| targeting | | | |
| humanexon 50 | | | |
| splice sites | | | |
| sgRNAs | hEx52-sgRNA-2 | GTTCTTACAGGCAACAATGC | 140 |
| targeting | | | |
| humanexon 52 | | | |
| splice sites | | | |
| | hEx52-sgRNA-3 | ACAGGCAACAATGCAGGATT | 141 |
| sgRNA | hEx51-1565260 | ATTTTAGCTCCTACTCAGAC | 142 |
| targeting | | | |
| humanexon 51 | | | |
| splice site | | | |
| | hEx51-1565475 | TTCTCATACCTTCTGCTTGA | 143 |
| sgRNA | hEx45-1370936 | ATCTTACAGGAACTCCAGGA | 144 |
| targeting | | | |
| humanexon 45 | | | |
| splice site | | | |
| | hEx45-1370937 | TCTTACAGGAACTCCAGGAT | 145 |
| | hEx45-1370942 | CAGGAACTCCAGGATGGCAT | 146 |
| | hEx45-1370943 | AGGAACTCCAGGATGGCATT | 147 |
| | hEx45-1371093 | CCCTACCTCTTTTTTCTGTC | 148 |
| Primers for | mEx50-F | CTGAAATGATGGCCAAGGAT | 149 |
| Sanger | | | |
| sequencing | | | |
| analyses | | | |
| | mEx50-R | CCACAAAGCCATCCAGTTTT | 150 |
| | mEx52-F | AACTGGAGATTCATATTGTTATC | 151 |
| | | CTG | |
| | mEx52-R | GCCCTACCTTCATGAAGATTACA | 152 |
| | | AC | |
| | hEx50-F | ATGCCTGGAGAAAGGGTTTT | 153 |
| | hEx50-R | CTCTCCCACAATCTCCCTTG | 154 |
| | hEx52-F | GAATGAAACATATTTCCTGTTAA | 155 |
| | | ATTG | |
| | hEx52-R | GAAAGATGGCCCAGGAAGA | 156 |
| | m51cDNA-F | AGGTTCACTTAAAAGATTTTAGG | 157 |
| | | CAG | |
| | m51cDNA-R | TAACATTTCATTCAACTGTTGTC | 158 |
| | | TCC | |
| | h51cDNA-F | TAAAAGACCTTGGGCAGCTT | 159 |
| | h51cDNA-R | GCCAACTGCTTGGTTTCTGT | 160 |
| Primers | OFF-1-mABED50-F | CGTCTGAATGCGCTTGTCTG | 161 |
| for off- | | | |
| target | | | |
| analysis | | | |
| | OFF-1-mABED50-R | ACAGCCGTCCAATGTCAACT | 162 |
| | OFF-2-mABED50-F | CAAGGGTTCCTCGACTGCAT | 163 |
| | OFF-2-mABED50-R | TGTACATGGAAGCTGGCCAC | 164 |
| | OFF-3-mABED50-F | AGGCTGCTGGAGACTTAGGA | 165 |
| | OFF-3-mABED50-R | CTAGAGAGCCAGGTGGTCCT | 166 |
| | OFF-4-mABED50-F | CCTCCAGCAACTTAGGGACC | 167 |
| | OFF-4-mABED50-R | CCTTCTCCCTGTCTGCCTTG | 168 |
| | OFF-5-mABED50-F | CTGTGGAAAGCGGAGACACT | 169 |
| | OFF-5-mABED50-R | CCAACCTTCTCAGGGCAGTT | 170 |
| | OFF-6-mABED50-F | GAGGACAACTGGAAGGCCTG | 171 |
| | OFF-6-mABED50-R | TCTGTCTGCCAGAGTTGTTTTCT | 172 |

AAV vector production. AAVs were prepared by the Boston Children's Hospital Viral Core, as previously described (Brinkman et al., 2014). AAV vectors were purified by discontinuous iodixanol gradients (Cosmo Bio, AXS-1114542-5) and concentrated with a Millipore Amicon filter unit (UFC910008, 100 kDa). AAV titers were determined by quantitative real-time PCR assays.

Mice. Mice were housed in a barrier facility with a 12-h:12-h light:dark cycle and maintained on standard chow (2916 Teklad Global). ΔEx51 mice and WT littermates were genotyped as previously described (Chemello et al., 2020). All experiments used only male mice. Animals were assigned to experimental groups by genotype. No exclusion, randomization, or blinding approaches were used to assign animals for experiments. All AAV injections and dissections were conducted in an unblinded fashion.

AAV9 delivery to ΔEx51 mice. Prior to intramuscular injections, mice were anesthetized by intraperitoneal injection of a ketamine and xylazine anesthetic cocktail. Intramuscular injection of P12 male ΔEx51 mice was performed via slow longitudinal injection into TA muscles using an ultrafine needle (31G) with 50 μL of saline solution or a prepared mixture of the dual AAV9 viruses (5×10¹⁰ vg/leg of each virus).

Western blot analysis. Western blot analyses were performed as previously described (Min et al., 2020). Briefly, for Western blots of muscles, tissues were crushed using a liquid-nitrogen-frozen crushing apparatus. For Western blots of iPSC-derived cardiomyocytes, 2×10⁶ cardiomyocytes were harvested and lysed in lysis buffer (10% SDS, 62.5 mM Tris [pH 6.8], 1 mM EDTA, and protease inhibitor) with a pestle. Cell or tissue lysates were passed through a 25G syringe and then a 27G syringe, 10 times each. 50 μg of total protein was loaded onto a 4%-20% acrylamide gel. Blots were then incubated with mouse anti-dystrophin antibody (Sigma-Aldrich, D8168) at 4° C. overnight for dystrophin detection or with anti-vinculin antibody (Sigma-Aldrich, V9131) at room temperature for 1 h for vinculin detection (loading control), and then with horseradish peroxidase (HRP) antibody (Bio-Rad Laboratories) at room temperature for 1 h. Blots were developed using Western Blotting Luminol Reagent (Santa Cruz Biotechnology, sc-2048).

Histological analyses of skeletal muscles. Muscles were individually dissected and cryo-embedded in a 1:2 volume mixture of Gum Tragacanth powder (Sigma-Aldrich) to Tissue Freezing Medium (TFM) (Triangle Bioscience). All embeds were snap frozen in isopentane heat extractant and supercooled to −155° C. Eight-micron transverse sections of muscle were prepared on a Leica CM3050 cryostat. H&E staining was performed as previously described in established staining protocols (Long et al., 2016). Dystrophin immunohistochemistry was performed using MANDYS8 monoclonal antibody (Sigma-Aldrich, D8168) with modifications to manufacturer's instructions as previously described (Min et al., 2020). Image analyses were performed using Fiji software (Schneider et al., 2012) on at least three muscles for each condition as indicated in the figures. Myofiber diameter was calculated as minimal Feret's diameter, a geometrical parameter for reliable measurement of cross-sectional size (Briguet et al., 2004).

Human iPSC maintenance and nucleofection. Human iPSCs were cultured on Matrigel-coated polystyrene tissue culture plates and maintained in mTeSR Plus media (Stem Cell Technologies). Cells were passaged at 60-80% confluence using Versene (GIBCO). One hour before nucleofection, iPSCs were treated with 10 μM ROCK inhibitor, Y-27632 (Selleckchem). iPSCs were then dissociated into single cells using Accutase (Innovative Cell Technologies). For the base editing studies, iPSCs (8×10⁵) were mixed with 1.5 μg of pmCherry_gRNA plasmid containing the target sgRNA and 4.5 μg of pCMV_ABEmax_P2A_GFP. For the prime editing studies, iPSCs (8×10⁵) were mixed with 500 ng of pmCherry_gRNA plasmid containing the nicking sgRNA, 1.5 μg of the pU6-pegRNA-GG-acceptor plasmid containing the target pegRNA, and 4.5 μg of the pCMV-PE2-P2A-GFP plasmid. iPSCs were then nucleofected using the P3 Primary Cell 4D-Nucleofector X Kit (Lonza) according to the manufacturer's protocol. After nucleofection, iPSCs were cultured in mTeSR Plus media supplemented with 10 μM ROCK inhibitor and 100 μg/mL Primocin (InvivoGen), and then switched to fresh mTeSR Plus media the following day. Three days after nucleofection, GFP and pmCherry double-positive cells were isolated by fluorescence-activated cell sorting. Mixed population or single clones were isolated, expanded, genotyped, and sequenced.

Human iPSC-cardiomyocyte differentiation. Human iPSCs at 60-80% confluency were differentiated into cardiomyocytes as previously described (Burridge et al., 2014). Briefly, cells were cultured in CDM3 media supplemented with 4-6 μM CHIR99021 (Selleckchem) for 48 hours (days 1-2), and then CDM3 media supplemented with 2 μM WNT-C59 (Selleckchem) for 48 hours (days 3-4). Starting on day 5, cells were cultured in basal media (RPMI-1640, GIBCO, supplemented with B-27 Supplement, Thermo Scientific) for 6 days (days 5-10). On day 10, cells were cultured in selective media (RPMI-1640, without glucose, GIBCO, supplemented with B-27 Supplement) for 10 days (days 11-20), and then basal media thereafter. Cardiomyocytes were used for experiments on day 30 and harvested using TrypLE Express (Thermo Scientific).

Human iPSC-cardiomyocyte immunocytochemistry. Dystrophin and troponin-I immunocytochemistry of iPSC-derived cardiomyocytes was performed as previously described (Kyrychenko et al., 2017). Briefly, iPSC-derived cardiomyocytes (1×105) were seeded on 12 mm coverslips coated with poly-D-lysine and Matrigel (Corning) and fixed in cold acetone (10 minutes, −20° C.). Following fixation, coverslips were equilibrated in PBS, and then blocked for 1 hour with serum cocktail (2% normal horse serum/2% normal donkey serum/0.2% BSA/PBS). Mouse anti-dystrophin (1:800) (MANDYS8, Sigma-Aldrich, D8168), and rabbit anti-troponin-I (1:200) (clone H170, Santa Cruz Biotechnology, sc-15368) in 0.2% BSA/PBS were applied and incubated overnight at 4° C. Then, coverslips were probed for 1 hour with biotinylated horse anti-mouse IgG (1:200) (Vector Laboratories, BA-2000) and fluorescein-conjugated donkey anti-rabbit IgG (1:50) (Jackson ImmunoResearch, 711-095-152) diluted in 0.2% BSA/PBS. Unbound secondary antibodies were removed with PBS washes, and final dystrophin labeling was done with a 10-minute incubation of rhodamine avidin DCS (1:60) (Vector Laboratories) diluted in PBS.

Human iPSC-cardiomyocyte calcium imaging. Calcium imaging of human iPSC-derived cardiomyocytes was performed as previously described (Atmanli et al., 2019). Beating cardiomyocytes were dissociated into a single-cell suspension and seeded on a glass-bottom dish at single-cell density. Cells were loaded with the fluorescent calcium indicator Fluo-4 AM (Thermo Fisher) at 2 μM for 30 minutes and then cultured in medium containing 0 or 10 μM isoproterenol (Sigma-Aldrich) for another 30 minutes before imaging. Spontaneous Ca2⁺ transients of beating iPSC-derived cardiomyocytes were imaged at 37° C. using a Nikon A1R+ confocal system. Ca2⁺ transients were processed using Fiji software (Schneier et al., 2012) and analyzed using Microsoft Excel.

Statistics. All data are presented as mean±S.E.M. Unpaired two-tailed Student's t tests were performed for comparison between the respective two groups as indicated in the figures. Data analyses were performed with statistical software (GraphPad Prism Software). P values less than 0.05 were considered statistically significant.

### Example 2—Development of ‘Single-Swap’ ABE as an In Vivo Nucleotide Editing Strategy

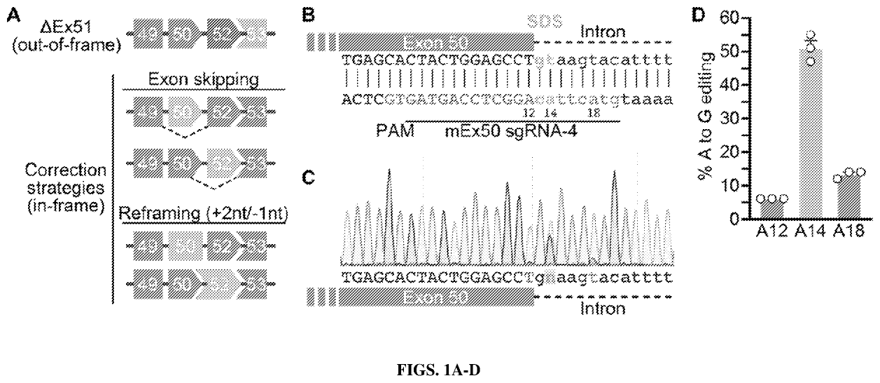

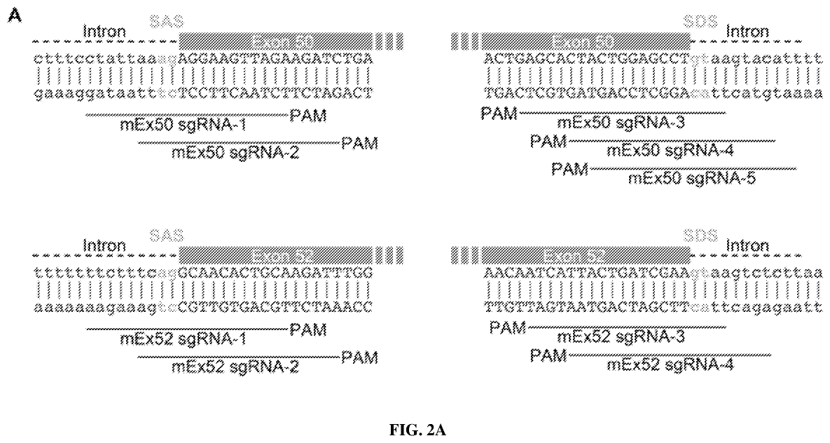

Previously, a DMD mouse model with deletion of exon 51 in the mouse Dmd gene (ΔEx51) was generated and validated (Chemello et al., 2020). These mice display the hallmarks of DMD, including absence of dystrophin, replacement of degenerative muscle fibers with inflammatory cells and fibrotic and fatty tissue, and an increased percentage of centralized nuclei in myofibers, indicative of active myofiber degeneration and regeneration. Deletion of exon 51 in the Dmd gene results in the introduction of a downstream premature stop codon in exon 52 and production of a non-functional truncated dystrophin protein (FIG. 1A, upper panel). The Dmd ORF can be restored by skipping of either exon 50 or exon 52. This allows splicing of exon 49 to exon 52 in the case of exon 50 skipping, or splicing of exon 50 to exon 53 in the case of exon 52 skipping (FIG. 1A, Exon skipping). Another correction strategy involves ‘reframing’ of exon 50 or exon 52 by targeted nucleotide base pair insertions or deletions. Accordingly, insertions of 3n+2 nucleotides or deletions of 3n−1 nucleotides within exon 50 or exon 52 can restore the ORF (FIG. 1A, Reframing). However, these insertions and deletions must occur at sites in the exon that do not result in introduction of a new premature stop codon following the targeted insertions or deletions. Both correction strategies can restore the ORF and lead to production of a functional but internally truncated dystrophin protein.

To restore dystrophin expression in the ΔEx51 mouse model, exon skipping was induced by destroying the SAS or SDS of exon 50 or exon 52 by a ‘Single-Swap’ base pair transition using base editing. The canonical SAS consensus sequence is AG and the canonical SDS consensus sequence is GT, and the pairing of the SAS and SDS define an exon for recognition by the splicing machinery (Berget, 1995). ABEs can disrupt either the SAS or SDS consensus sequences by causing a ‘Single-Swap’ of one of the base pairs in the dinucleotide splicing motifs. Destruction of either splice site bracketing exon 50 or exon 52 could prevent pairing of the splice sites across that exon by the splicing machinery. This would preclude the splicing machinery from recognizing that exon, thereby causing skipping of that exon and restoring the correct ORF of the Dmd transcript.

For exon skipping in ΔEx51 mice, the ABEmax adenine base editor as used, as ABEs produce less off-target editing than CBEs (Grunewald et al., 2019; Jin et al., 2019; Zuo et al., 2019; Lee et al., 2020). ABEmax can edit the adenine in the sense strand of the SAS AG consensus sequence or the adenine in the antisense strand of the SDS GT consensus sequence. Candidate sgRNAs around the SAS and SDS were identified for both exon 50 and exon 52 that had PAMs with an NGG PAM sequence for editing with ABEmax-SpCas9 or the more relaxed NG PAM sequence for editing with the engineered ABEmax-SpCas9-NG (Huang et al., 2019). These sgRNAs also positioned the target SAS or SDS within the canonical base editing window of ABEmax (approximatively nucleotide positions 12-18; counting the PAM nucleotides as −2 to 0 for NGG or −1 to 0 for NG).

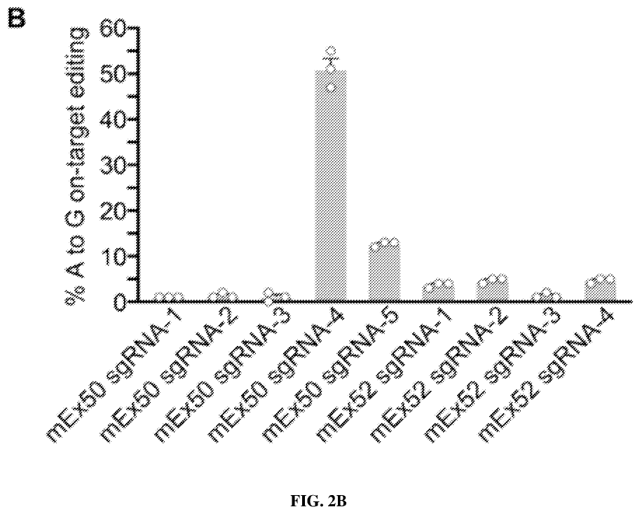

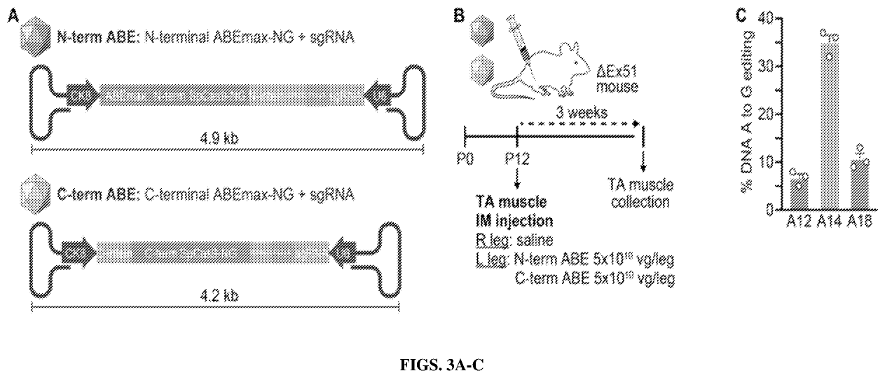

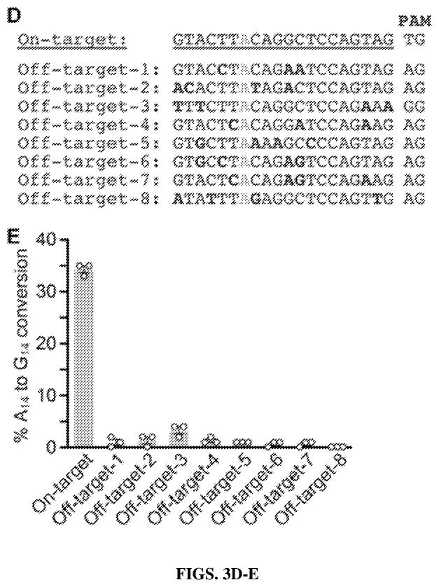

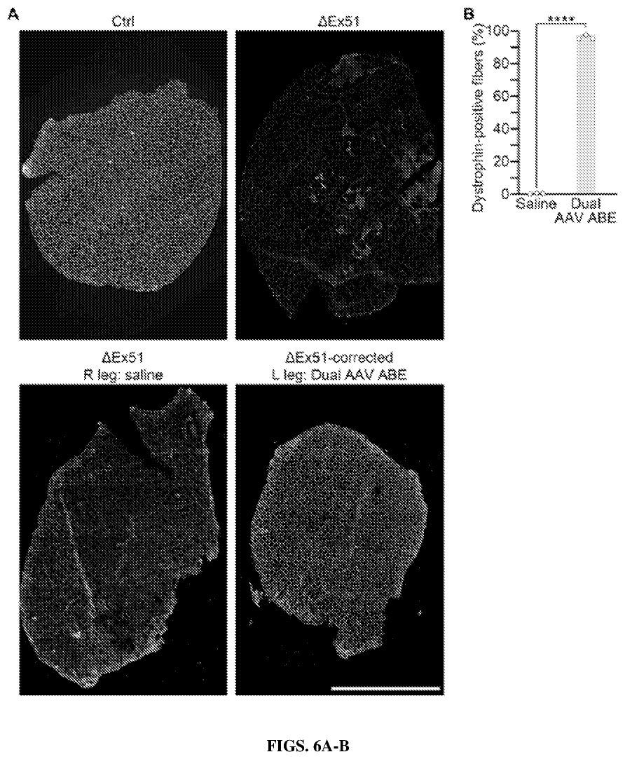

A total of nine candidate sgRNAs were tested in mouse N2a neuroblastoma cells targeting either the SAS or SDS of exon 50 or exon 52 (FIG. 2A). Mouse exon 50 (mEx50) sgRNA-4 together with ABEmax-SpCas9-NG was the most efficient sgRNA overall (FIGS. 1B and 6B). mEx50 sgRNA-4 induced on-target editing of position A14 of 51.0±2.3% in the SDS of exon 50, with minor bystander editing at A12 and A18 (FIGS. 1C and 1D). The other candidate sgRNAs showed low editing efficiency at on-target sites (FIG. 2B). As such, mEx50 sgRNA-4 with ABEmax-SpCas9-NG was selected for further in vivo base editing studies.

### Example 3—AAV Packaging of ABE Components in a Split-Intein System for In Vivo Delivery

Current viral-based gene editing therapies use AAVs which have a packaging limit of <5 kb, which precludes packaging of the ABEmax-SpCas9-NG base editor (˜5.8 kb) into a single AAV vector. Recently, dual AAVs have been described for the delivery of split base editors using trans-splicing inteins, which act as protein introns to enable covalent splicing of N- and C-terminal peptides (Levy et al., 2020). Each half of the split base editor when linked to trans-splicing inteins can reassemble after translation into a functional base editor that retains similar editing efficiencies compared to its non-split, full-length equivalent.

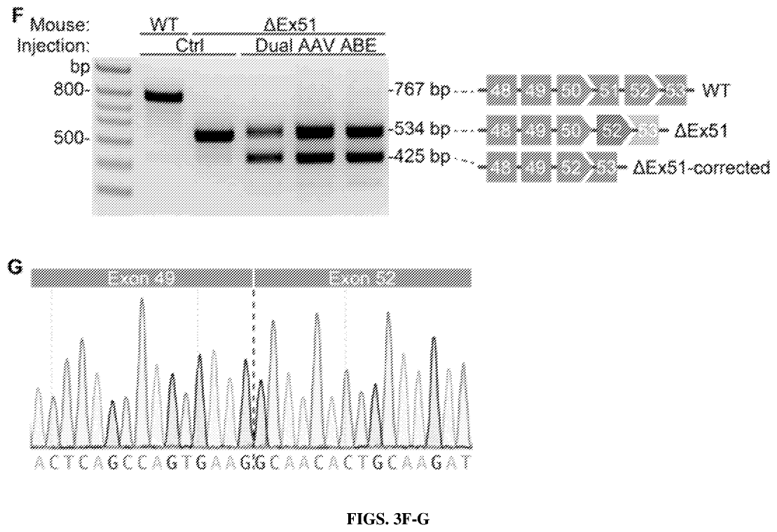

The split-intein system was adapted by dividing ABEmax-SpCas9-NG into two smaller fragments that can each be packaged within separate AAV vectors (FIG. 3A). The N-terminal AAV construct consisted of the N-terminal half of ABEmax-SpCas9-NG fused to one split DnaE intein half from Nostoc punctiforme (Npu) that was expressed under the control of the creatine kinase 8 (CK8e) promoter. This promoter drives high level expression specifically in skeletal muscle and heart (Martari et al., 2009). The C-terminal AAV construct consisted of the other DnaE intein half from Npu fused to the C-terminal half of ABEmax-SpCas9-NG, also driven by the CK8e promoter. Each AAV construct also contained a truncated Woodchuck hepatitis virus post-transcriptional regulatory element (WPRE3) (Choi et al., 2014), two codon-optimized nuclear localization signals each flanking the ABEmax-SpCas9-NG halves, and a U6-driven sgRNA in the reverse orientation. Dual AAV9 particles were then generated encoding each of the terminal halves and mEx50 sgRNA-4 (FIG. 3A).

### Example 4—‘Single-Swap’ ABE in ΔEx51 Mice by AAV9 Delivery Restores Dystrophin Production

To validate the efficacy of the ‘Single-Swap’ gene editing strategy in the ΔEx51 mouse model using ABEmax-SpCas9-NG and mEx50 sgRNA-4, localized intramuscular (IM) injection of the dual AAV9 split-intein system in the left tibialis anterior (TA) muscle (5×1010 vg/leg for each virus) of postnatal day 12 (P12) mice was performed. The right TA muscle was injected with saline solution as a control. Three weeks after IM injection, TA muscles were collected for analyses (FIG. 3B).

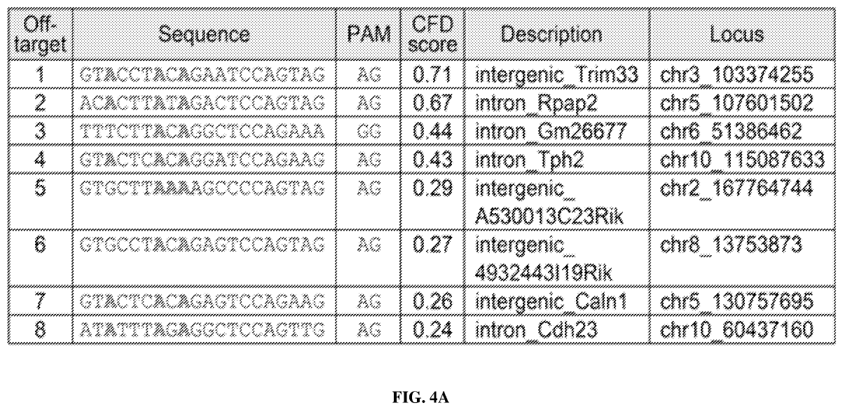

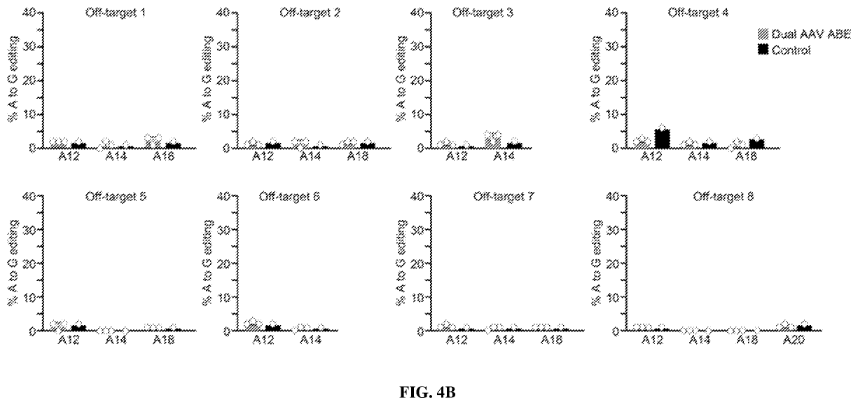

Sequencing of genomic DNA in the treated left leg showed an efficiency of on-target editing of A14 of 35.0±1.5%, with bystander editing at A12 of 6.7±0.9% and A18 of 10.7±1.2% (FIG. 3C). To assess the specificity of base editing, potential off-target editing of the top eight sites predicted by CRISPOR was screened for (Concordet & Haeussler, 2018) (FIG. 3D). Sequence analysis of the candidate off-target amplicons revealed no significant editing at any of the eight tested off-target sites (FIGS. 3E, 4A and 4B).

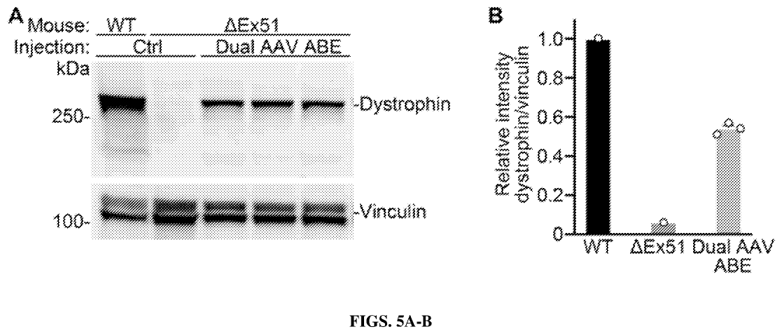

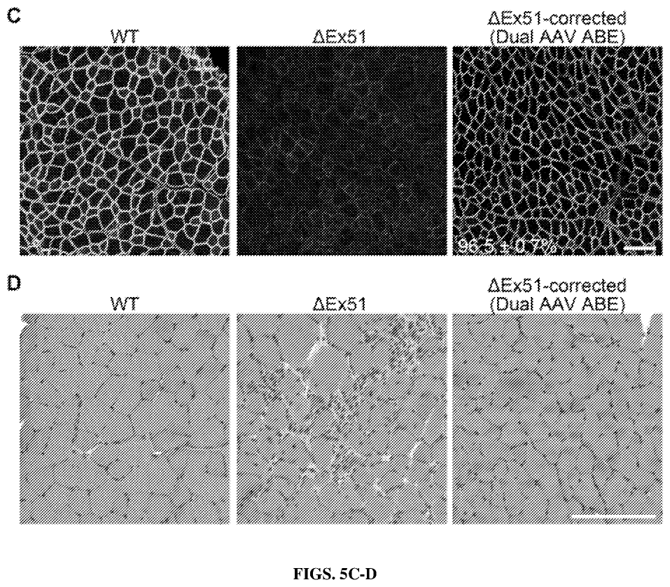

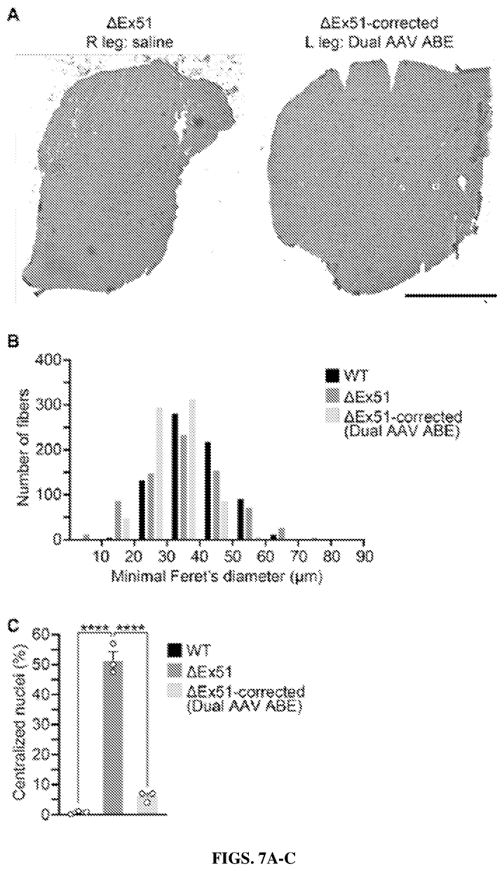

Sequencing of the RT-PCR products generated by primers that amplified the region from exon 48 to exon 53 from the injected TA muscle revealed skipping of exon 50 in mature Dmd mRNA (FIG. 3F, lower band). Skipping of exon 50 allows exon 49 to splice to exon 52 and places the downstream Dmd transcript back in frame (FIG. 3G). Accordingly, Western blot analysis showed restoration of dystrophin protein expression in the AAV-injected TA muscle to a level of 54.0±1.7% compared to WT littermate controls (FIGS. 5A and 5B). Immunohistochemistry showed dystrophin expression was restored in 96.5±0.7% of myofibers (FIGS. 5C, 6A and 6B). Histological analysis by hematoxylin and eosin (H&E) staining showed significant reduction in fibrosis, necrotic myofibers, and regenerating fibers with a reduction in centralized nuclei from 51.5±2.8% to 5.9±1.1%, demonstrating amelioration of the DMD phenotype (FIGS. 5D and 7A-C). Collectively, these data demonstrate that exon skipping mediated by base editing can restore dystrophin expression in developing P12 ΔEx51 mouse TA muscles following IM injection of AAV9 at a dose similar to previously published studies (Amoasii et al., 2017; Min et al., 2019a; Min et al., 2020; Rye et al., 2018; Hakin et al., 2018; Nelson et al., 2019).

### Example 5—a ‘Single-Swap’ ABE Transition Induces Exon Skipping and Restores Dystrophin Expression in Human Cardiomyocytes

Correction of the DMD exon 51 deletion mutation by skipping of exon 50 or exon 52 can therapeutically benefit ˜8% of DMD patients (Aartsma-Rus et al., 2009). To test whether the ‘Single-Swap’ gene editing strategy that was validated in mice was therapeutically translatable to human iPSC-derived cardiomyocytes, exon 51-deleted human iPSCs were generated. Starting with an iPSC line generated from a healthy male donor, CRISPR-Cas9 genomic editing was used to generate an isogenic disease-specific human iPSC line (ΔEx51 iPSCs) with a deletion of exon 51 in the DMD gene. This isogenic pair lessens the possibility of potential intrinsic variations between individual iPSC lines that could lead to misinterpretation of disease-relevant phenotypes (Bock et al., 2011; Boulting et al., 2011).

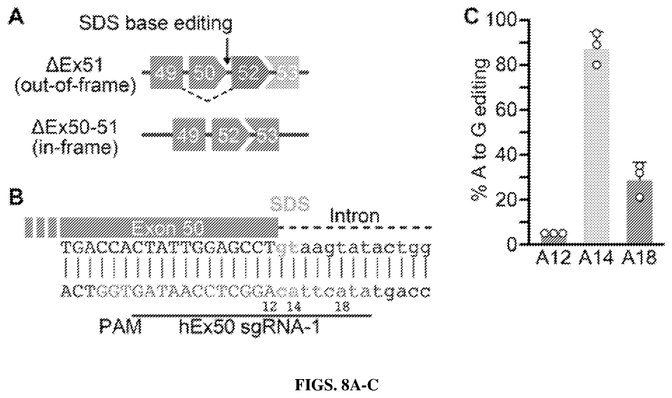

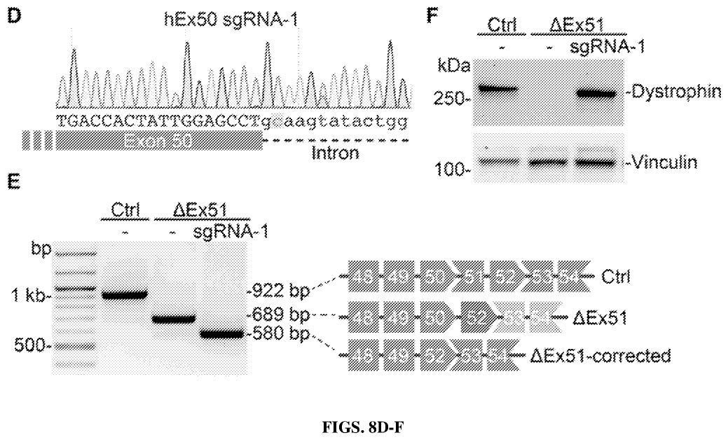

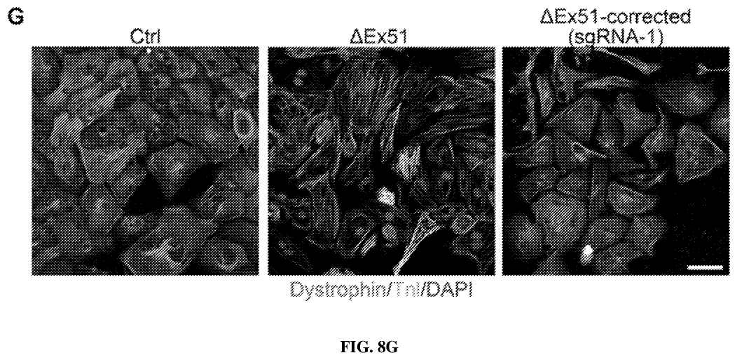

To evaluate the efficiency of base editing of splice sites within the DMD gene by the ABEmax base editor, available sgRNAs with NGG PAMs were screened for editing of the SDS or SAS of exon 50 or exon 52 in human 293T cells. One sgRNA was identified for the SDS of human exon 50 (hEx50 sgRNA-1, Table 3), which has high homology to mEx50 sgRNA-4 used for the previous mouse in vivo experiments, and two sgRNAs for the SAS of human exon 52 (hEx52 sgRNA-2 and −3, Table 3) that positioned the SAS or SDS within the editing window of ABEmax (FIGS. 8A-B and 9A).

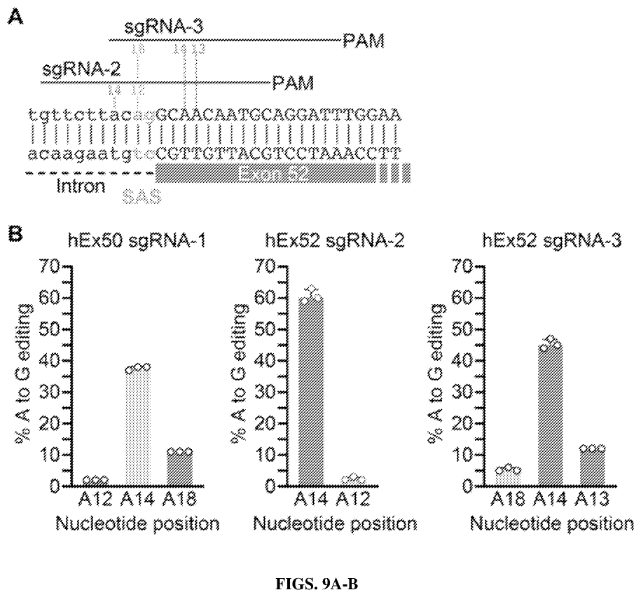

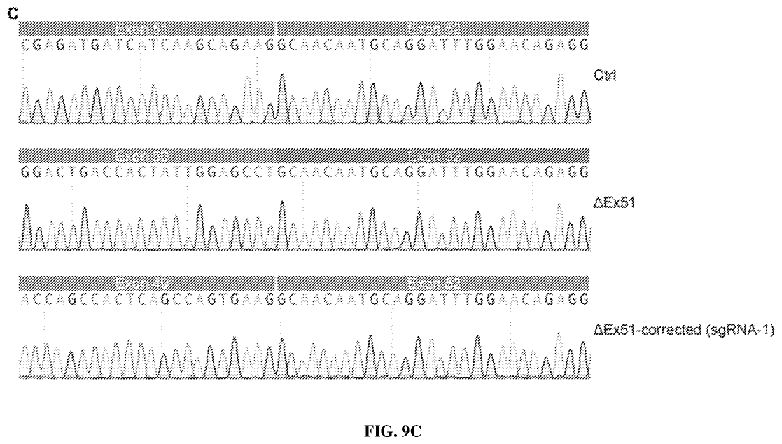

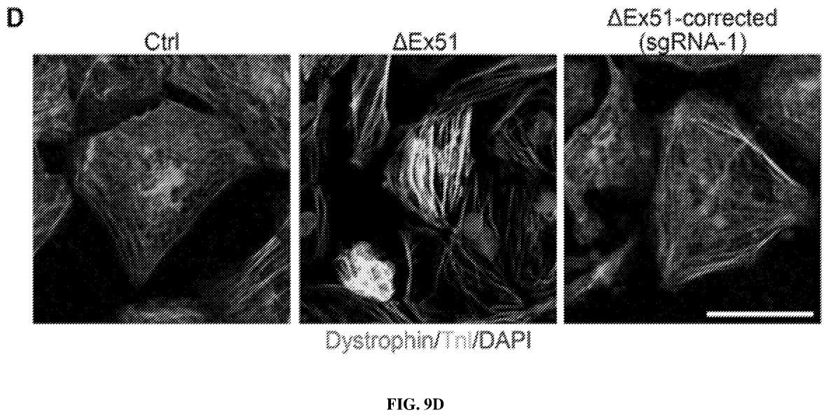

In human 293T cells, hEx50 sgRNA-1 paired with ABEmax-SpCas9 was the most efficient combination of ABE components, with on-target editing of the A:T to G:C base pair in the SDS GT sequence of 38±0.6% (nucleotide position A14) and bystander edits of 2.0±0.0% and 11±0.0% at nucleotide positions A12 and A18, respectively (FIG. 9B). The other two candidate guides, hEx52 sgRNA-2 and -3, paired with ABEmax-SpCas9 were both relatively inefficient at the target A:T base pair (nucleotide position A12 editing of 2.3±0.6% and nucleotide position A18 editing of 5.3±0.6%, respectively) at the SAS AG sequence (FIG. 9B).

Because of its high efficiency in inducing a single nucleotide transition at the SDS of exon 50, hEx50 sgRNA-1 was tested for its ability to promote exon skipping and restore dystrophin expression in human ΔEx51 iPSC-derived cardiomyocytes. Editing in ΔEx51 iPSCs with hEx50 sgRNA-1 and ABEmax-SpCas9 generated on-target editing at A14 of 87.7±4.1%, with bystander editing at A18 of 29.3±4.3% and at A12 of 5.0±0.0% (FIGS. 8C-D). As the bystander edits are located in the intron region or in the to-be-skipped exon, they are not predicted to affect the final dystrophin transcript.

Single clones of iPSCs containing a base edited SDS GT dinucleotide sequence to a GC dinucleotide sequence were isolated and differentiated into cardiomyocytes. RT-PCR using primers that amplify the region from exon 48 to exon 54 and cDNA sequencing analysis showed skipping of exon 50 and splicing of exon 49 to exon 52 (FIGS. 8E and 9C).

Western blot analysis and immunocytochemistry showed restoration of dystrophin protein expression in ΔEx51 cardiomyocytes that had been corrected with hEx50 sgRNA-1 and ABEmax-SpCas9 (FIGS. 8F-G and 9D). These findings are consistent with previous studies on exon skipping correction strategies around the exon 50-51 locus in DMD patient-derived iPSC cardiomyocytes (Long et al., 2018; Dick et al., 2013). Taken together, these data suggests that a single-swap transition generated by ABEmax at the SDS GT sequence of DMD exon 50 is sufficient to cause skipping of exon 50 in human DMD cells and restore dystrophin protein expression.

### Example 6—Prime Editing of DMD Exons can Enable Exon Reframing and Restore Dystrophin Expression in Human Cardiomyocytes

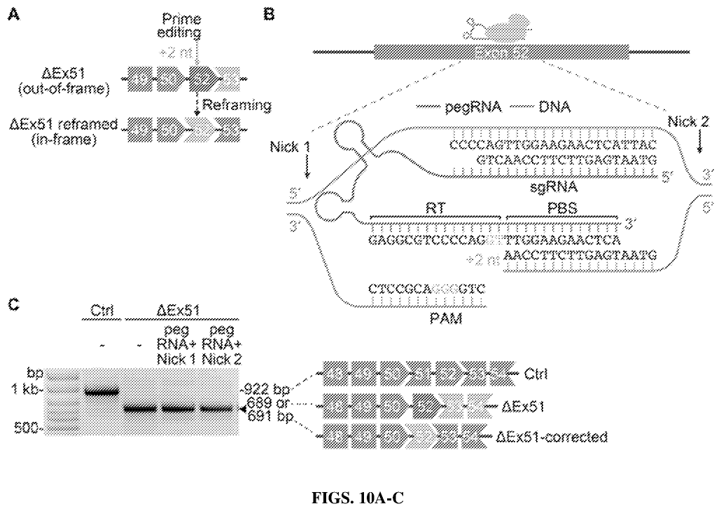

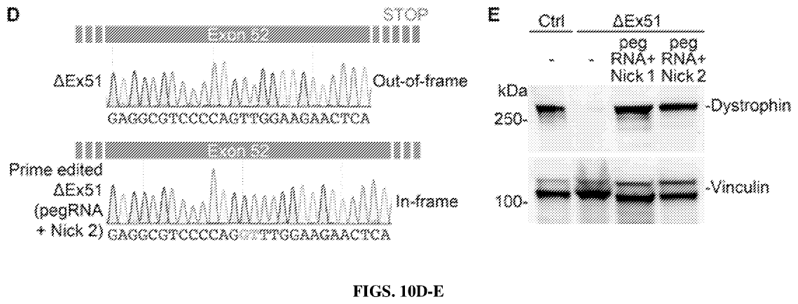

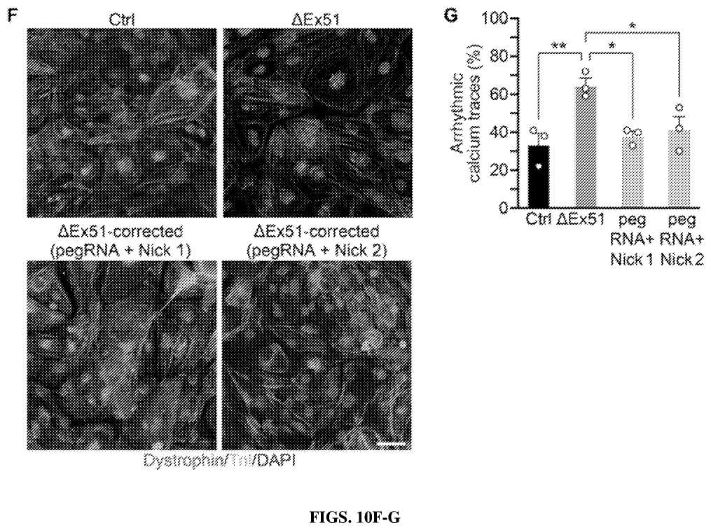

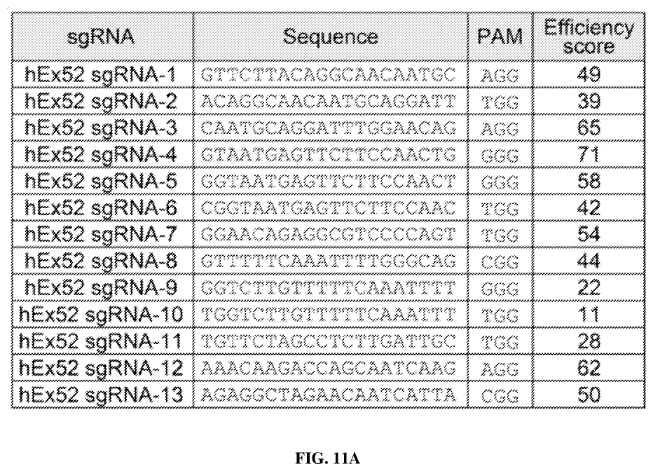

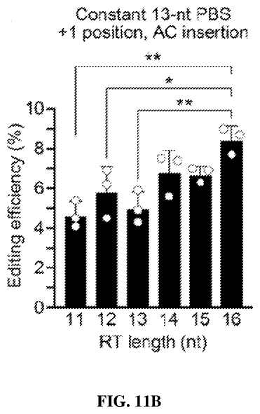

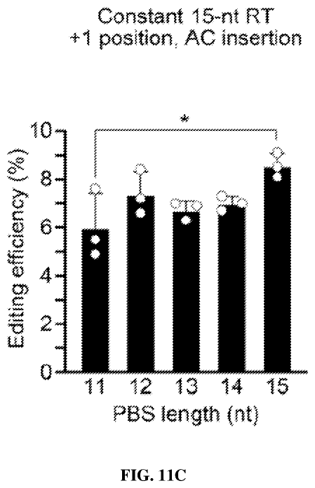

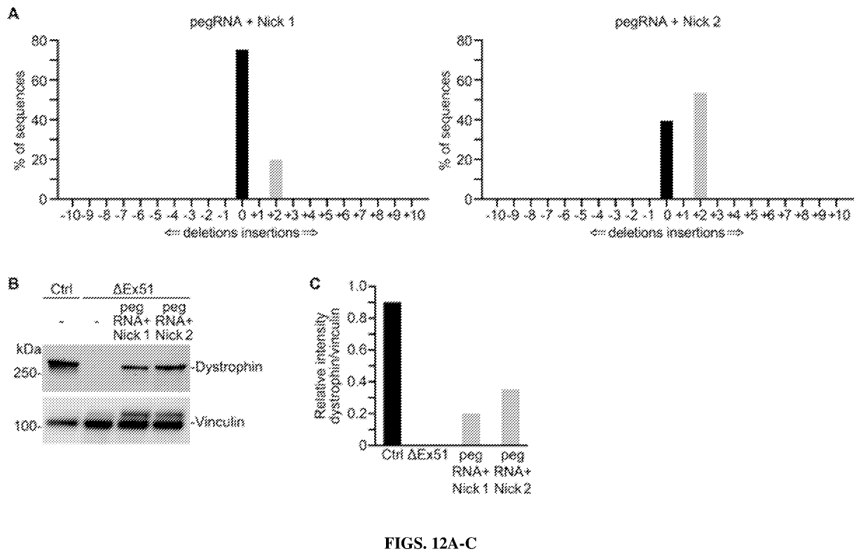

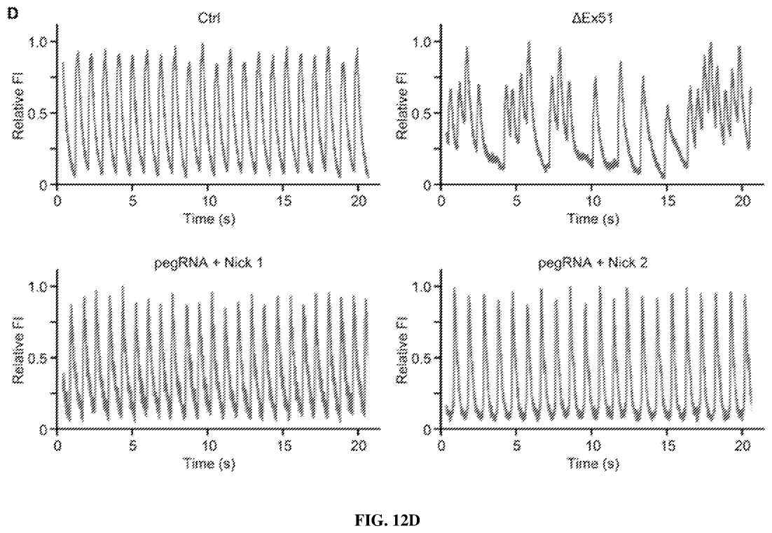

As base editing for exon 52 skipping was found to be relatively inefficient, a prime editing-based reframing strategy was developed for exon 52 as another potential gene editing correction strategy for the exon 51 deletion mutation in iPSC-derived cardiomyocytes (FIG. 10A). It was reasoned that one of the keys to efficient prime editing is the efficiency of the sgRNA in the pegRNA construct. sgRNA efficiency prediction software using CRISPOR suggested that hEx52 sgRNA-4 (Table 4, hEx52g4.PE.spacer) was likely to be the most efficient sgRNA in exon 52 as calculated by scoring from Doench et al. (2014), so this sgRNA was selected for further optimization in the pegRNA construct (FIG. 11A). As prime editing allows discretionary gene insertions and deletions, it was arbitrarily chosen to introduce a +2 nucleotide AC insertion at position +1 with respect to the nicking site generated by hEx52 sgRNA-4 (counting the PAM positions as +4 to +6). As hEx52 sgRNA-4 is in the antisense orientation and inserts the AC dinucleotide sequence on the antisense strand, the final DMD transcript will contain a GT dinucleotide insertion on the sense strand upon successful prime editing (FIG. 10B). Following recommendations for prime editing optimization (Anzalone et al., 2019), a pegRNA with a PBS length of 13 nucleotides and a RT template length of 15 nucleotides (referred to as hEx52-PE) was used as a starting point (FIG. 10B). The lengths of the PBS and RT template were then systematically varied to find the most highly efficient pegRNA (Table 4). While longer lengths of the PBS and RT template correlated with increased editing efficiency, the longest lengths performed comparably (FIGS. 11B-C). To further optimize the editing efficiency, two nicking sgRNAs were selected to pair with hEx52-PE (Table 4), which cause a nick 29 nucleotides upstream (nick-1, −29 nt) or a nick 52 nucleotides downstream on the sense strand (nick-2, +52 nt) with respect to the nicking site generated by hEx52 sgRNA-4 (FIG. 5B).

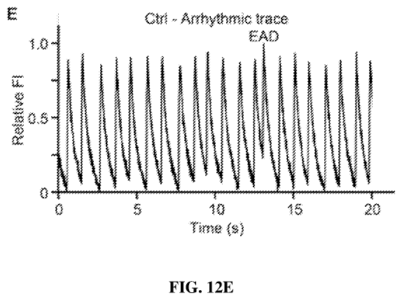

The efficiency of hEx52-PE was tested in the ΔEx51 iPSC model with both nicking sgRNAs. A 20.2% efficiency was detected for introducing a +2 nucleotide GT insertion on the sense strand at the desired position using hEx52-PE and nick-1, and a 54.0% efficiency using hEx52-PE and nick-2 (FIG. 12A). Then, the total mixture of edited and non-edited iPSCs were differentiated into cardiomyocytes to determine the effects of the insertion on dystrophin recovery. The relative quantity of dystrophin protein with respect to the healthy control iPSC-derived cardiomyocytes was 24.8% after editing with hEx52-PE and nick-1, and 39.7% after editing with hEx52-PE and nick-2, which correlated with the DNA editing efficiencies (FIGS. 12B-C).

Single clones of iPSCs with the prime edited insertion in exon 52 were isolated and differentiated into cardiomyocytes. RT-PCR and cDNA sequencing analyses confirmed the precise GT insertion on the sense strand in exon 52 (FIGS. 10C-D). The correct reframing of the ORF was confirmed by the restoration of dystrophin protein expression, as demonstrated by Western blot analysis and immunocytochemistry (FIGS. 10E-F).

### Example 7—Prime Editing of DMD Exons Normalizes Contractile Abnormalities of Human DMD Cardiomyocytes

It was next investigated if genome editing of cardiomyocytes by prime editing could rescue a possible arrhythmic defect in ΔEx51 cardiomyocytes. Thirty-day-old iPSC-derived cardiomyocytes were treated with isoproterenol, and calcium-cycling analyses were performed. An arrhythmic defect was detected in the ΔEx51 cardiomyocytes compared to the healthy control cardiomyocytes. This observation recapitulates patient phenotypes (McNally et al., 2015) and human cardiac iPSC models of DMD (Kyrychenko et al., 2017; Kamdar et al., 2020), with a significant increase in the percentage of arrhythmic calcium traces from 33.7±5.6% of the healthy control cardiomyocytes to 64.7±3.8% of ΔEx51 cardiomyocytes (FIGS. 10G and 12D-E). The observation that a fraction of ΔEx51 cardiomyocytes did not exhibit an arrhythmic phenotype could stem from the transcriptional, structural and functional heterogeneity of iPSC-derived cardiomyocytes (Atmanli et al., 2019; Chirikian et al., 2021), whereby cardiomyocytes from different lineages (i.e. nodal, atrial, ventricular) could display variable susceptibilities to arrhythmias. A similar observation was reported in other studies investigating the electrophysiological properties of iPSC-derived cardiomyocytes in the setting of diseases that affect cardiac electrophysiology (Kamdar et al., 2020; Han et al., 2014; Kuroda et al., 2017). In contrast, prime edited-ΔEx51 cardiomyocytes exhibited a percentage of arrhythmic calcium traces comparable to that of the healthy control cardiomyocytes (38.0±2.5% after editing with hEx52-PE and nick-1, and 41.7±6.6% after editing with hEx52-PE and nick-2), confirming alleviation of the arrhythmic defect in prime edited-reframed ΔEx51 cardiomyocytes (FIGS. 10G and 12D). Taken together, these data demonstrate that prime editing can be used to precisely reframe the correct ORF and restore functional dystrophin expression in cultured human ΔEx51 iPSC-cardiomyocytes when cells are nucleofected and sorted to isolate transfected cells.

### Example 8—Adenine Base Editing of Splice Acceptor Site of DMD Exon 51 Restores Dystrophin Expression in Human ΔEx48-50 DMD Cardiomyocytes

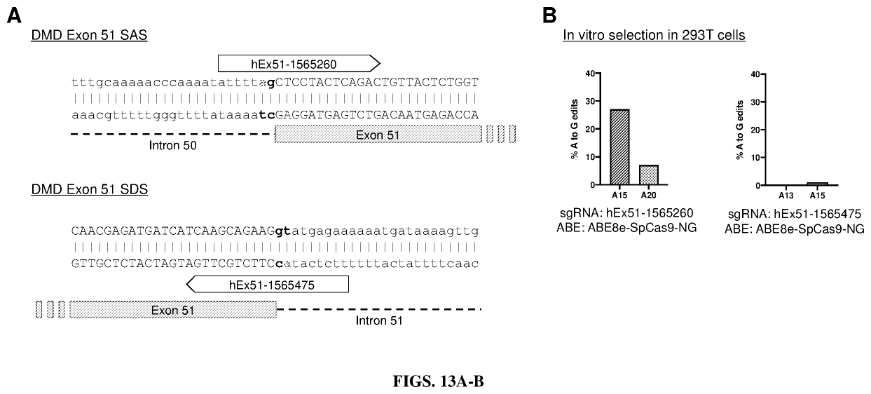

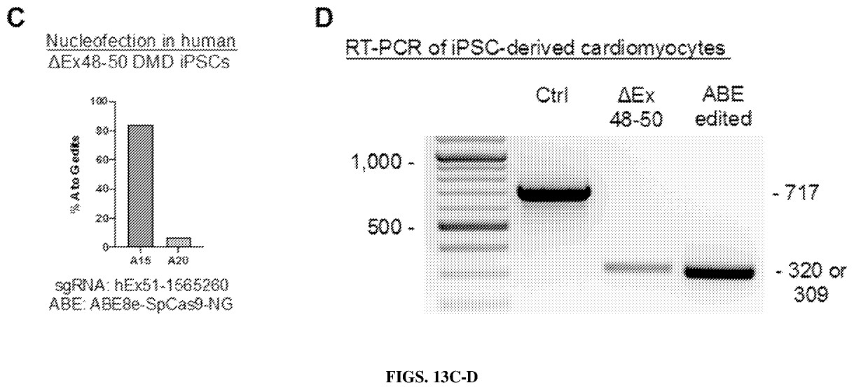

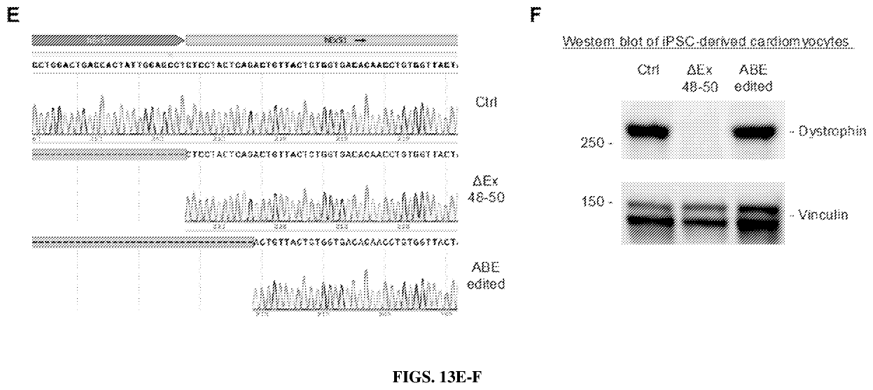

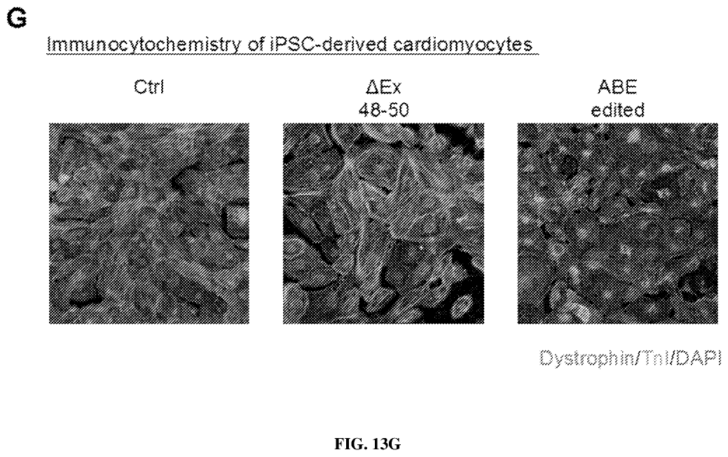

To correctly reframe the DMD transcript in DMD human cells lacking exons 48 to 50 in the DMD gene adenine base editing was applied in the splice sites of exon 51. Two sgRNAs were identified that have an editing window in the desired adenines, and that can be used with the SpCas9-NG (FIG. 13A). For this set of experiments, adenine base editor ABE8e, an improved version of ABE, was used. In vitro experiments in 293T cells showed an editing efficiency of ˜30% for the sgRNA hEx51-1565260 (FIG. 13B) (Table 3). This sgRNA with ABE8e-SpCas9-NG was nucleofected in ΔEx48-50 DMD iPSCs. The editing efficiency in iPSCs for the adenine in the splice acceptor site was 82% (FIG. 13C). Edited ΔEx48-50 DMD iPSCs were differentiated into cardiomyocytes to prove the reframing of the DMD transcript and the restoration of dystrophin. RT-PCR and Sanger sequencing of the edited band showed the utilization of a new cryptic splice site by the splicing machinery, with the deletion of 11 nucleotides of exon 51 and the restoration of the correct ORF of DMD transcript (FIGS. 13D-E). The restoration of dystrophin protein was proved by Western blot and immunocytochemistry analyses (FIGS. 13F-G).

### Example 9—Adenine Base Editing of Splice Acceptor Site of DMD Exon 45 Restores Dystrophin Expression in Human ΔEx44 DMD Cardiomyocytes

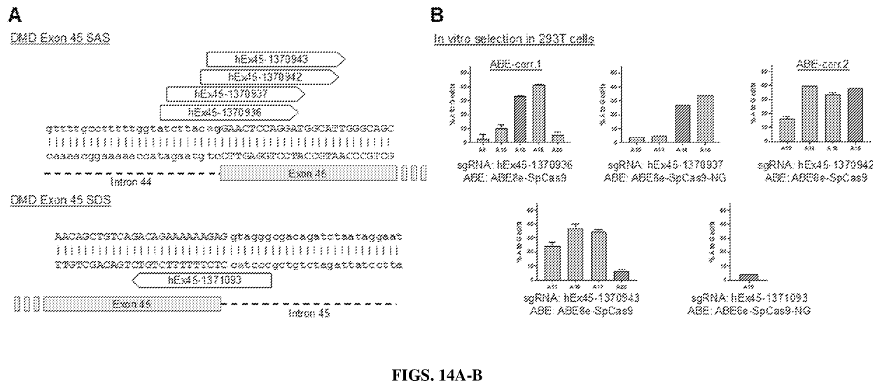

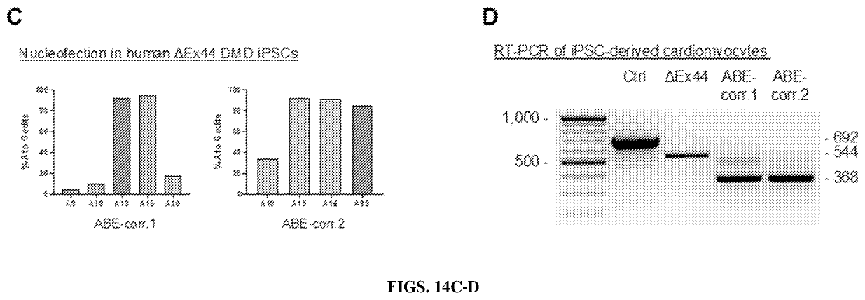

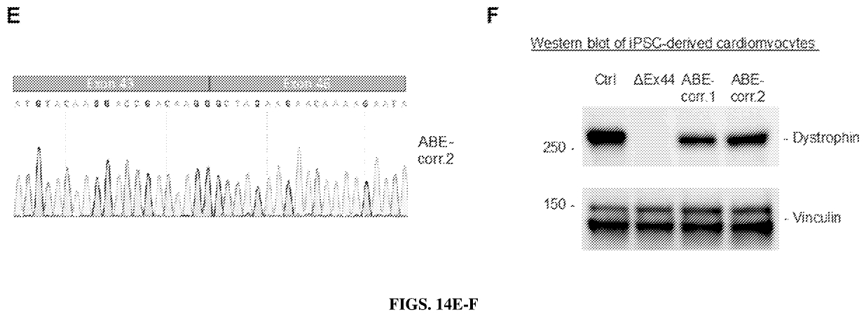

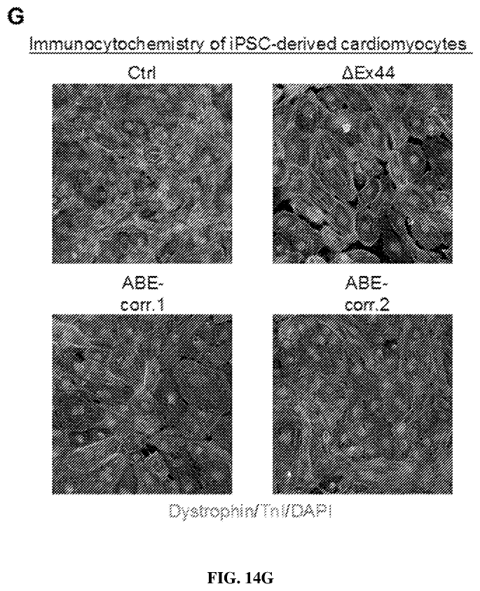

To correctly reframe the DMD transcript in DMD human cells lacking exon 44 in the DMD gene adenine base editing was applied in the splice sites of exon 45. Five sgRNAs were identified that have an editing window in the desired adenines, and that can be used with the Sp-Cas9 or SpCas9-NG (FIG. 14A). For this set of experiments, adenine base editor ABE8e, an improved version of ABE, was used. In vitro experiments in 293T cells showed an editing efficiency of at least 30% for 2 sgRNAs (hEx45-1370936, corr.1; hEx45-1370942, corr.2) (FIG. 14B) (Table 3). These sgRNAs with ABE8e-SpCas9 were nucleofected in ΔEx44 DMD iPSCs. The editing efficiency in iPSCs for the adenine in the splice acceptor site was more than 80% for both the sgRNAs (FIG. 14C). Edited ΔEx44 DMD iPSCs were differentiated into cardiomyocytes to prove the reframing of the DMD transcript and the restoration of dystrophin. RT-PCR and Sanger sequencing of the edited bands showed the splicing of exon 45 and the restoration of the correct ORF of DMD transcript (FIGS. 14D-E). The restoration of dystrophin protein was proved by Western blot and immunocytochemistry analyses (FIGS. 14F-G).

### Example 10—SauriCas9 and SlugCas9 Base Editors Restore Dystrophin Expression in Human DEx44 iPSC-Derived Cardiomyocytes

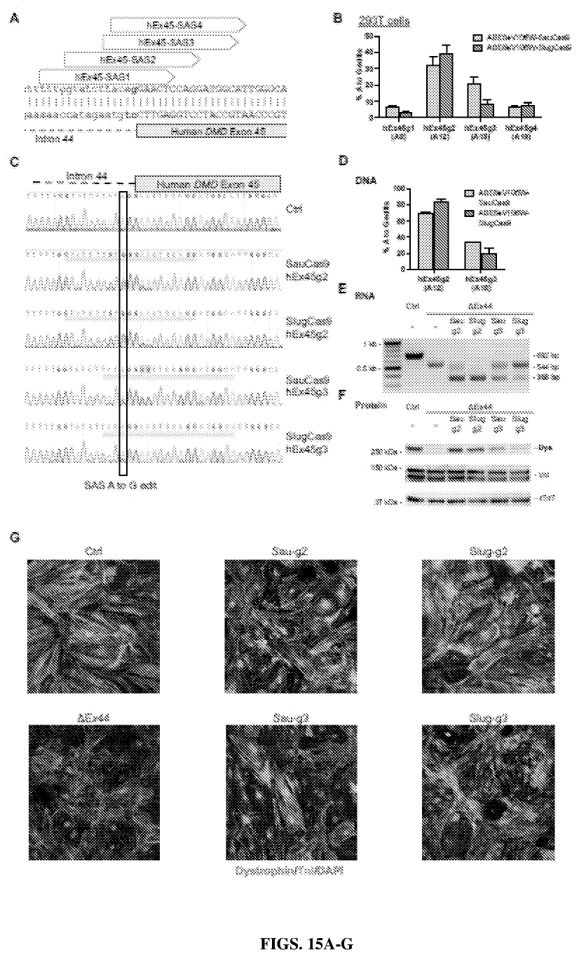

Adenine base editing of splice acceptor site of DMD exon 45 by compact base editors restores dystrophin expression in human ΔEx44 DMD cardiomyocytes. To correctly reframe the DMD transcript in DMD human cells lacking exon 44 in the DMD gene adenine base editing was applied in the splicing acceptor site of exon 45 using compact base editors. ABE8eV106W was fused to SauriCas9 (SauCas9) or SlugCas9 to generate compact base editors. Four sgRNAs were identified that have an editing window in the desired adenine, and that can be used with the ABE8eV106W-SauCas9 or -SlugCas9 (FIG. 15A). In vitro experiments In 293T cells showed high editing efficiency for hEx45g2 and hEx45g3 (respectively hEx45-Sau-1370935 and hEx45-Sa-1370941 in Table 3) (FIG. 15B). These sgRNAs with ABE8eV106W-SauCas9 or -SlugCas9 were nucleofected in ΔEx44 DMD iPSCs, confirming their efficacy in editing the genome in the target site (FIG. 15C and FIG. 15D). Nucleofected ΔEx44 DMD iPSCs were differentiated into cardiomyocytes to prove the reframing of the DMD transcript and the restoration of dystrophin. RT-PCR showed the exon skipping of exon 45 (FIG. 15D). The restoration of dystrophin expression was proven by Western blot and immunocytochemistry analyses (FIG. 15F and FIG. 15G).

### Example 11—Generation of ΔEx51 Human iPSCs Using CRISPR-Cas9-Mediated Genome Editing

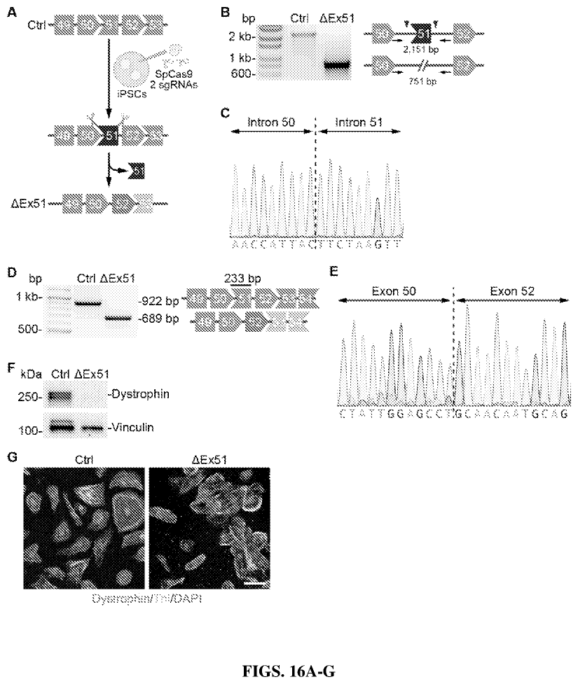

Generation of ΔEx51 human iPSCs using CRISPR-Cas9-mediated genome editing. To generate human induced pluripotent stem cells (hiPSCs) lacking exon 51 of DMD gene (ΔEx51 hiPSCs), healthy control hiPSCs were nucleofected with SpCas9 and two single guide RNAs (sgRNAs) flanking exon 51, in the DMD introns 50 and 51 (FIG. 16A). The 20-nucleotide sequence of the spacer of the sgRNA targeting DMD intron 50 is: TGCATCTTAACCATTACCAT (SEQ ID NO: 173). The 20-nucleotide sequence of the spacer of the sgRNA targeting DMD intron 51 is: GCACAGACAACTTAGAAGAG (SEQ ID NO: 174). This results in the deletion of a 1,400 nucleotides DMD genomic region containing DMD exon 51 (FIG. 16B) and the formation of a new junction between DMD intron 50 and intron 51 (FIG. 16C). In ΔEx51 hiPSC-derived cardiomyocytes, reverse transcriptase polymerase chain reaction (RT-PCR) detected the deletion of exon 51 in the DMD messenger RNA (mRNA) (FIG. 16D), with the consequent splicing of DMD mRNA exon 50 to exon 52 (FIG. 16E). This leads to the generation of a premature stop codon in DMD exon 52 and to the absence of dystrophin protein in ΔEx51 hiPSC-derived cardiomyocytes (FIGS. 16F-G).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Aartsma-Ru et al., Theoretic applicability of antisense-mediated exon skipping for Duchenne muscular dystrophy mutations. Hum Mutat 30, 293-299 (2009).
Aida et al., Prime editing primarily induces undesired outcomes in mice. bioRxiv preprint, (2020).
Amoasii et al., Single-cut genome editing restores dystrophin expression in a new mouse model of muscular dystrophy. Sci Transl Med 9, eaan8081 (2017).
Amoasii et al., Gene editing restores dystrophin expression in a canine model of Duchenne muscular dystrophy. Science 362, 86-91 (2018).
Anzalone et al., Search-and-replace genome editing without double-strand breaks or donor DNA. Nature 576, 149-157 (2019).
Atmanli et al., Multiplex live single-cell transcriptional analysis demarcates cellular functional heterogeneity. Elife 8, e49599 (2019).
Bengtsson et al., Muscle-specific CRISPR/Cas9 dystrophin gene editing ameliorates pathophysiology in a mouse model for Duchenne muscular dystrophy. Nat Commun 8, 14454 (2017).
Berget, Exon recognition in vertebrate splicing. J Biol Chem 270, 2411-2414 (1995).
Bladen et al., The TREAT-NMD DMD Global Database: analysis of more than 7,000 Duchenne muscular dystrophy mutations. Hum Mutat 36, 395-402 (2015).
Bock et al., Reference Maps of human ES and iPS cell variation enable high-throughput characterization of pluripotent cell lines. Cell 144, 439-452 (2011).
Boulting et al., A functionally characterized test set of human induced pluripotent stem cells. Nat Biotechnol 29, 279-286 (2011).
Briguet et al., Histological parameters for the quantitative assessment of muscular dystrophy in the mdx-mouse. Neuromuscul Disord 14, 675-682 (2004).
Brinkman et al., Easy quantitative assessment of genome editing by sequence trace decomposition. Nucleic Acids Res 42, (2014).
Burridge et al., Chemically defined generation of human cardiomyocytes. Nat Methods 11, 855-860 (2014).
Chakrabarti et al., Target-Specific Precision of CRISPR-Mediated Genome Editing. Mol Cell 73, 699-713 e696 (2019).
Chemello et al., Degenerative and regenerative pathways underlying Duchenne muscular dystrophy revealed by single-nucleus RNA sequencing. Proc Natl Acad Sci USA 117, 29691-29701 (2020a).
Chemello et al., Correction of muscular dystrophies by CRISPR gene editing. J Clin Invest 130, 2766-2776 (2020b).
Chirikian et al., CRISPR/Cas9-based targeting of fluorescent reporters to human iPSCs to isolate atrial and ventricular-specific cardiomyocytes. Sci Rep 11, 3026 (2021).
Choi et al., Optimization of AAV expression cassettes to improve packaging capacity and transgene expression in neurons. Mol Brain 7, 17 (2014).
Concordet & Haeussler, CRISPOR: intuitive guide selection for CRISPR/Cas9 genome editing experiments and screens. Nucleic Acids Res 46, W242-W245 (2018).
Dick et al., Exon skipping and gene transfer restore dystrophin expression in human induced pluripotent stem cells-cardiomyocytes harboring DMD mutations. Stem Cells Dev 22, 2714-2724 (2013).
Doench et al., Rational design of highly active sgRNAs for CRISPR-Cas9-mediated gene inactivation. Nat Biotechnol 32, 1262-1267 (2014).
Duan et al., Expanding AAV packaging capacity with trans-splicing or overlapping vectors: a quantitative comparison. Mol Ther 4, 383-391 (2001).
Echigoya et al., Multiple exon skipping in the Duchenne muscular dystrophy hot spots: prospects and challenges. J Pers Med 8, 41 (2018).
Flanigan et al., Mutational spectrum of DMD mutations in dystrophinopathy patients: application of modern diagnostic techniques to a large cohort. Hum Mutat 30, 1657-1666 (2009).
Gapinske et al., CRISPR-SKIP: programmable gene splicing with single base editors. Genome Biol 19, 107 (2018).
Goldstein et al., In Situ Modification of Tissue Stem and Progenitor Cell Genomes. Cell Rep 27, 1254-1264 e1257 (2019).
Grunewald et al., Transcriptome-wide off-target RNA editing induced by CRISPR-guided DNA base editors. Nature 569, 433-437 (2019).
Hakim et al., AAV CRISPR editing rescues cardiac and muscle function for 18 months in dystrophic mice. JCI Insight 3, (2018).
Han et al., Study familial hypertrophic cardiomyopathy using patient-specific induced pluripotent stem cells. Cardiovasc Res 104, 258-269 (2014).
Hoffman et al., Dystrophin—the Protein Product of the Duchenne Muscular-Dystrophy Locus. Cell 51, 919-928 (1987).
Huang et al., Circularly permuted and PAM-modified Cas9 variants broaden the targeting scope of base editors. Nat Biotechnol 37, 626-631 (2019).
Jin et al., Cytosine, but not adenine, base editors induce genome-wide off-target mutations in rice. Science 364, 292-295 (2019).
Kamdar et al., Stem cell-derived cardiomyocytes and beta-adrenergic receptor blockade in Duchenne muscular dystrophy cardiomyopathy. J Am Coll Cardiol 75, 1159-1174 (2020).
Kim et al., Highly efficient RNA-guided base editing in mouse embryos. Nat Biotechnol 35, 435-437 (2017).
Kluesner et al., EditR: A Method to Quantify Base Editing from Sanger Sequencing. CRISPR J 1, 239-250 (2018).
Koblan et al., Improving cytidine and adenine base editors by expression optimization and ancestral reconstruction. Nat Biotechnol 36, 843-846 (2018).
Kuroda et al., Flecainide ameliorates arrhythmogenicity through NCX flux in Andersen-Tawil syndrome-iPS cell-derived cardiomyocytes. Biochem Biophys Rep 9, 245-256 (2017).
Kwon et al., In Vivo Gene Editing of Muscle Stem Cells with Adeno-Associated Viral Vectors in a Mouse Model of Duchenne Muscular Dystrophy. Mol Ther Methods Clin Dev 19, 320-329 (2020).
Kyrychenko et al., Functional correction of dystrophin actin binding domain mutations by genome editing. JCI Insight 2, e95918 (2017).
Lee et al., Cytosine base editor 4 but not adenine base editor generates off-target mutations in mouse embryos. Communications Biology 3, 19 (2020).
Levy et al., Cytosine and adenine base editing of the brain, liver, retina, heart and skeletal muscle of mice via adeno-associated viruses. Nat Biomed Eng 4, 97-110 (2020).
Long et al., Postnatal genome editing partially restores dystrophin expression in a mouse model of muscular dystrophy. Science 351, 400-403 (2016).
Long et al., Correction of diverse muscular dystrophy mutations in human engineered heart muscle by single-site genome editing. Sci Adv 4, eaap9004 (2018).
Martari et al., Partial Rescue of Growth Failure in Growth Hormone (GH)-Deficient Mice by a Single Injection of a Double-Stranded Adeno-Associated Viral Vector Expressing the GH Gene Driven by a Muscle-Specific Regulatory Cassette. Human Gene Therapy 20, 759-766 (2009).
McNally et al., Parent Project Muscular, Contemporary cardiac issues in Duchenne muscular dystrophy. Working Group of the National Heart, Lung, and Blood Institute in collaboration with Parent Project Muscular Dystrophy. Circulation 131, 1590-1598 (2015).
Min et al., CRISPR-Cas9 corrects Duchenne muscular dystrophy exon 44 deletion mutations in mice and human cells. Sci Adv 5, eaav4324 (2019a).
Min et al., CRISPR Correction of Duchenne Muscular Dystrophy. Annu Rev Med 70, 239-255 (2019b).
Min et al., Correction of Three Prominent Mutations in Mouse and Human Models of Duchenne Muscular Dystrophy by Single-Cut Genome Editing. Mol Ther 28, 2044-2055 (2020).
Moretti et al., Somatic gene editing ameliorates skeletal and cardiac muscle failure in pig and human models of Duchenne muscular dystrophy. Nature Medicine 26, 207-214 (2020).
Muntoni et al., Dystrophin and mutations: one gene, several proteins, multiple phenotypes. Lancet Neurol 2, 731-740 (2003).
Nelson et al., In vivo genome editing improves muscle function in a mouse model of Duchenne muscular dystrophy. Science 351, 403-407 (2016).
Nelson et al., Long-term evaluation of AAV-CRISPR genome editing for Duchenne muscular dystrophy. Nat Med 25, 427-432 (2019).
Nishimasu et al., Engineered CRISPR-Cas9 nuclease with expanded targeting space. Science 361, 1259-1262 (2018).
Ran et al., Genome engineering using the CRISPR-Cas9 system. Nat Protoc 8, 2281-2308 (2013).
Rees & Liu, Base editing: precision chemistry on the genome and transcriptome of living cells. Nat Rev Genet 19, 770-788 (2018).
Ryu et al., Adenine base editing in mouse embryos and an adult mouse model of Duchenne muscular dystrophy. Nat Biotechnol 36, 536-539 (2018).
Schneider et al., NIH Image to ImageJ: 25 years of image analysis. Nat Methods 9, 671-675 (2012).
Tornabene et al., Intein-mediated protein trans-splicing expands adeno-associated virus transfer capacity in the retina. Sci Transl Med 11, eaav4523 (2019).
Yuan et al., Genetic Modulation of RNA Splicing with a CRISPR-Guided Cytidine Deaminase. Mol Cell 72, 380-394 e387 (2018).
Zuo et al., Cytosine base editor generates substantial off-target single-nucleotide variants in mouse embryos. Science 364, 289-292 (2019).

1. A guide RNA (gRNA) comprising a targeting nucleic acid sequence selected from those disclosed in Table 3.

2. The gRNA of claim 1, wherein the gRNA is a single-molecule guide RNA (sgRNA).

3. The gRNA of claim 1, wherein the gRNA is for modifying a splice site in the human dystrophin gene.

4. A composition comprising a gRNA that targets a splice site of one of exons 45, 50, and 51 of human DMD and a base editor.

5. The composition of claim 4, wherein the base editor is an adenine base editor (ABE).

6. The composition of claim 4, wherein the gRNA is the gRNA of any one of claims 1-3.

7. The composition of claim 6, wherein the base editor is an adenine base editor (ABE).

8. The composition of claim 4, wherein the base editor comprises a CRISPR/Cas nucleases linked to an adenosine deaminase.

9. The composition of claim 8, wherein the CRISPR/Cas nuclease is catalytically impaired.

10. The composition of claim 8, wherein the CRISPR/Cas nuclease is a Cas9 nuclease.

11. The composition of claim 10, wherein the Cas9 nuclease is isolated or derived from Streptococcus pyogenes (spCas9).

12. A nucleic acid comprising:
a sequence encoding a first gRNA of claim 1,
a sequence encoding a base editor,
a sequence encoding a first promoter, wherein the first promoter drives expression of the sequence encoding the first gRNA, and
a sequence encoding a second promoter, wherein the second promoter drives expression of the sequence encoding the base editor.

13. The nucleic acid of claim 12, wherein the base editor is an adenine base editor (ABE).

14. The nucleic acid of claim 12, wherein the base editor comprises a CRISPR/Cas nuclease linked to an adenosine deaminase.

15. The nucleic acid of claim 14, wherein the CRISPR/Cas nuclease is catalytically impaired.

16. The nucleic acid of claim 14, wherein the CRISPR/Cas nuclease is a Cas9 nuclease.

17. The nucleic acid of claim 16, wherein the Cas9 nuclease is isolated or derived from Streptococcus pyogenes (spCas9), Staphylococcus aureus (SaCas9), Staphylococcus auricularis (SauCas9), or Staphylococcus lugdunensis (SlugCas9).

18. The nucleic acid claim 12, wherein at least one of the sequence encoding the first promoter and the sequence encoding the second promoter comprises a cell-type specific promoter.

19. The nucleic acid of claim 18, wherein the cell-type specific promoter is a muscle-specific promoter.

20. The nucleic acid of claim 19, wherein the muscle-specific promoter is a CK8 promoter.

21. The nucleic acid of claim 19, wherein the muscle-specific promoter is a CK8e promoter.

22. The nucleic acid of claim 12, wherein the sequence encoding the first promoter comprises a sequence encoding a U6 promoter, an H1 promoter, or a 7SK promoter.

23. The nucleic acid of claim 12, wherein the nucleic acid comprises a DNA sequence.

24. The nucleic acid of claim 12, wherein the nucleic acid comprises an RNA sequence.

25. The nucleic acid of claim 12, wherein the nucleic acid further comprises a polyadenosine (polyA) sequence.

26. The nucleic acid of claim 25, wherein the polyA sequence is a mini polyA sequence.

27. A cell comprising the nucleic acid of claim 12.

28. A composition comprising the nucleic acid of claim 12.

29. A cell comprising the composition of claim 28.

30. A composition comprising the cell of claim 29.

31. A vector comprising the nucleic acid of claim 12.

32. The vector of claim 31, wherein the vector further comprises a sequence encoding an inverted terminal repeat (ITR) of a transposable element.

33. The vector of claim 32, wherein the transposable element is a transposon.

34. The vector of claim 33, wherein the transposon is a Tn7 transposon.

35. The vector of claim 34, wherein the vector further comprises a sequence encoding a 5′ ITR of a T7 transposon and a sequence encoding a 3′ ITR of a T7 transposon.

36. The vector of claim 12, wherein the vector is a non-viral vector.

37. The vector of claim 36, wherein the non-viral vector is a plasmid.

38. The vector of claim 12, wherein the vector is a viral vector.

39. The vector of claim 38, wherein the viral vector is an adeno-associated viral (AAV) vector or an adenoviral vector.

40. The vector of claim 39, wherein the AAV vector is replication-defective or conditionally replication defective.

41. The vector of claim 39, wherein the AAV vector is a recombinant AAV vector.

42. The vector of claim 39, wherein the AAV vector comprises a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11) or any combination thereof.

43. The vector of claim 39, wherein the AAV vector comprises a sequence isolated or derived from an AAV vector of serotype 9 (AAV9).

44. The vector of claim 39, wherein the AAV vector comprises a sequence isolated or derived from an AAV vector of serotype 2 (AAV2).

45. The vector of claim 39, wherein the AAV vector comprises a sequence isolated or derived from an AAV2 and a sequence isolated or derived from an AAV9.

46. The vector of claim 31, wherein the vector is optimized for expression in mammalian cells.

47. The vector of claim 31, wherein the vector is optimized for expression in human cells.

48. A composition comprising the vector of claim 31.

49. The composition of claim 48, further comprising a pharmaceutically acceptable carrier.

50. A cell comprising the composition of claim 46.

51. The cell of claim 50, wherein the cell is a human cell.

52. The cell of claim 50, wherein the cell is a muscle cell or satellite cell.

53. The cell of claim 50, wherein the cell is an induced pluripotent stem (iPS) cell.

54. A composition comprising the cell of claim 50.

55. A method for correcting a dystrophin defect, the method comprising contacting a cell with a composition of claim 48 under conditions suitable for expression of the first gRNA and the adenine base editor, wherein the first gRNA forms a complex with the adenine base editor, wherein the complex modifies a dystrophin splice site thereby restoring correct open reading frame of DMD transcript.

56. A cell produced by the method of claim 55.

57. A method of treating muscular dystrophy in a subject in need thereof, the method comprising administering to the subject a therapeutically effective amount of a composition of claim 48.

58. The method of claim 57, wherein the composition is administered locally.

59. The method of claim 57, wherein the composition is administered directly to a muscle tissue.

60. The method of claim 57, wherein the composition is administered by an intramuscular infusion or injection.

61. The method of claim 57, wherein the composition is administered systemically.

62. The method of claim 61, wherein the composition is administered by an intravenous infusion or injection.

63. The method of claim 57, wherein, following administration of the composition, the subject exhibits normal dystrophin-positive myofibers, and mosaic dystrophin-positive myofibers containing centralized nuclei, or a combination thereof.

64. The method of claim 57, wherein, following administration of the composition, the subject exhibits an emergence or an increase in a level of abundance of normal dystrophin-positive myofibers when compared to an absence or an level of abundance of normal dystrophin-positive myofibers prior to administration of the composition.

65. The method of claim 57, wherein, following administration of the composition, the subject exhibits an emergence or an increase in a level of abundance of mosaic dystrophin-positive myofibers containing centralized nuclei when compared to an absence or an level of abundance of mosaic dystrophin-positive myofibers containing centralized nuclei prior to administration of the composition.

66. The method of claim 57, wherein, following administration of the composition, the subject exhibits a decreased serum CK level when compared to a serum CK level prior to administration of the composition.

67. The method of claim 55, wherein, following administration of the composition, the subject exhibits improved grip strength when compared to a grip strength prior to administration of the composition.

68. The method of claim 55, wherein the subject is a neonate, an infant, a child, a young adult, or an adult.

69. The method of claim 55, wherein the subject has muscular dystrophy.

70. The method of claim 55, wherein the subject is a genetic carrier for muscular dystrophy.

71. The method of claim 55, wherein the subject is male.

72. The method of claim 55, wherein the subject is female.

73. The method of claim 55, wherein the subject appears to be asymptomatic and wherein a genetic diagnosis reveals a mutation in one or both copies of a DMD gene that impairs function of the DMD gene product.

74. The method of claim 55, wherein the subject presents an early sign or symptom of muscular dystrophy.

75. The method of claim 74, wherein the early sign or symptom of muscular dystrophy comprises loss of muscle mass or proximal muscle weakness.

76. The method of claim 75, wherein the loss of muscle mass or proximal muscle weakness occurs in one or both leg(s) and/or a pelvis, followed by one or more upper body muscle(s).

77. The method of claim 76, wherein the early sign or symptom of muscular dystrophy further comprises pseudohypertrophy, low endurance, difficulty standing, difficulty walking, difficulty ascending a staircase or a combination thereof.

78. The method of claim 55, wherein the subject presents a progressive sign or symptom of muscular dystrophy.

79. The method of claim 78, wherein the progressive sign or symptom of muscular dystrophy comprises muscle tissue wasting, replacement of muscle tissue with fat, or replacement of muscle tissue with fibrotic tissue.

80. The method of claim 55, wherein the subject presents a later sign or symptom of muscular dystrophy.

81. The method of claim 80, wherein the later sign or symptom of muscular dystrophy comprises abnormal bone development, curvature of the spine, loss of movement, and paralysis.

82. The method of claim 55, wherein the subject presents a neurological sign or symptom of muscular dystrophy.

83. The method of claim 82, wherein the neurological sign or symptom of muscular dystrophy comprises intellectual impairment and paralysis.

84. The method of claim 55, wherein the administration of the composition occurs prior to the subject presenting one or more progressive, later or neurological signs or symptoms of muscular dystrophy.

85. The method of claim 55, wherein the subject is less than 10 years old.

86. The method of claim 85, wherein the subject is less than 5 years old.

87. The method of claim 86, wherein the subject is less than 2 years old.

88. (canceled)

89. A guide RNA (gRNA) comprising a targeting nucleic acid sequence selected from those of Table 4.

90. The gRNA of claim 89, comprising a targeting nucleic acid sequence of 5′-GTAATGAGTTCTTCCAACTG-3′ (SEQ ID NO: 1).

91. The gRNA of claim 89, wherein the gRNA is a prime editing (pe) gRNA (pegRNA).

92. The gRNA of claim 89, wherein the gRNA is for modifying the human dystrophin gene to restore the correct open reading frame of a DMD transcript.

93. The gRNA of claim 90, wherein the gRNA further comprises a primer binding site comprising a nucleic acid sequence of 5′-TTGGAAGAACTCA-3′ (SEQ ID NO: 2).

94. The gRNA of claim 93, wherein the gRNA further comprises a reverse transcriptase template comprising a nucleic acid sequence of 5′-GAGGCGTCCCCAGGT-3′ (SEQ ID NO: 3).

95. A composition comprising a gRNA that targets exon 52 of human DMD and a prime editor.

96. The composition of claim 95, wherein the prime editor comprises a CRISPR/Cas nuclease linked to a reverse transcriptase.

97. The composition of claim 95, wherein the gRNA is a gNRA selected from the group consisting of 5′-GTAATGAGTTCTTCCAACTG-3′ (SEQ ID NO: 1), 5′-TTGGAAGAACTCA-3′ (SEQ ID NO: 2), 5′-GAGGCGTCCCCAGGT-3′ (SEQ ID NO: 3), or a prime editing (pe) gRNA (pegRNA).

98. The composition of claim 97, wherein the prime editor comprises a CRISPR/Cas nuclease linked to a reverse transcriptase.

99. The composition of claim 98, wherein the CRISPR/Cas nuclease is catalytically impaired.

100. The composition of claim 98 or 99, wherein the CRISPR/Cas nuclease is a Cas9 nuclease.

101. The composition of claim 100, wherein the Cas9 nuclease is isolated or derived from Streptococcus pyogenes (spCas9).

102. The composition of claim 95, further comprising a second-strand nicking sgRNA.

103. A nucleic acid comprising:
a sequence encoding a first gRNA of claim 89,
a sequence encoding a prime editor,
a sequence encoding a first promoter, wherein the first promoter drives expression of the sequence encoding the first gRNA, and
a sequence encoding a second promoter, wherein the second promoter drives expression of the sequence encoding the prime editor.

104. The nucleic acid of claim 103, wherein the prime editor comprises a CRISPR/Cas nuclease linked to a reverse transcriptase.

105. The nucleic acid of claim 104, wherein the CRISPR/Cas nuclease is catalytically impaired.

106. The nucleic acid of claim 104, wherein the CRISPR/Cas nuclease is a Cas9 nuclease.

107. The nucleic acid of claim 106, wherein the Cas9 nuclease is isolated or derived from Streptococcus pyogenes (spCas9).

108. The nucleic acid of claim 103, further comprising a sequence encoding a second-strand nicking sgRNA.

109. The nucleic acid of claim 103, wherein at least one of the sequence encoding the first promoter and the sequence encoding the second promoter comprises a cell-type specific promoter.

110. The nucleic acid of claim 109, wherein the cell-type specific promoter is a muscle-specific promoter.

111. The nucleic acid of claim 110, wherein the muscle-specific promoter is a CK8 promoter.

112. The nucleic acid of claim 110, wherein the muscle-specific promoter is a CK8e promoter.

113. The nucleic acid of claim 103, wherein the sequence encoding the first promoter comprises a sequence encoding a U6 promoter, an H1 promoter, or a 7SK promoter.

114. The nucleic acid of claim 103, wherein the nucleic acid comprises a DNA sequence.

115. The nucleic acid of claim 103, wherein the nucleic acid comprises an RNA sequence.

116. The nucleic acid of claim 103, wherein the nucleic acid further comprises a polyadenosine (polyA) sequence.

117. The nucleic acid of claim 116, wherein the polyA sequence is a mini polyA sequence.

118. A cell comprising the nucleic acid of claim 103.

119. A composition comprising the nucleic acid of claim 103.

120. A cell comprising the composition of claim 119.

121. A composition comprising the cell of claim 120.

122. A vector comprising the nucleic acid of claim 103.

123. The vector of claim 122, wherein the vector further comprises a sequence encoding an inverted terminal repeat (ITR) of a transposable element.

124. The vector of claim 123, wherein the transposable element is a transposon.

125. The vector of claim 124, wherein the transposon is a Tn7 transposon.

126. The vector of claim 125, wherein the vector further comprises a sequence encoding a 5′ ITR of a T7 transposon and a sequence encoding a 3′ ITR of a T7 transposon.

127. The vector of claim 122, wherein the vector is a non-viral vector.

128. The vector of claim 127, wherein the non-viral vector is a plasmid.

129. The vector of claim 122, wherein the vector is a viral vector.

130. The vector of claim 129, wherein the viral vector is an adeno-associated viral (AAV) vector.

131. The vector of claim 130, wherein the AAV vector is replication-defective or conditionally replication defective.

132. The vector of claim 130, wherein the AAV vector is a recombinant AAV vector.

133. The vector of claim 130, wherein the AAV vector comprises a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11) or any combination thereof.

134. The vector of claim 130, wherein the AAV vector comprises a sequence isolated or derived from an AAV vector of serotype 9 (AAV9).

135. The vector of claim 130, wherein the AAV vector comprises a sequence isolated or derived from an AAV vector of serotype 2 (AAV2).

136. The vector of claim 130, wherein the AAV vector comprises a sequence isolated or derived from an AAV2 and a sequence isolated or derived from an AAV9.

137. The vector of claim 122, wherein the vector is optimized for expression in mammalian cells.

138. The vector of claim 122, wherein the vector is optimized for expression in human cells.

139. A composition comprising the vector of claim 122.

140. The composition of claim 139, further comprising a pharmaceutically acceptable carrier.

141. A cell comprising the composition of claim 46.

142. The cell of claim 141, wherein the cell is a human cell.

143. The cell of claim 141, wherein the cell is a muscle cell or satellite cell.

144. The cell of claim 141, wherein the cell is an induced pluripotent stem (iPS) cell.

145. A composition comprising the cell of claim 141.

146. A method for correcting a dystrophin defect, the method comprising contacting a cell with a composition of claim 139 under conditions suitable for expression of the first gRNA and the prime editor, wherein the first gRNA forms a complex with the prime editor, wherein the complex modifies a dystrophin splice site thereby inducing selective skipping of a DMD exon.

147. A cell produced by the method of claim 146.

148. A method of treating muscular dystrophy in a subject in need thereof, the method comprising administering to the subject a therapeutically effective amount of a composition of claim 139.

149. The method of claim 148, wherein the composition is administered locally.

150. The method of claim 148, wherein the composition is administered directly to a muscle tissue.

151. The method of claim 148, wherein the composition is administered by an intramuscular infusion or injection.

152. The method of claim 148, wherein the composition is administered systemically.

153. The method of claim 152, wherein the composition is administered by an intravenous infusion or injection.

154. The method of claim 148, wherein, following administration of the composition, the subject exhibits normal dystrophin-positive myofibers, and mosaic dystrophin-positive myofibers containing centralized nuclei, or a combination thereof.

155. The method of claim 148, wherein, following administration of the composition, the subject exhibits an emergence or an increase in a level of abundance of normal dystrophin-positive myofibers when compared to an absence or an level of abundance of normal dystrophin-positive myofibers prior to administration of the composition.

156. The method of claim 148, wherein, following administration of the composition, the subject exhibits an emergence or an increase in a level of abundance of mosaic dystrophin-positive myofibers containing centralized nuclei when compared to an absence or an level of abundance of mosaic dystrophin-positive myofibers containing centralized nuclei prior to administration of the composition.

157. The method of claim 148, wherein, following administration of the composition, the subject exhibits a decreased serum CK level when compared to a serum CK level prior to administration of the composition.

158. The method of claim 146, wherein, following administration of the composition, the subject exhibits improved grip strength when compared to a grip strength prior to administration of the composition.

159. The method of claim 146, wherein the subject is a neonate, an infant, a child, a young adult, or an adult.

160. The method of claim 146, wherein the subject has muscular dystrophy.

161. The method of claim 146, wherein the subject is a genetic carrier for muscular dystrophy.

162. The method of claim 146, wherein the subject is male.

163. The method of claim 146, wherein the subject is female.

164. The method of claim 146, wherein the subject appears to be asymptomatic and wherein a genetic diagnosis reveals a mutation in one or both copies of a DMD gene that impairs function of the DMD gene product.

165. The method of claim 146, wherein the subject presents an early sign or symptom of muscular dystrophy.

166. The method of claim 165, wherein the early sign or symptom of muscular dystrophy comprises loss of muscle mass or proximal muscle weakness.

167. The method of claim 166, wherein the loss of muscle mass or proximal muscle weakness occurs in one or both leg(s) and/or a pelvis, followed by one or more upper body muscle(s).

168. The method of claim 167, wherein the early sign or symptom of muscular dystrophy further comprises pseudohypertrophy, low endurance, difficulty standing, difficulty walking, difficulty ascending a staircase or a combination thereof.

169. The method of claim 146, wherein the subject presents a progressive sign or symptom of muscular dystrophy.

170. The method of claim 169, wherein the progressive sign or symptom of muscular dystrophy comprises muscle tissue wasting, replacement of muscle tissue with fat, or replacement of muscle tissue with fibrotic tissue.

171. The method of claim 146, wherein the subject presents a later sign or symptom of muscular dystrophy.

172. The method of claim 171, wherein the later sign or symptom of muscular dystrophy comprises abnormal bone development, curvature of the spine, loss of movement, and paralysis.

173. The method of claim 146, wherein the subject presents a neurological sign or symptom of muscular dystrophy.

174. The method of claim 173, wherein the neurological sign or symptom of muscular dystrophy comprises intellectual impairment and paralysis.

175. The method of claim 146, wherein the administration of the composition occurs prior to the subject presenting one or more progressive, later or neurological signs or symptoms of muscular dystrophy.

176. The method of claim 146, wherein the subject is less than 10 years old.

177. The method of claim 176, wherein the subject is less than 5 years old.

178. The method of claim 177, wherein the subject is less than 2 years old.

179. (canceled)


SEQUENCE LISTING (243 sequences)

SEQ ID NO: 1 - DNA, 20 bp, Artificial sequence
  Synthetic oligo
gtaatgagttcttccaactg

SEQ ID NO: 2 - DNA, 13 bp, Artificial sequence
  Synthetic oligo
ttggaagaactca

SEQ ID NO: 3 - DNA, 15 bp, Artificial sequence
  Synthetic oligo
gaggcgtccccaggt

SEQ ID NO: 4 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 5 - protein, 11 aa, Artificial sequence
  Synthetic peptide
MLWWEEVEDCY

SEQ ID NO: 6 - protein, 7 aa, Artificial sequence
  Synthetic peptide
MSEVSSD

SEQ ID NO: 7 - protein, 16 aa, Artificial sequence
  Synthetic peptide
MTEIILLIFFPAYFLN

SEQ ID NO: 8 - protein, 13 aa, Artificial sequence
  Synthetic peptide
MSARKLRNLSYKK

SEQ ID NO: 9 - protein, 10 aa, Artificial sequence
  Synthetic peptide
MLHRKTYHVK

SEQ ID NO: 10 - protein, 2158 aa, Mus musculus
MWWVDCYRDVKKTTKWNASKGKHDNSDDGKRDGTGKKKGSTRVHANNVNKARVKNNVDVNGSTDVDGNHKTGWNHWVKNVMKTMAGTNSKSWVRSTRNYVNVNTSSWSDGANAHSHRDDWNSVVSHSATRHANAKCGKDDVATTYDKKSMYTSVVSAVMRTSSKVTRHHHMHYSTVSAGYTSSSKRKSYATAAYVATSDSTSYSHARDKSDSSMTVNDSYTAVSWSADTRAGSNDVVKHAHGMMDTSHGVGNVGSVGKGKSDAVMNNSRWCRVASMKSKHKVMDNKKDDWTKTRTKKMGDDKCVHKVDVRVNSTHMVVVVDSSGDHATAAKVGDRWANCRWTDRWVDKWHTCSTWSKDAMKNTSGKDNMMSSHKSTKDKKKTMKSSNDSAKNKSVTKMWMNARWDNTKKSSASAVTTTSTTTVMTVTMVTTRMVKHAKKRTVDSRKRDVDTHSWTRSAVSSAVYRKGNSDKVNAARKAKRKDASRSAAVMANGVNASRASNSRWTCSRVNWYTNTYNMTTTANKTSTTSTAKSKCKDVNRSAKSKKGGMDADVATNHNHDGVRAKKTDTMRYTMSSRTWSSKSVYSVTYMRGKASSKNGNYSDTVKMAKKASCKYSGHWKKSSVSCKHMNKRKNHKTKWMAVDVKWAGDAKKKCRVGDTSNSVNGGKKSAASRTRNTWDHCRVYTRKAKAGDKTVSKDSMHWMTAYRDYKTDTAVMKRAKAKTKVKTTVNSVAHASAAKKTTTNYWCTRNGKCKTVWACWHSYKANKWNVKKTMNVAGTVSNMHHSNNRATTDGGVMDNTNSRWRHAVRKKSSAKSHSDKAAYTDKVDAAMAKSDTSHSMKKHNGKDANRVSDVAKKDVSMKRKANRSKMDVKMHATKSVVSSHCVNYKSSVKSVMVKTGRVKKTNKDRVTAKHYNGAKVTRKKCKSRKMRKMNVTWAATDTTKRSAVGMSNDSVAWGKATKKKAHKSVTGSKMVGKKTVDKSNSNWAVTSRVWNYKHMTDNTKWHADDSKKKKDKRKAMNDMRKVDSTRDAAKMANRGDHCRKVVSNRRAASHRKTGKASKNSDKAGVNKDNKDMSDNGTVNRGDNRTDRKRKKTKHNAKDRSRRKKASHWYYKRADDKCDKKASRDRKKDRKKKNAVRRAGSNGAAMAVTSKRWRSNARRNAHTHTMVVTTDMDVSYVSTYTSHASVDHNTCAKDDKSKNKDNSGRDHKKKTAASATSMKVKVAVAMDGKHRMYKRGRDRSVKWRHHYDMKVNWNVKKTNNWHAKYKWYKDGGRAVVRTNATGSSKTDVNKGSSRWHDCKARRKRKNVSRDNVWADNATGDKVKARGKNTGGAVVSARDKKKKTNWKVSRAKGVHKDRDHWSRNYNSAGDKVTVHGKADVRSKGHYKKSTVKRKDRSWAVNHRRTKDRAGSTTGASASTVTVTSVVTKTVSKMSSVAADNRAWTTDWSDRVKSRVMVGDDNMKKATDRRTAANKNKTSNARTTDRRWDVNRRNMKDSTWAKAVGVRGKDSWKGHTVDAKKTTKAKDRRSVDVANDAKRDYSADDTRKVHMTNNTSWGNHKRVSAATHRDKSWTATTANVDASRKKDSRGVRMKWDGTHTDYHNDNGKRSGSDARRDNMNKWSKKSNRSHASSDWKRHSVWKDDSRAGGDAVKNDHRAKRKTKVMSTTVRTGKYRRANVTRRKAVNAWDKNRSADWRKDARAADDKRAVKGSWVGDDSDHKVKARGAKNVNRVNDAHTTGSYNSTDNTRWRVAVDRVRHAHRDGASHSTSVGWRASNKVYYNHTTTCWDHKMTYSADNNVRSAYRTAMKRRKACDSSAACDADHNKNDMDNCTTYDRHNNVNVCVDMCNWNVYDTGRTGRRVSKTGSCKAHDKYRYKVASSTGCDRRGHDSRGVASGGSNSVRSCANNKAADWMRSMVWVHRVAAATAKHAKCNCKCGRYRSKHNYDCSCSGRVAKGHKMHYMVYCTTTSGDVRDAKVKNKRTKRYAKHRMGYVTVGDNMTVTNWVDSAASSSHDDTHSRHYASRAMNSNGSYNDSSNSDDHHYCSNDSSRSASSRGRADNRNAYDRKHHKGSSMMTSSRDAAAKRHKGRARMDHNKSHRRAAKVNGTTVSSSTSRSDSSMRVVGSTSSMGDSDTSTGVMNNSSSRGRNAGKMRDTM

SEQ ID NO: 11 - DNA, 20 bp, Artificial sequence
  Synthetic nucleic acid
atacttacaggctccaatag

SEQ ID NO: 12 - DNA, 20 bp, Artificial sequence
  Synthetic nucleic acid
gttcttacaggcaacaatgc

SEQ ID NO: 13 - DNA, 20 bp, Artificial sequence
  Synthetic nucleic acid
acaggcaacaatgcaggatt

SEQ ID NO: 14 - DNA, 20 bp, Artificial sequence
  Synthetic nucleic acid
aaatattttagctcctactc

SEQ ID NO: 15 - DNA, 20 bp, Artificial sequence
  Synthetic nucleic acid
attttagctcctactcagac

SEQ ID NO: 16 - DNA, 18 bp, Artificial sequence
  Synthetic nucleic acid
tttagctcctactcagac

SEQ ID NO: 17 - DNA, 20 bp, Artificial sequence
  Synthetic nucleic acid
ttctcataccttctgcttga

SEQ ID NO: 18 - DNA, 18 bp, Artificial sequence
  Synthetic nucleic acid
tttctcataccttctgct

SEQ ID NO: 19 - DNA, 20 bp, Artificial sequence
  Synthetic nucleic acid
tggtatcttacaggaactcc

SEQ ID NO: 20 - DNA, 20 bp, Artificial sequence
  Synthetic nucleic acid
ggtatcttacaggaactcca

SEQ ID NO: 21 - DNA, 20 bp, Artificial sequence
  Synthetic nucleic acid
atcttacaggaactccagga

SEQ ID NO: 22 - DNA, 20 bp, Artificial sequence
  Synthetic nucleic acid
tcttacaggaactccaggat

SEQ ID NO: 23 - DNA, 20 bp, Artificial sequence
  Synthetic nucleic acid
caggaactccaggatggcat

SEQ ID NO: 24 - DNA, 20 bp, Artificial sequence
  Synthetic nucleic acid
aggaactccaggatggcatt

SEQ ID NO: 25 - DNA, 20 bp, Artificial sequence
  Synthetic nucleic acid
acctcttttttctgtctgac

SEQ ID NO: 26 - DNA, 20 bp, Artificial sequence
  Synthetic nucleic acid
ccctacctcttttttctgtc

SEQ ID NO: 27 - DNA, 20 bp, Artificial sequence
  Synthetic nucleic acid
gtcgccctacctcttttttc

SEQ ID NO: 28 - DNA, 20 bp, Artificial sequence
  Synthetic nucleic acid
tttatattacagaatataaa

SEQ ID NO: 29 - DNA, 20 bp, Artificial sequence
  Synthetic nucleic acid
tattacagaatataaaagat

SEQ ID NO: 30 - DNA, 20 bp, Artificial sequence
  Synthetic nucleic acid
tacctacccttgtcggtcct

SEQ ID NO: 31 - DNA, 20 bp, Artificial sequence
  Synthetic nucleic acid
atgtgttacctacccttgtc

SEQ ID NO: 32 - DNA, 20 bp, Artificial sequence
  Synthetic nucleic acid
tatgtgttacctacccttgt

SEQ ID NO: 33 - DNA, 20 bp, Artificial sequence
  Synthetic nucleic acid
atatatgtgttacctaccct

SEQ ID NO: 34 - DNA, 20 bp, Artificial sequence
  Synthetic nucleic acid
gcttttacctgcaggcgatt

SEQ ID NO: 35 - DNA, 20 bp, Artificial sequence
  Synthetic nucleic acid
ttacctgcaggcgatttgac

SEQ ID NO: 36 - DNA, 20 bp, Artificial sequence
  Synthetic nucleic acid
tgcaggcgatttgacagatc

SEQ ID NO: 37 - DNA, 20 bp, Artificial sequence
  Synthetic nucleic acid
aggcgatttgacagatctgt

SEQ ID NO: 38 - DNA, 20 bp, Artificial sequence
  Synthetic nucleic acid
acttaccttaagataccatt

SEQ ID NO: 39 - DNA, 20 bp, Artificial sequence
  Synthetic nucleic acid
caaatcaaagacttacctta

SEQ ID NO: 40 - DNA, 20 bp, Artificial sequence
  Synthetic nucleic acid
tattcttctttctccaggct

SEQ ID NO: 41 - DNA, 20 bp, Artificial sequence
  Synthetic nucleic acid
tcttctttctccaggctaga

SEQ ID NO: 42 - DNA, 20 bp, Artificial sequence
  Synthetic nucleic acid
ctccaggctagaagaacaaa

SEQ ID NO: 43 - DNA, 20 bp, Artificial sequence
  Synthetic nucleic acid
aaattaccttgacttgctca

SEQ ID NO: 44 - DNA, 20 bp, Artificial sequence
  Synthetic nucleic acid
aaaataaaattaccttgact

SEQ ID NO: 45 - DNA, 20 bp, Artificial sequence
  Synthetic nucleic acid
ttgagaaaataaaattacct

SEQ ID NO: 46 - DNA, 20 bp, Artificial sequence
  Synthetic nucleic acid
tgcttttctgttaaagagga

SEQ ID NO: 47 - DNA, 20 bp, Artificial sequence
  Synthetic nucleic acid
tttctgttaaagaggaagtt

SEQ ID NO: 48 - DNA, 20 bp, Artificial sequence
  Synthetic nucleic acid
ctgttaaagaggaagttaga

SEQ ID NO: 49 - DNA, 20 bp, Artificial sequence
  Synthetic nucleic acid
aaagaggaagttagaagatc

SEQ ID NO: 50 - DNA, 20 bp, Artificial sequence
  Synthetic nucleic acid
agaggaagttagaagatctg

SEQ ID NO: 51 - DNA, 20 bp, Artificial sequence
  Synthetic nucleic acid
tacaggctccaatagtggtc

SEQ ID NO: 52 - DNA, 20 bp, Artificial sequence
  Synthetic nucleic acid
tacttacaggctccaatagt

SEQ ID NO: 53 - DNA, 20 bp, Artificial sequence
  Synthetic nucleic acid
gtatacttacaggctccaat

SEQ ID NO: 54 - DNA, 20 bp, Artificial sequence
  Synthetic nucleic acid
tttgttcttacaggcaacaa

SEQ ID NO: 55 - DNA, 20 bp, Artificial sequence
  Synthetic nucleic acid
ttcttacaggcaacaatgca

SEQ ID NO: 56 - DNA, 20 bp, Artificial sequence
  Synthetic nucleic acid
caggcaacaatgcaggattt

SEQ ID NO: 57 - DNA, 20 bp, Artificial sequence
  Synthetic nucleic acid
ttacttcgatccgtaatgat

SEQ ID NO: 58 - DNA, 20 bp, Artificial sequence
  Synthetic nucleic acid
aaacttacttcgatccgtaa

SEQ ID NO: 59 - DNA, 20 bp, Artificial sequence
  Synthetic nucleic acid
ttaaaaaacttacttcgatc

SEQ ID NO: 60 - DNA, 20 bp, Artificial sequence
  Synthetic nucleic acid
tattctagttgaaagaattc

SEQ ID NO: 61 - DNA, 20 bp, Artificial sequence
  Synthetic nucleic acid
agttgaaagaattcagaatc

SEQ ID NO: 62 - DNA, 20 bp, Artificial sequence
  Synthetic nucleic acid
gatactaaccttggtttctg

SEQ ID NO: 63 - DNA, 20 bp, Artificial sequence
  Synthetic nucleic acid
ttgatactaaccttggtttc

SEQ ID NO: 64 - DNA, 21 bp, Artificial sequence
  Synthetic nucleic acid
agctcctactcagactgttac

SEQ ID NO: 65 - DNA, 22 bp, Artificial sequence
  Synthetic nucleic acid
agctcctactcagactgttact

SEQ ID NO: 66 - DNA, 19 bp, Artificial sequence
  Synthetic nucleic acid
tttctcataccttctgctt

SEQ ID NO: 67 - DNA, 21 bp, Artificial sequence
  Synthetic nucleic acid
tttggtatcttacaggaactc

SEQ ID NO: 68 - DNA, 21 bp, Artificial sequence
  Synthetic nucleic acid
gtatcttacaggaactccagg

SEQ ID NO: 69 - DNA, 21 bp, Artificial sequence
  Synthetic nucleic acid
tacaggaactccaggatggca

SEQ ID NO: 70 - DNA, 21 bp, Artificial sequence
  Synthetic nucleic acid
acaggaactccaggatggcat

SEQ ID NO: 71 - DNA, 21 bp, Artificial sequence
  Synthetic nucleic acid
tttattctagttgaaagaatt

SEQ ID NO: 72 - DNA, 21 bp, Artificial sequence
  Synthetic nucleic acid
agttgaaagaattcagaatca

SEQ ID NO: 73 - DNA, 22 bp, Artificial sequence
  Synthetic nucleic acid
agttgaaagaattcagaatcag

SEQ ID NO: 74 - DNA, 21 bp, Artificial sequence
  Synthetic nucleic acid
tttgatactaaccttggtttc

SEQ ID NO: 75 - DNA, 21 bp, Artificial sequence
  Synthetic nucleic acid
gcaggcgatttgacagatctg

SEQ ID NO: 76 - DNA, 19 bp, Artificial sequence
  Synthetic nucleic acid
tttacctgcaggcgatttg

SEQ ID NO: 77 - DNA, 22 bp, Artificial sequence
  Synthetic nucleic acid
aggcgatttgacagatctgttg

SEQ ID NO: 78 - DNA, 21 bp, Artificial sequence
  Synthetic nucleic acid
ttctccaggctagaagaacaa

SEQ ID NO: 79 - DNA, 21 bp, Artificial sequence
  Synthetic nucleic acid
ttgttcttacaggcaacaatg

SEQ ID NO: 80 - DNA, 21 bp, Artificial sequence
  Synthetic nucleic acid
ttacaggcaacaatgcaggat

SEQ ID NO: 81 - DNA, 21 bp, Artificial sequence
  Synthetic nucleic acid
tacaggcaacaatgcaggatt

SEQ ID NO: 82 - DNA, 21 bp, Artificial sequence
  Synthetic nucleic acid
aaaaaacttacttcgatccgt

SEQ ID NO: 83 - DNA, 21 bp, Artificial sequence
  Synthetic nucleic acid
gttaaaaaacttacttcgatc

SEQ ID NO: 84 - DNA, 21 bp, Artificial sequence
  Synthetic nucleic acid
ttaaagaggaagttagaagat

SEQ ID NO: 85 - DNA, 21 bp, Artificial sequence
  Synthetic nucleic acid
tttctgttaaagaggaagtta

SEQ ID NO: 86 - DNA, 21 bp, Artificial sequence
  Synthetic nucleic acid
tacttacaggctccaatagtg

SEQ ID NO: 87 - DNA, 21 bp, Artificial sequence
  Synthetic nucleic acid
gtatacttacaggctccaata

SEQ ID NO: 88 - DNA, 21 bp, Artificial sequence
  Synthetic nucleic acid
agtatacttacaggctccaat

SEQ ID NO: 89 - DNA, 21 bp, Artificial sequence
  Synthetic nucleic acid
tccagtatacttacaggctcc

SEQ ID NO: 90 - DNA, 21 bp, Artificial sequence
  Synthetic nucleic acid
tttatattacagaatataaaa

SEQ ID NO: 91 - DNA, 24 bp, Artificial sequence
  Synthetic nucleic acid
cgtccccaggtttggaagaactca

SEQ ID NO: 92 - DNA, 25 bp, Artificial sequence
  Synthetic nucleic acid
gcgtccccaggtttggaagaactca

SEQ ID NO: 93 - DNA, 26 bp, Artificial sequence
  Synthetic nucleic acid
ggcgtccccaggtttggaagaactca

SEQ ID NO: 94 - DNA, 27 bp, Artificial sequence
  Synthetic nucleic acid
aggcgtccccaggtttggaagaactca

SEQ ID NO: 95 - DNA, 28 bp, Artificial sequence
  Synthetic nucleic acid
gaggcgtccccaggtttggaagaactca

SEQ ID NO: 96 - DNA, 29 bp, Artificial sequence
  Synthetic nucleic acid
agaggcgtccccaggtttggaagaactca

SEQ ID NO: 97 - DNA, 26 bp, Artificial sequence
  Synthetic nucleic acid
gaggcgtccccaggtttggaagaact

SEQ ID NO: 98 - DNA, 27 bp, Artificial sequence
  Synthetic nucleic acid
gaggcgtccccaggtttggaagaactc

SEQ ID NO: 99 - DNA, 29 bp, Artificial sequence
  Synthetic nucleic acid
gaggcgtccccaggtttggaagaactcat

SEQ ID NO: 100 - DNA, 30 bp, Artificial sequence
  Synthetic nucleic acid
gaggcgtccccaggtttggaagaactcatt

SEQ ID NO: 101 - DNA, 20 bp, Artificial sequence
  Synthetic nucleic acid
gttcttacaggcaacaatgc

SEQ ID NO: 102 - DNA, 20 bp, Artificial sequence
  Synthetic nucleic acid
aaacaagaccagcaatcaag

SEQ ID NO: 103 - DNA, 450 bp, Artificial sequence
  Synthetic nucleic acid
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 104 - DNA, 436 bp, Artificial sequence
  Synthetic nucleic acid
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 105 - protein, 18 aa, Thosea asigna
EGRGSLLTCGDVEENPGP

SEQ ID NO: 106 - protein, 20 aa, Artificial sequence
  Synthetic amino acid
QCTNYALLKLAGDVESNPGP

SEQ ID NO: 107 - protein, 19 aa, Artificial sequence
  Synthetic amino acid
ATNFSLLKQAGDVEENPGP

SEQ ID NO: 108 - protein, 23 aa, Artificial sequence
  Synthetic amino acid
PVKQLLNFDLLKLAGDVESNPGP

SEQ ID NO: 109 - protein, 41 aa, Artificial sequence
  Synthetic amino acid
RMRKFKNKGKDTAELRRRRVEVSVELRKAKKDEQILKRRNV

SEQ ID NO: 110 - protein, 8 aa, Artificial sequence
  Synthetic amino acid
VSRKRPRP

SEQ ID NO: 111 - protein, 8 aa, Artificial sequence
  Synthetic amino acid
PPKKARED

SEQ ID NO: 112 - protein, 8 aa, Artificial sequence
  Synthetic amino acid
PQPKKKPL

SEQ ID NO: 113 - protein, 12 aa, Artificial sequence
  Synthetic amino acid
SALIKKKKKMAP

SEQ ID NO: 114 - protein, 5 aa, Artificial sequence
  Synthetic amino acid
DRLRR

SEQ ID NO: 115 - protein, 7 aa, Artificial sequence
  Synthetic amino acid
PKQKKRK

SEQ ID NO: 116 - protein, 10 aa, Artificial sequence
  Synthetic amino acid
RKLKKKIKKL

SEQ ID NO: 117 - protein, 10 aa, Artificial sequence
  Synthetic amino acid
REKKKFLKRR

SEQ ID NO: 118 - protein, 20 aa, Artificial sequence
  Synthetic amino acid
KRKGDEVDGVDEVAKKKSKK

SEQ ID NO: 119 - protein, 17 aa, Artificial sequence
  Synthetic amino acid
RKCLQAGMNLEARKTKK

SEQ ID NO: 120 - DNA, 4101 bp, Artificial sequence
  Synthetic nucleic acid
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 121 - DNA, 115 bp, Artificial sequence
  Synthetic nucleic acid
ggccactccctctctgcgcgctcgctcgctcactgaggccgggcgaccaaaggtcgcccgacgcccgggctttgcccgggcggcctcagtgagcgagcgagcgcgcagagaggga

SEQ ID NO: 122 - DNA, 141 bp, Artificial sequence
  Synthetic nucleic acid
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 123 - DNA, 141 bp, Artificial sequence
  Synthetic nucleic acid
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 124 - DNA, 60 bp, Artificial sequence
  Synthetic nucleic acid
tagcaataaaggatcgtttattttcattggaagcgtgtgttggttttttgatcaggcgcg

SEQ ID NO: 125 - DNA, 48 bp, Artificial sequence
  Synthetic nucleic acid
aagcgtcctgctgctactaagaaagctggtcaagctaagaaaaagaaa

SEQ ID NO: 126 - DNA, 51 bp, Artificial sequence
  Synthetic nucleic acid
atggccccaaagaagaagcggaaggtcggtatccacggagtcccagcagcc

SEQ ID NO: 127 - DNA, 166 bp, Artificial sequence
  Synthetic nucleic acid
tgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggtt

SEQ ID NO: 128 - DNA, 225 bp, Artificial sequence
  Synthetic nucleic acid
gaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccaca

SEQ ID NO: 129 - DNA, 1155 bp, Artificial sequence
  Synthetic nucleic acid
catgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccg

SEQ ID NO: 130 - DNA, 20 bp, Artificial sequence
  Synthetic nucleic acid
ctattaaagaggaagttaga

SEQ ID NO: 131 - DNA, 20 bp, Artificial sequence
  Synthetic nucleic acid
aaagaggaagttagaagatc

SEQ ID NO: 132 - DNA, 20 bp, Artificial sequence
  Synthetic nucleic acid
tacaggctccagtagtgctc

SEQ ID NO: 133 - DNA, 20 bp, Artificial sequence
  Synthetic nucleic acid
gtacttacaggctccagtag

SEQ ID NO: 134 - DNA, 20 bp, Artificial sequence
  Synthetic nucleic acid
atgtacttacaggctccagt

SEQ ID NO: 135 - DNA, 20 bp, Artificial sequence
  Synthetic nucleic acid
ttctttcaggcaacactgca

SEQ ID NO: 136 - DNA, 20 bp, Artificial sequence
  Synthetic nucleic acid
tcaggcaacactgcaagatt

SEQ ID NO: 137 - DNA, 20 bp, Artificial sequence
  Synthetic nucleic acid
ttacttcgatcagtaatgat

SEQ ID NO: 138 - DNA, 20 bp, Artificial sequence
  Synthetic nucleic acid
agacttacttcgatcagtaa

SEQ ID NO: 139 - DNA, 20 bp, Artificial sequence
  Synthetic nucleic acid
atacttacaggctccaatag

SEQ ID NO: 140 - DNA, 20 bp, Artificial sequence
  Synthetic nucleic acid
gttcttacaggcaacaatgc

SEQ ID NO: 141 - DNA, 20 bp, Artificial sequence
  Synthetic nucleic acid
acaggcaacaatgcaggatt

SEQ ID NO: 142 - DNA, 20 bp, Artificial sequence
  Synthetic nucleic acid
attttagctcctactcagac

SEQ ID NO: 143 - DNA, 20 bp, Artificial sequence
  Synthetic nucleic acid
ttctcataccttctgcttga

SEQ ID NO: 144 - DNA, 20 bp, Artificial sequence
  Synthetic nucleic acid
atcttacaggaactccagga

SEQ ID NO: 145 - DNA, 20 bp, Artificial sequence
  Synthetic nucleic acid
tcttacaggaactccaggat

SEQ ID NO: 146 - DNA, 20 bp, Artificial sequence
  Synthetic nucleic acid
caggaactccaggatggcat

SEQ ID NO: 147 - DNA, 20 bp, Artificial sequence
  Synthetic nucleic acid
aggaactccaggatggcatt

SEQ ID NO: 148 - DNA, 20 bp, Artificial sequence
  Synthetic nucleic acid
ccctacctcttttttctgtc

SEQ ID NO: 149 - DNA, 20 bp, Artificial sequence
  Synthetic nucleic acid
ctgaaatgatggccaaggat

SEQ ID NO: 150 - DNA, 20 bp, Artificial sequence
  Synthetic nucleic acid
ccacaaagccatccagtttt

SEQ ID NO: 151 - DNA, 26 bp, Artificial sequence
  Synthetic nucleic acid
aactggagattcatattgttatcctg

SEQ ID NO: 152 - DNA, 25 bp, Artificial sequence
  Synthetic nucleic acid
gccctaccttcatgaagattacaac

SEQ ID NO: 153 - DNA, 20 bp, Artificial sequence
  Synthetic nucleic acid
atgcctggagaaagggtttt

SEQ ID NO: 154 - DNA, 20 bp, Artificial sequence
  Synthetic nucleic acid
ctctcccacaatctcccttg

SEQ ID NO: 155 - DNA, 27 bp, Artificial sequence
  Synthetic nucleic acid
gaatgaaacatatttcctgttaaattg

SEQ ID NO: 156 - DNA, 19 bp, Artificial sequence
  Synthetic nucleic acid
gaaagatggcccaggaaga

SEQ ID NO: 157 - DNA, 26 bp, Artificial sequence
  Synthetic nucleic acid
aggttcacttaaaagattttaggcag

SEQ ID NO: 158 - DNA, 26 bp, Artificial sequence
  Synthetic nucleic acid
taacatttcattcaactgttgtctcc

SEQ ID NO: 159 - DNA, 20 bp, Artificial sequence
  Synthetic nucleic acid
taaaagaccttgggcagctt

SEQ ID NO: 160 - DNA, 20 bp, Artificial sequence
  Synthetic nucleic acid
gccaactgcttggtttctgt

SEQ ID NO: 161 - DNA, 20 bp, Artificial sequence
  Synthetic nucleic acid
cgtctgaatgcgcttgtctg

SEQ ID NO: 162 - DNA, 20 bp, Artificial sequence
  Synthetic nucleic acid
acagccgtccaatgtcaact

SEQ ID NO: 163 - DNA, 20 bp, Artificial sequence
  Synthetic nucleic acid
caagggttcctcgactgcat

SEQ ID NO: 164 - DNA, 20 bp, Artificial sequence
  Synthetic nucleic acid
tgtacatggaagctggccac

SEQ ID NO: 165 - DNA, 20 bp, Artificial sequence
  Synthetic nucleic acid
aggctgctggagacttagga

SEQ ID NO: 166 - DNA, 20 bp, Artificial sequence
  Synthetic nucleic acid
ctagagagccaggtggtcct

SEQ ID NO: 167 - DNA, 20 bp, Artificial sequence
  Synthetic nucleic acid
cctccagcaacttagggacc

SEQ ID NO: 168 - DNA, 20 bp, Artificial sequence
  Synthetic nucleic acid
ccttctccctgtctgccttg

SEQ ID NO: 169 - DNA, 20 bp, Artificial sequence
  Synthetic nucleic acid
ctgtggaaagcggagacact

SEQ ID NO: 170 - DNA, 20 bp, Artificial sequence
  Synthetic nucleic acid
ccaaccttctcagggcagtt

SEQ ID NO: 171 - DNA, 20 bp, Artificial sequence
  Synthetic nucleic acid
gaggacaactggaaggcctg

SEQ ID NO: 172 - DNA, 23 bp, Artificial sequence
  Synthetic nucleic acid
tctgtctgccagagttgttttct

SEQ ID NO: 173 - DNA, 20 bp, Artificial sequence
  Synthetic nucleic acid
tgcatcttaaccattaccat

SEQ ID NO: 174 - DNA, 20 bp, Artificial sequence
  Synthetic nucleic acid
gcacagacaacttagaagag

SEQ ID NO: 175 - DNA, 31 bp, Artificial sequence
  Synthetic nucleic acid
tgagcactactggagcctgtaagtacatttt

SEQ ID NO: 176 - DNA, 31 bp, Artificial sequence
  Synthetic nucleic acid
aaaatgtacttacaggctccagtagtgctca

SEQ ID NO: 177 - DNA, 31 bp, Artificial sequence
  Synthetic nucleic acid
  misc_feature (20)..(20): n is a, c, g, or t
tgagcactactggagcctgnaagtacatttt

SEQ ID NO: 178 - DNA, 33 bp, Artificial sequence
  Synthetic nucleic acid
ctttcctattaaagaggaagttagaagatctga

SEQ ID NO: 179 - DNA, 33 bp, Artificial sequence
  Synthetic nucleic acid
tcagatcttctaacttcctctttaataggaaag

SEQ ID NO: 180 - DNA, 33 bp, Artificial sequence
  Synthetic nucleic acid
actgagcactactggagcctgtaagtacatttt

SEQ ID NO: 181 - DNA, 33 bp, Artificial sequence
  Synthetic nucleic acid
aaaatgtacttacaggctccagtagtgctcagt

SEQ ID NO: 182 - DNA, 32 bp, Artificial sequence
  Synthetic nucleic acid
tttttttctttcaggcaacactgcaagattgg

SEQ ID NO: 183 - DNA, 32 bp, Artificial sequence
  Synthetic nucleic acid
ccaatcttgcagtgttgcctgaaagaaaaaaa

SEQ ID NO: 184 - DNA, 33 bp, Artificial sequence
  Synthetic nucleic acid
aacaatcattactgatcgaagtaagtctcttaa

SEQ ID NO: 185 - DNA, 33 bp, Artificial sequence
  Synthetic nucleic acid
ttaagagacttacttcgatcagtaatgattgtt

SEQ ID NO: 186 - DNA, 20 bp, Artificial sequence
  Synthetic nucleic acid
gtacctacagaatccagtag

SEQ ID NO: 187 - DNA, 20 bp, Artificial sequence
  Synthetic nucleic acid
acacttatagactccagtag

SEQ ID NO: 188 - DNA, 20 bp, Artificial sequence
  Synthetic nucleic acid
tttcttacaggctccagaaa

SEQ ID NO: 189 - DNA, 20 bp, Artificial sequence
  Synthetic nucleic acid
gtactcacaggatccagaag

SEQ ID NO: 190 - DNA, 20 bp, Artificial sequence
  Synthetic nucleic acid
gtgcttaaaagccccagtag

SEQ ID NO: 191 - DNA, 20 bp, Artificial sequence
  Synthetic nucleic acid
gtgcctacagagtccagtag

SEQ ID NO: 192 - DNA, 20 bp, Artificial sequence
  Synthetic nucleic acid
gtactcagagagtccagaag

SEQ ID NO: 193 - DNA, 20 bp, Artificial sequence
  Synthetic nucleic acid
atatttagaggctccagttg

SEQ ID NO: 194 - DNA, 30 bp, Artificial sequence
  Synthetic nucleic acid
actcagccagtgaaggcaacactgcaagat

SEQ ID NO: 195 - DNA, 30 bp, Artificial sequence
  Synthetic nucleic acid
tgaccactatggagcctgtaagtatactgg

SEQ ID NO: 196 - DNA, 31 bp, Artificial sequence
  Synthetic nucleic acid
ccagtatacttacaggctccaatagtggtca

SEQ ID NO: 197 - DNA, 31 bp, Artificial sequence
  Synthetic nucleic acid
tgaccactattggagcctgcaagtatactgg

SEQ ID NO: 198 - DNA, 32 bp, Artificial sequence
  Synthetic nucleic acid
tgttcttacaggcaacaatgcaggatttggaa

SEQ ID NO: 199 - DNA, 32 bp, Artificial sequence
  Synthetic nucleic acid
ttccaaatcctgcattgttgcctgtaagaaca

SEQ ID NO: 200 - DNA, 49 bp, Artificial sequence
  Synthetic nucleic acid
cgagatgatcatcaagcagaaggcaacaatgcaggatttggaacagagg

SEQ ID NO: 201 - DNA, 49 bp, Artificial sequence
  Synthetic nucleic acid
ggactgaccactattggagcctgcaacaatgcaggatttggaacagagg

SEQ ID NO: 202 - DNA, 49 bp, Artificial sequence
  Synthetic nucleic acid
accagccactcagccagtgaaggcaacaatgcaggatttggaacagagg

SEQ ID NO: 203 - DNA, 23 bp, Artificial sequence
  Synthetic nucleic acid
ccccagttggaagaactcattac

SEQ ID NO: 204 - DNA, 20 bp, Artificial sequence
  Synthetic nucleic acid
gtaatgagttcttccaactg

SEQ ID NO: 205 - DNA, 28 bp, Artificial sequence
  Synthetic nucleic acid
gaggcgtccccaggtttggaagaactca

SEQ ID NO: 206 - DNA, 17 bp, Artificial sequence
  Synthetic nucleic acid
gtaatgagttcttccaa

SEQ ID NO: 207 - DNA, 13 bp, Artificial sequence
  Synthetic nucleic acid
ctccgcaggggtc

SEQ ID NO: 208 - DNA, 26 bp, Artificial sequence
  Synthetic nucleic acid
gaggcgtccccagttggaagaactca

SEQ ID NO: 209 - DNA, 28 bp, Artificial sequence
  Synthetic nucleic acid
gaggcgtccccaggtttggaagaactca

SEQ ID NO: 210 - DNA, 20 bp, Artificial sequence
  Synthetic nucleic acid
gttcttacaggcaacaatgc

SEQ ID NO: 211 - DNA, 20 bp, Artificial sequence
  Synthetic nucleic acid
acaggcaacaatgcaggatt

SEQ ID NO: 212 - DNA, 20 bp, Artificial sequence
  Synthetic nucleic acid
caatgcaggatttggaacag

SEQ ID NO: 213 - DNA, 20 bp, Artificial sequence
  Synthetic nucleic acid
gtaatgagttcttccaactg

SEQ ID NO: 214 - DNA, 20 bp, Artificial sequence
  Synthetic nucleic acid
ggtaatgagttcttccaact

SEQ ID NO: 215 - DNA, 20 bp, Artificial sequence
  Synthetic nucleic acid
cggtaatgagttcttccaac

SEQ ID NO: 216 - DNA, 20 bp, Artificial sequence
  Synthetic nucleic acid
ggaacagaggcgtccccagt

SEQ ID NO: 217 - DNA, 20 bp, Artificial sequence
  Synthetic nucleic acid
gtttttcaaattttgggcag

SEQ ID NO: 218 - DNA, 20 bp, Artificial sequence
  Synthetic nucleic acid
ggtcttgtttttcaaatttt

SEQ ID NO: 219 - DNA, 20 bp, Artificial sequence
  Synthetic nucleic acid
tggtcttgtttttcaaattt

SEQ ID NO: 220 - DNA, 20 bp, Artificial sequence
  Synthetic nucleic acid
tgttctagcctcttgattgc

SEQ ID NO: 221 - DNA, 20 bp, Artificial sequence
  Synthetic nucleic acid
aaacaagaccagcaatcaag

SEQ ID NO: 222 - DNA, 20 bp, Artificial sequence
  Synthetic nucleic acid
agaggctagaacaatcatta

SEQ ID NO: 223 - DNA, 50 bp, Artificial sequence
  Synthetic nucleic acid
tttgcaaaaacccaaaatattttagctcctactcagactgttactctggt

SEQ ID NO: 224 - DNA, 50 bp, Artificial sequence
  Synthetic nucleic acid
accagagtaacagtctgagtaggagctaaaatattttgggtttttgcaaa

SEQ ID NO: 225 - DNA, 50 bp, Artificial sequence
  Synthetic nucleic acid
caacgagatgatcatcaagcagaaggtatgagaaaaaatgataaaagttg

SEQ ID NO: 226 - DNA, 50 bp, Artificial sequence
  Synthetic nucleic acid
caacttttatcattttttctcataccttctgcttgatgatcatctcgttg

SEQ ID NO: 227 - DNA, 69 bp, Artificial sequence
  Synthetic nucleic acid
cctggactgaccactattggagcctctcctactcagactgttactctggtgacacaacctgtggttact

SEQ ID NO: 228 - DNA, 43 bp, Artificial sequence
  Synthetic nucleic acid
ctcctactcaactgttactctggtgacacaacctgtggttact

SEQ ID NO: 229 - DNA, 33 bp, Artificial sequence
  Synthetic nucleic acid
actgttactgtggtgacacaacctgtggttact

SEQ ID NO: 230 - DNA, 50 bp, Artificial sequence
  Synthetic nucleic acid
gttttgcctttttggtatcttacaggaactccaggatggcattgggcagc

SEQ ID NO: 231 - DNA, 50 bp, Artificial sequence
  Synthetic nucleic acid
gctgcccaatgccatcctggagttcctgtaagataccaaaaaggcaaaac

SEQ ID NO: 232 - DNA, 50 bp, Artificial sequence
  Synthetic nucleic acid
aacagctgtcagacagaaaaaagaggtagggcgacagatctaataggaat

SEQ ID NO: 233 - DNA, 50 bp, Artificial sequence
  Synthetic nucleic acid
attcctattagatctgtcgccctacctcttttttctgtctgacagctgtt

SEQ ID NO: 234 - DNA, 40 bp, Artificial sequence
  Synthetic nucleic acid
atgtacaaggaccgacaagggctagaagaacaaaagaata

SEQ ID NO: 235 - DNA, 40 bp, Artificial sequence
  Synthetic nucleic acid
tttttggtatcttacaggaactccaggatggcattgggca

SEQ ID NO: 236 - DNA, 40 bp, Artificial sequence
  Synthetic nucleic acid
tgcccaatgccatcctggagttcctgtaagataccaaaaa

SEQ ID NO: 237 - DNA, 41 bp, Artificial sequence
  Synthetic nucleic acid
tttttggtattcttacaggaactccaggatggcattgggca

SEQ ID NO: 238 - DNA, 40 bp, Artificial sequence
  Synthetic nucleic acid
tttttggtatcttgcgggaactccaggatggcattgggca

SEQ ID NO: 239 - DNA, 40 bp, Artificial sequence; identical to SEQ ID NO: 238
  Synthetic nucleic acid

SEQ ID NO: 240 - DNA, 40 bp, Artificial sequence
  Synthetic nucleic acid
tttttggtatcttacaggggctccgggatggcattgggca

SEQ ID NO: 241 - DNA, 40 bp, Artificial sequence
  Synthetic nucleic acid
tttttggtatcttacaggaactccaggatcccattgggca

SEQ ID NO: 242 - DNA, 18 bp, Artificial sequence
  Synthetic nucleic acid
aaccattacttctaagtt

SEQ ID NO: 243 - DNA, 24 bp, Artificial sequence
  Synthetic nucleic acid
ctattggagcctgcaacaatgcag